(19) United States
(12) Patent Application Publication
(10) Pub. No.: US 2014/0377269 A1
(43) Pub. Date: Dec. 25, 2014

(54) MULTIVALENT ANTIBODY ANALOGS, AND METHODS OF THEIR PREPARATION AND USE

(71) Applicant: Adimab, LLC, Lebanon, NH (US)

(72) Inventors: Robert Mabry, Lebanon, NH (US); Paul Widboom, Lebanon, NH (US); Ross Connor, Lebanon, NH (US)

(73) Assignee: Adimab, LLC, Lebanon, NH (US)

(21) Appl. No.: 14/135,286

(22) Filed: Dec. 19, 2013

Related U.S. Application Data
(60) Provisional application No. 61/739,361, filed on Dec. 19, 2012.

(51) Int. Cl. C07K 16/46

(52) U.S. Cl. CPC C07K 16/468; C07K 2317/31
USPC 424/136.1; 530/387.3

(57) ABSTRACT

Multivalent antibody analogs that co-engage at least two different antigens or epitopes (also referred to “targets”, used interchangeably throughout), are provided, as well as methods for their production and use.

### CROSS-REFERENCE TO RELATED APPLICATION

This application claims the benefit of U.S. Provisional Application No. 61/739,361, filed Dec. 19, 2012; the entire contents of which are incorporated herein by reference.

### SEQUENCE LISTING

In accordance with 37 CFR §1.52(e)(5), the present specification makes reference to a Sequence Listing (submitted electronically as a .txt file named “2009186-0106_Sequence_Listing.txt” on Jan. 8, 2014). The .txt file was generated on Aug. 27, 2014 and is 147,295 bytes in size. The entire contents of the Sequence Listing are herein incorporated by reference.

### FIELD OF THE INVENTION

The present invention relates, inter alia, multivalent antibody analogs, methods of making and using the same.

### BACKGROUND OF THE INVENTION

All references cited herein, including patents, patent applications, and non-patent publications referenced throughout are hereby expressly incorporated by reference in their entirety for all purposes.

Antibodies and antibody-based molecules represent attractive candidates as diagnostic tools and therapeutics. To date more than 30 therapeutic monoclonal antibodies have been approved for and successfully applied in diverse indication areas including cancer, organ transplantation, autoimmune and inflammatory disorders, infectious disease, and cardiovascular disease.

However, the majority of these antibodies are monospecific antibodies, which recognize a single epitope and can be selected to either activate or repress the activity of a target molecule through this single epitope. Many physiological responses, however, require crosslinking, “cross-talk” or co-engagement of or between two or more different proteins or protein subunits to be triggered. An important example is the activation of heteromeric, cell-surface receptor complexes. For these receptor complexes, activation is normally achieved through ligand interaction with multiple domains on different proteins resulting in proximity-associated activation of one or both receptor components.

A desire to address and therapeutically exploit some of these more complex physiological processes, and disease states associated therewith, has stimulated significant effort towards generating multispecific antibodies that can co-engage multiple epitopes or antigens. One avenue that has received much attention is the engineering of additional and novel antigen binding sites into antibody-based drugs such that a single inventive multivalent antibody analog molecule co-engages two or more different antigen targets. Such non-native or alternate antibody formats that engage two or more different antigens are often referred to as multispecifics, such as bispecifics. Bispecific antibodies (BsAbs), with affinity towards two different epitopes on either the same or distinct antigens, have been previously described (reviewed by Holliger and Winter 1993 Curr. Opin. Biotech. 4, 446-449 (see also Poljak, R. J., et al. (1994) Structure 2:1121-1123; and Cao et al. (1998), Bioconjugate Chem. 9, 635-644)). Such antibodies may be particularly useful in, among other things, redirection of cytotoxic agents or immune effector cells to target sites, as tumors. A number of alternate antibody formats have been explored for bispecific targeting (Chames & Baty, 2009, mAbs 1[6]:1-9; Holliger & Hudson, 2005, Nature Biotechnology 23[9]:1126-1136).

Initially, bispecific antibodies were made by fusing two cell lines that each produced a single monoclonal antibody (Milstein et al., 1983, Nature 305:537-540). Although the resulting hybrid hybridoma or quadroma did produce bispecific antibodies, they were only a minor population, and extensive purification was required to isolate the desired antibody. An engineering solution to this was the use of antibody fragments to make bispecifics. Because the considerable diversity of the antibody variable region (Fv) makes it possible to produce an Fv that recognizes virtually any antigen or epitope, the typical approach to multispecifics generation is the introduction of new variable regions, either in the in the context of a “native” full-length immunoglobulin-like molecules (e.g., as an IgG), antibody fragments (e.g., single-chain variable fragments (scFvs), tandem scFvs, Fabs, diabodies, chain diabodies, Fab₂ bispecifics and the like; see, e.g., Chames et al., Br. J. Pharmacol, Vol. 157(2):220-233 (2009)), or non-native formats including such fragments. Because such fragments lack the complex quaternary structure of a full length antibody, variable light and heavy chains can be linked in single genetic constructs. While these formats can often be expressed at high levels in bacteria and may have favorable penetration benefits due to their small size, they clear rapidly in vivo and can present manufacturing obstacles related to their production and stability. A principal cause of these drawbacks is that antibody fragments typically lack the constant region of the antibody with its associated functional properties, including larger size, high stability, and binding to various Fc receptors and ligands that maintain long half-life in serum (i.e. the neonatal Fc receptor FcRn) or serve as binding sites for purification (i.e. protein A and protein G).

More recent work has attempted to address the shortcomings of fragment-based bispecifics by engineering dual binding into full length antibody-like formats (Wu et al., 2007, Nature Biotechnology 25[11]:1290-1297; U.S. Ser. No. 12/477,711; Michaelson et al., 2009, mAbs 1[2]:128-141; PCT/US2008/074693; Zuo et al., 2000, Protein Engineering 13[5]:361-367; U.S. Ser. No. 09/865,198; Shen et al., 2006, J Biol Chem 281[16]:10706-10714; Lu et al., 2005, J Biol Chem 280[20]:19665-19672; PCT/US2005/025472). These formats overcome some of the obstacles of the antibody fragment bispecifics, principally because they contain an Fc region. One significant drawback of these formats is that, because they build new antigen binding sites on top of the homodimeric constant chains, binding to the new antigen is always bivalent.

For many antigens that are attractive as co-targets in a therapeutic bispecific format, the desired binding is monovalent rather than bivalent. For example, for many immune receptors, cellular activation is accomplished by cross-linking of a monovalent binding interaction. The mechanism of cross-linking is typically mediated by antibody/antigen immune complexes, or via effector cell to target cell engagement. For example, the low affinity Fc gamma receptors (FcγRs) such as FcγRIIa, FcγRIIb, and FcγRIIIa bind monovalently to the antibody Fc region. Monovalent binding does not activate cells expressing these FcγRs; however, upon immune complexation or cell-to-cell contact, receptors are cross-linked and clustered on the cell surface, leading to activation. For receptors responsible for mediating cellular killing, for example FcγRIIIa on natural killer (NK) cells, receptor cross-linking and cellular activation occurs when the effector cell engages the target cell in a highly avid format (Bowles & Weiner, 2005, J Immunol Methods 304:88-99). Similarly, on B cells the inhibitory receptor FcγRIIb down-regulates B cell activation only when it engages into an immune complex with the cell surface B-cell receptor (BCR), a mechanism that is mediated by immune complexation of soluble IgGs with the same antigen that is recognized by the BCR (Heyman 2003, Immunol Lett 88[2]:157-161; Smith and Clatworthy, 2010, Nature Reviews Immunology 10:328-343). As another example, CD3 activation of T-cells occurs only when its associated T-cell receptor (TCR) engages antigen-loaded MHC on antigen presenting cells in a highly avid cell-to-cell synapse (Kuhns et al., 2006, Immunity 24:133-139). Indeed nonspecific bivalent cross-linking of CD3 using an anti-CD3 antibody elicits a cytokine storm and toxicity (Perruche et al., 2009, J Immunol 183[2]:953-61; Chatenoud & Bluestone, 2007, Nature Reviews Immunology 7:622-632). Thus for practical clinical use, the preferred mode of CD3 co-engagement for redirected killing of targets cells is monovalent binding that results in activation only upon engagement with the co-engaged target. Thus while bispecifics generated from antibody fragments suffer biophysical and pharmacokinetic hurdles, a drawback of those built with full length antibody-like formats is that they engage co-target antigens multivalently in the absence of the primary target antigen, leading to nonspecific activation and potentially toxicity.

Certain bispecific antibodies have been reported that can co-engage distinct target antigens (see, e.g., US 2011/0054151). The formats disclosed therein comprise two distinct polypeptides, wherein one polypeptide is provided in an “N-terminal VH-1-Fc region-C-terminal VH-2” orientation and the second polypeptide is provided in an “N-terminal VL-1-Fc region-C-terminal VL-2” orientation. However, as a result of the high degree of homology in, e.g., VH and VL framework regions that are disclosed in US 2011/0054151, nucleic acids and vectors encoding such polypeptides are susceptible to undesirable homologous recombination events when introduced into host cells (such as yeast cells), in which internal nucleic acid regions that flanked by highly homologous nucleic acid regions (e.g., framework regions of VLs of VHs) are excised (“looped out”) as a consequence of the homologous recombination event (see, e.g., FIG. 2 herein). Such undesirable homologous recombination events can give rise to generation of undesired by-products that are expressed from vectors that have undergone the homologous recombination event described above and represented in FIG. 2 herein, as well as relatively low expression levels of the desired multispecific antibody analog product. The suboptimal expression levels of the desired product and sample heterogeneity that results from co-expression of both the designed nucleic acids and those that have been “looped out” require laborious, time- and resource-intensive purification schemes in order to isolate the desired multivalent antibody analogs.

Additionally the antibody analogs disclosed in, for example US 2011/0054151 are limited to the “[scFv-Fc]×2”, “[scFv-Fc][empty-Fc]”, “[empty-Fc]x2”mAB-Fv”, “mAb-Fab”, “Fab-Fv” and “Fab-Fab” formats disclosed therein (see US 2011/0054151 FIGS. 1 and 8), and thus fail to teach formats in which, for example, a multivalent antibody analog comprises: one or more N-terminal antigen binding sites, such as one or more Fabs and/or one or more Fvs; and one or more single chain antigen binding sites, such as an scFv, covalently attached to the C-terminus of an Fc-region or a CH3 domain of the multivalent antibody analog. Such formats in which one or more antigen binding sites comprise a C-terminal single chain antigen binding moiety, such as an scFv, would afford advantages, such as: enhanced flexibility with regard to antigen binding site orientation and attachment point relative to the overall antibody analog architecture; and greater number of antigen binding sites that may be included in a given multivalent antibody analog. Such advantages would provide the artisan with greater flexibility in developing therapeutic and diagnostic multivalent antibody analogs suitable for addressing complex biological processes and associated disease states that require the ability to co-engage multiple epitope and targets.

There remains, therefore a need for multivalent antibody analogs that, for example, minimize some of the expression and production shortcomings described above and herein throughout. There also remains a need for multivalent and multispecific antibody analogs that allow for greater flexibility in the number, orientation, and attachment points antigen binding sites in the context of a multivalent antibody analog format.

### SUMMARY OF THE INVENTION

The present invention provides, inter alia, multivalent antibody analogs (referred to interchangeably throughout as “analogs” or “antibody analogs”, including bivalent, trivalent, tetravalent, pentavalent antibody analogs, and the like, which advantageously co-engage at least two different antigens or epitopes (also referred to “targets”, used interchangeably throughout), such antigens comprising different epitopes present on the same target moiety or on different target moieties, methods for their preparation, and methods of their use. Accordingly, the multivalent antibody analogs may also be multispecific, for example, bispecific, trispecific, tetraspecific, pentaspecfic, and the like.

In certain embodiments, the invention provides multivalent antibody analogs comprising a first polypeptide and a second polypeptide, wherein: a) the first polypeptide comprises a first heavy chain comprising a variable heavy region, a CH2 domain or a variant thereof, and a CH3 domain or a variant thereof; wherein the C-terminus of the CH3 domain or variant thereof is covalently attached to a first variable light domain (V_{L}); and b) the second polypeptide comprises a first light chain covalently attached to the N-terminus of an Fc region of a heavy chain, wherein the Fc region comprises a CH2 domain or a variant thereof and a CH3 domain or a variant thereof, and wherein the CH3 domain or variant thereof is covalently attached to a first variable heavy domain (V_{H}); wherein said first heavy chain and said first light chain form a first antigen binding site and said first V_{L} and said first V_{H} form a second antigen binding site.

In certain other embodiments, the invention provides multivalent antibody analogs comprising a first polypeptide and a second polypeptide, wherein: a) the first polypeptide comprises a first heavy chain comprising a variable heavy region, a CH2 domain or a variant thereof, and a CH3 domain or a variant thereof; b) the second polypeptide comprises a first light chain covalently attached to the N-terminus of an Fc region of a heavy chain, wherein the Fc region comprises a CH2 domain or a variant thereof and a CH3 domain or a variant thereof; and c) either the first polypeptide or the second polypeptide further comprises a single chain antigen binding site, such as a single chain variable region (scFv), comprising a first VL that is covalently attached to a first VH, wherein the scFv is covalently attached to the CH3 domain or variant thereof of said first polypeptide or said second polypeptide; wherein said first heavy chain and said first light chain form a first antigen binding site and the first VL and the first VH form a second antigen binding site. In certain embodiments, such multivalent antibody analogs comprise an scFv in which: the first VL is attached to the CH3 domain, or variant thereof, of the first heavy chain via a linker moiety; the first VL is attached to the CH3 domain, or variant thereof, of the Fc region via a linker moiety; the first VH is attached to the CH3 domain, or variant thereof, of the first heavy chain via a linker moiety; or the first VH is attached to the CH3 domain, or variant thereof, of the Fc region via a linker moiety. In certain embodiments, such multivalent antibody analogs comprise an scFv in which: the second VL is attached to the CH3 domain, or variant thereof, of the first heavy chain via a linker moiety; the second VL is attached to the CH3 domain, or variant thereof, of the Fc region via a linker moiety; the second VH is attached to the CH3 domain, or variant thereof, of the first heavy chain via a linker moiety; or the second VH is attached to the CH3 domain, or variant thereof, of the Fc region via a linker moiety.

In yet other embodiments, the invention provides multivalent antibody analogs comprising a first polypeptide and a second polypeptide, wherein: a) the first polypeptide comprises a first heavy chain comprising a variable heavy region, a CH2 domain or a variant thereof, and a CH3 domain or a variant thereof; b) the second polypeptide comprises a first light chain covalently attached to the N-terminus of an Fc region of a heavy chain, wherein the Fc region comprises a CH2 domain or a variant thereof and a CH3 domain or a variant thereof; and c) the first polypeptide further comprises a single chain variable region (scFv) comprising a first VL that is covalently attached to a first VH; and the second polypeptide further comprises a single chain variable region (scFv) comprising a second VL that is covalently attached to a second VH; wherein one scFv is covalently attached to the CH3 domain of variant thereof of said first polypeptide and the other scFv is covalently attached to the CH3 domain or variant thereof of said second polypeptide; wherein said first heavy chain and said first light chain form a first antigen binding site, the first VL and the first VH form a second antigen binding site, and the second VL and the second VH form a third antigen binding site.

In certain embodiments, the first polypeptide or the inventive multivalent antibody analogs further comprise a CH1 domain or a variant thereof covalently attached to the CH2 domain or a variant thereof; and the first light chain of the second polypeptide of the inventive antibody analogs further comprise either a Vkappa domain or a Vlambda domain covalently attached to C-terminus of the VL domain and to the N-terminus of an Fc region of the heavy chain.

In further embodiments, the first polypeptide and the second polypeptide of the inventive multivalent antibody analogs each further comprises a hinge region, and wherein the hinge regions each contain at least one thiol group that is capable of participating in an intermolecular disulfide bond such that the first and the second polypeptide are covalently linked as a result of formation of the disulfide bond. In some embodiments the thiol group is provided by a cysteine residue.

In other embodiments the first VL of the inventive multivalent antibody analogs is covalently attached to the CH3 domain, or variant thereof, of the first heavy chain via a linker moiety. In certain embodiments the first VH is covalently attached to the CH3 domain, or variant thereof, of the Fc region of the inventive multivalent antibody analog via a linker moiety.

In further embodiments, the multivalent antibody analogs further comprise a third antigen binding site. The third antigen binding site may be covalently attached via a linker moiety to either: the first VL; or the first VH. In still further embodiments of such multivalent antibody analogs, the third antigen binding site comprises a single chain antigen binding site, such as a single chain variable region (scFv), wherein the scFv comprises a second VL that is covalently attached to a second VH. In certain embodiments, the second VL is covalently attached to the second VH via a linker moiety. In certain embodiments, the second VL is attached to the first VL via a linker moiety; the second VH is attached to the first VH via a linker moiety; the second VL is attached to the first VH via a linker moiety; or the second VH is attached to the first VL via the third linker moiety.

In yet further embodiments the inventive multivalent antibody analogs further comprise a fourth antigen binding site. In certain of these embodiments, the fourth antigen binding site is covalently attached either: the first VL via a linker moiety; or the first VH via a linker moiety. The certain embodiments, the fourth antigen binding site may comprise a second single chain variable region (scFv), wherein said second scFv comprises a third VL that is covalently attached to a third VH. In certain of these embodiments the third VL may be covalently attached to the third VH via a linker moiety. In certain embodiments, the third VL is attached to the first VL via a linker moiety; the third VH is attached to the first VH via a linker moiety; the second VL is attached to the first VH via a linker moiety; or the second VH is attached to the first VL via a linker moiety.

In certain embodiments, one or more of the linker moieties the inventive multivalent antibody analogs comprises a peptide from 1 to 75 amino acids in length, inclusive. In certain embodiments, the linker moieties independently comprises at least one of the 20 naturally occurring amino acids. In further embodiments, one or more of the linker moieties independently comprises at least one non-natural amino acid incorporated by chemical synthesis, post-translational chemical modification or by in vivo incorporation by recombinant expression in a host cell. In particular embodiments, the one or more of the linker moieties independently comprises one or more amino acids selected from the group consisting of serine, glycine, alanine, proline, asparagine, glutamine, glutamate, aspartate, and lysine.

In certain embodiments, the one or more of the linker moieties independently comprises a majority of amino acids that are sterically unhindered. In other embodiments the one or more of the linker moieties independently comprises one or more of the following: an acidic linker, a basic linker, and a structural motif. In yet other embodiments, the one or more of the linker moieties independently comprises: polyglycine, polyalanine, poly(Gly-Ala), or poly(Gly-Ser). In still further embodiments, one or more of the linker moieties independently comprises: a polyglycine selected from the group consisting of: (Gly)₃, (Gly)₄ (SEQ ID NO: 1), and (Gly)₅ (SEQ ID NO: 2). In still further embodiments, one or more of the linker moieties independently comprises (Gly)₃Lys(Gly)₄ (SEQ ID NO: 3); (Gly)₃AsnGlySer(Gly)₂ (SEQ ID NO: 4); (Gly)₃Cys(Gly)₄ (SEQ ID NO: 5); and GlyProAsnGlyGly (SEQ ID NO: 24). In yet further embodiments, the one or more one or more of the linker moieties independently comprises a combination of Gly and Ala. In certain embodiments, the one or more of the linker moieties independently comprises a combination of Gly and Ser. In other embodiments, the one or more of the linker moieties independently comprises a combination of: Gly and Glu; or Gly and Asp. In other embodiments, the one or more of the linker moieties independently comprises a combination of Gly and Lys.

In particular embodiments, the inventive multivalent antibody analogs comprise one or more linker moieties, wherein the one or more linker moieties independently comprises a sequence selected from group consisting of: [Gly-Ser]ₙ (SEQ ID NO: 6); [Gly-Gly-Ser]ₙ (SEQ ID NO: 7); [Gly-Gly-Gly-Ser]ₙ (SEQ ID NO: 8); [Gly-Gly-Gly-Gly-Ser]ₙ (SEQ ID NO: 9); [Gly-Gly-Gly-Gly-Ser-Gly-Gly-Gly-Gly]ₙ (SEQ ID NO: 10); [Gly-Gly-Gly-Gly-Ser-Gly-Gly-Gly-Gly-Ser-Gly-Gly-Gly-Gly]ₙ (SEQ ID NO: 11); [Gly-Gly-Gly-Gly-Ser Gly-Gly-Gly-Gly-Ser-Gly-Gly-Gly-Gly-Ser-Gly-Gly-Gly-Gly]ₙ (SEQ ID NO: 12); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 13); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 14); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 15); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75. In other particular embodiments, the inventive multivalent antibody analogs comprise one or more linker moieties, wherein the one or more linker moieties independently comprises a sequence selected from group consisting of: [Gly-Glu]ₙ (SEQ ID NO: 16); [Gly-Gly-Glu]ₙ (SEQ ID NO: 17); [Gly-Gly-Gly-Glu]ₙ (SEQ ID NO: 18); [Gly-Gly-Gly-Gly-Glu]ₙ (SEQ ID NO: 19); [Gly-Asp]n (SEQ ID NO: 20); [Gly-Gly-Asp]ₙ (SEQ ID NO: 21); [Gly-Gly-Gly-Asp]ₙ (SEQ ID NO: 22); [Gly-Gly-Gly-Gly-Asp]ₙ (SEQ ID NO: 23); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

In yet further embodiments, the inventive multivalent antibody analogs according comprise at least one linker moiety, wherein the length of each at least one linker moiety is independently selected from 1 through 35 amino acids in length. In yet further embodiments, the inventive multivalent antibody analogs according comprise at least one linker moiety, wherein the length of each at least one linker moiety is independently selected from 5 through 35 amino acids in length. In still further embodiments, the inventive multivalent antibody analogs according comprise at least one linker moiety, wherein the length of each at least one linker moiety is independently selected from 10 through 35 amino acids in length. In other embodiments, the inventive multivalent antibody analogs according comprise at least one linker moiety, wherein the length of each at least one linker moiety is independently selected from 14 through 35 amino acids in length. In yet other embodiments, the inventive multivalent antibody analogs according comprise at least one linker moiety, wherein the length of each at least one linker moiety is independently selected from 19 through 35 amino acids in length.

In certain embodiments the inventive multivalent antibody analogs comprise either: a CH2 domain variant; a CH3 domain variant; or a CH2 domain variant and a CH3 domain variant; which independently comprises at least one different amino acid substitution such that a heterodimeric domain pair is generated such that heterodimerization of said variants is favored over homodimerization. In certain embodiments, either: a) the CH2 domain variant; the CH3 domain variant; or the CH2 domain variant and the CH3 domain variant; independently comprises a at least one protuberance in either the CH2 domain or the CH3 domain of the first polypeptide and at least one corresponding cavity in the CH2 domain or the CH3 domain of the second; or b) the CH2 domain variant; the CH3 domain variant; or the CH2 domain variant and the CH3 domain variant; independently comprises at least one cavity in either the CH2 domain or the CH3 domain of the first polypeptide and at least one corresponding protuberance in the CH2 domain or the CH3 domain of the second polypeptide. In certain other embodiments, either: a) the CH2 domain variant; the CH3 domain variant; or the CH2 domain variant and the CH3 domain variant; independently comprises at least one substituted negatively-charged amino acid in either the CH2 domain or the CH3 domain of the first polypeptide and at least one corresponding positively-charged amino acid in either the CH2 domain or the CH3 domain of the second polypeptide; or b) the CH2 domain variant; the CH3 domain variant; or the CH2 domain variant and the CH3 domain variant; independently comprises at least one substituted positively-charged amino acid in either the CH2 domain or the CH3 domain of the first polypeptide and at least one corresponding substituted negatively-charged substituted amino acid in either the CH2 domain or the CH3 domain of the second polypeptide.

In particular embodiments, the inventive multivalent analogs comprise at least one antigen binding site which comprises at least one humanized variable heavy domain or at least one humanized variable light domain. In other embodiments, the inventive multivalent antibody analogs comprise least one antigen binding site which comprises at least one complimentary determining region CDR that is derived from a non-human antibody or antibody fragment.

In certain embodiments, the inventive multivalent analogs comprise at least one antigen binding site binds an epitope from a tumor associated antigen, a hormone receptor, a cytokine receptor, chemokine receptor, a growth factor receptor, an immune activating receptor, a hormone, a cytokine, a chemokine, a growth factor, a G protein-coupled receptor, or a transmembrane receptor.

In certain other embodiments, the inventive multivalent analogs comprise at least one antigen binding site binds a target associated with an autoimmune disorder, an inflammatory disorder, an oncological disorder, neuromuscular disorder, a neurodegenerative disorder, a metabolic disorder, or an infectious disease.

In certain other embodiments, the inventive multivalent analogs bind at least two different targets.

In still other embodiments, the inventive multivalent analogs bind at least three different targets.

In yet other embodiments, the inventive multivalent analogs the antibody analog binds at least four different targets.

In other embodiments, the inventive multivalent analogs bind at least one target monovalently.

In further embodiments, the inventive multivalent analogs bind at least two targets monovalently.

In still further embodiments, the inventive multivalent analogs bind at least three targets monovalently.

In yet further embodiments, the inventive multivalent analogs bind at least four targets monovalently.

In certain embodiments, the inventive multivalent analogs are selected from the group consisting of the antibody analogs disclosed in the Examples.

Certain other embodiments provide methods of treating an autoimmune disorder, an inflammatory disorder, an oncological disorder, neuromuscular disorder, a neurodegenerative disorder, a metabolic disorder, or an infectious disease, the method comprising providing or administering a therapeutically effective amount of one or more of the multivalent antibody analogs.

Certain other embodiments provide methods of treating an autoimmune disorder, an inflammatory disorder, an oncological disorder, neuromuscular disorder, a neurodegenerative disorder, a metabolic disorder, or an infectious disease, the method comprising providing or administering a therapeutically effective amount of a multivalent antibody analog selected from the group consisting of the antibody analogs disclosed in the Examples.

### BRIEF DESCRIPTION OF THE FIGURES

FIGS. 1A through 1M provide schematic representations of exemplary multivalent antibody analogs as described in the Examples. As described throughout: “VH” means variable heavy domain; “VL” means variable light domain; “CL” means constant light domain (or constant light chain domain); “CH1” means constant heavy domain 1; “CH2” means constant heavy domain 2; “CH3” means constant heavy domain 3; “—S—S—” means a disulfide bond; lines represent linkers. FIG. 1A: representation of an bivalent antibody analog, which may comprise a bispecific antibody analog, comprising two polypeptides, wherein a first polypeptide is generated in the N-terminal VH-1-Fc region-C-terminal VL-2 orientation and a second polypeptide is generated in the N-terminal VL-1-C-terminal VH-2 orientation, as described throughout. FIG. 1B: representation of a tetravalent antibody analog, which may comprise a bispecific antibody analog, a trispecific antibody analog, or a tetraspecific antibody analog, wherein: a first polypeptide is generated in the N-terminal VH-1-Fc region-C-terminal VL-2 orientation and further comprises an scFv covalently attached to the C-terminal VL-2 via the VH of the scFv; and a second polypeptide is generated in the N-terminal VL-1-C-terminal VH-2 orientation and further comprises an scFv covalently attached to the C-terminal VL-2 via the VH of the scFv; as described throughout. FIG. 1C: representation of a tetravalent antibody analog, which may comprise a bispecific antibody analog, a trispecific antibody analog, or a tetraspecific antibody analog, wherein: a first polypeptide is generated in the N-terminal VH-1-Fc region-C-terminal VL-2 orientation and further comprises an scFv covalently attached to the C-terminal VL-2 via the VL of the scFv; and a second polypeptide is generated in the N-terminal VL-1-C-terminal VH-2 orientation and further comprises an scFv covalently attached to the C-terminal VH-2 via the VL of the scFv; as described throughout. FIG. 1D: representation of a tetravalent antibody analog, which may comprise a bispecific antibody analog, a trispecific antibody analog, or a tetraspecific antibody analog, wherein: a first polypeptide is generated in the N-terminal VH-1-Fc region-C-terminal VL-2 orientation and further comprises an scFv covalently attached to the C-terminal VL-1 via the VH of the scFv; and a second polypeptide is generated in the N-terminal VL-1-C-terminal VH-2 orientation and further comprises an scFv covalently attached to the C-terminal VH-2 via the VH of the scFv; as described throughout. FIG. 1E: representation of a tetravalent antibody analog, which may comprise a bispecific antibody analog, a trispecific antibody analog, or a tetraspecific antibody analog, wherein: a first polypeptide is generated in the N-terminal VH-1-Fc region-C-terminal VL-2 orientation and further comprises an scFv covalently attached to the C-terminal VL-2 via the VH of the scFv; and a second polypeptide is generated in the N-terminal VL-1-C-terminal VH-2 orientation and further comprises an scFv covalently attached to the C-terminal VH-2 via the VL of the scFv; as described throughout. FIG. 1F: representation of a bivalent antibody analog, which may comprise a bispecific antibody analog, wherein: a first polypeptide is generated in the N-terminal VH-1-C-terminal Fc region orientation; and a second polypeptide is generated in the N-terminal VL-1-Fc region-C-terminal scFv orientation, wherein the scFv is covalently attached to the C-terminal CH3 via the VL of the scFv; as described throughout. FIG. 1G: representation of a bivalent antibody analog, which may comprise a bispecific antibody analog, wherein: a first polypeptide is generated in the N-terminal VH-1-Fc region-C-terminal scFv orientation, wherein the scFv is covalently attached to the C-terminal CH3 via the VL of the scFv; and a second polypeptide is generated in the N-terminal VL-1-C-terminal Fc region orientation; as described throughout. FIG. 1H: representation of a bivalent antibody analog, which may comprise a bispecific antibody analog, wherein: a first polypeptide is generated in the N-terminal VH-1-C-terminal Fc region orientation; and a second polypeptide is generated in the N-terminal VL-1-Fc region-C-terminal scFv orientation, wherein the scFv is covalently attached to the C-terminal CH3 via the VH of the scFv; as described throughout. FIG. 1I: representation of a bivalent antibody analog, which may comprise a bispecific antibody analog, wherein: a first polypeptide is generated in the N-terminal VH-1-Fc region-C-terminal scFv orientation wherein the scFv is covalently attached to the C-terminal CH3 via the VH of the scFv; and a second polypeptide is generated in the N-terminal VL-1-C-terminal Fc region orientation; as described throughout. FIG. 1I: representation of a trivalent antibody analog, which may comprise a bispecific antibody analog or a trispecific antibody analog, wherein: a first polypeptide is generated in the N-terminal VH-1-C-terminal Fc region orientation and further comprises an scFv covalently attached to the C-terminal CH3 via the VH of the scFv; and a second polypeptide is generated in the N-terminal VL-1-C-terminal Fc region orientation and further comprises an scFv covalently attached to the C-terminal CH3 via the VH of the scFv; as described throughout. FIG. 1K: representation of a trivalent antibody analog, which may comprise a bispecific antibody analog or a trispecific antibody analog, wherein: a first polypeptide is generated in the N-terminal VH-1-C-terminal Fc region orientation and further comprises an scFv covalently attached to the C-terminal CH3 via the VL of the scFv; and a second polypeptide is generated in the N-terminal VL-1-C-terminal Fc region orientation and further comprises an scFv covalently attached to the C-terminal CH3 via the VL of the scFv; as described throughout. FIG. 1L: representation of a trivalent antibody analog, which may comprise a bispecific antibody analog or a trispecific antibody analog, wherein: a first polypeptide is generated in the N-terminal VH-1-C-terminal Fc region orientation and further comprises an scFv covalently attached to the C-terminal CH3 via the VH of the scFv; and a second polypeptide is generated in the N-terminal VL-1-C-terminal Fc region orientation and further comprises an scFv covalently attached to the C-terminal CH3 via the VL of the scFv; as described throughout. FIG. 1M: representation of a trivalent antibody analog, which may comprise a bispecific antibody analog or a trispecific antibody analog, wherein: a first polypeptide is generated in the N-terminal VH-1-C-terminal Fc region orientation and further comprises an scFv covalently attached to the C-terminal CH3 via the VL of the scFv; and a second polypeptide is generated in the N-terminal VL-1-C-terminal Fc region orientation and further comprises an scFv covalently attached to the C-terminal CH3 via the VH of the scFv; as described throughout.

FIG. 2 provides a schematic representation of a homologous recombination event in which an internal region of a nucleic acid encoding a desired polypeptide product that is flanked by nucleic acid regions of high homology (e.g., the VH-1 and VH-2 regions in the Figure) is excised, giving rise to an undesired polypeptide product that is expressed from the recombined nucleic acid.

FIGS. 3A through 3D provide schematic representations of construction of vectors, and multivalent antibody analogs that are expressed thereby, as described in the Examples.

FIG. 4 depicts size exclusion chromatographic analysis of an exemplary bivalent antibody analog comprising an N-terminal Fab that binds Target A and a C-terminal Fv that binds Target B, as described in the Examples.

FIG. 5 depicts an assessment of individual monovalent binding and affinity measurements for an exemplary bivalent antibody analog comprising an N-terminal Fab that binds Target A and a C-terminal Fv that binds Target B, employing the two illustrated formats, as described in the Examples.

FIG. 6 depicts an assessment of simultaneous binding of an exemplary bivalent antibody analog designed to bind to Target A and Target B, employing the two illustrated formats, as described in the Examples.

FIG. 7 provides PAGE gel analysis of five different exemplary bivalent antibody analogs that bind Target A and Target B. Each analog was run under both reducing and non-reducing conditions, as indicated.

FIG. 8 depicts size exclusion chromatographic analysis of an exemplary bivalent antibody analog comprising an N-terminal Fab that binds Target A and a C-terminal scFv that binds Target B, as described in the Examples.

FIG. 9 depicts an assessment of individual monovalent binding and affinity measurements for an exemplary bivalent antibody analog comprising an N-terminal Fab that binds Target A and a C-terminal scFv that binds Target B, employing the two illustrated formats, as described in the Examples.

FIG. 10 depicts an assessment of simultaneous binding of an exemplary bivalent antibody analog designed to bind to Target A and Target B, employing the two illustrated formats, as described in the Examples.

### DETAILED DESCRIPTION OF THE INVENTION

The present invention provides, inter alia, multivalent antibody analogs (referred to interchangeably throughout as “analogs” or “antibody analogs”, including bivalent, trivalent, tetravalent, pentavalent antibody analogs, and the like, which advantageously co-engage at least two different antigens or epitopes (also referred to “targets”, used interchangeably throughout), such antigens comprising different epitopes present on the same target moiety or on different target moieties, methods for their preparation, and methods of their use. Accordingly, the multivalent antibody analogs may also be multispecific, for example, bispecific, trispecific, tetraspecific, pentaspecfic, and the like. It will be appreciated that certain non-limiting embodiments of the invention are herein throughout.

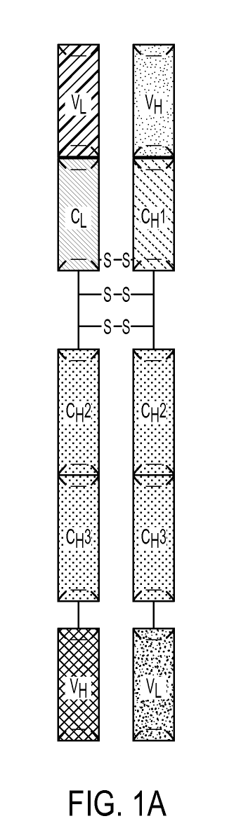

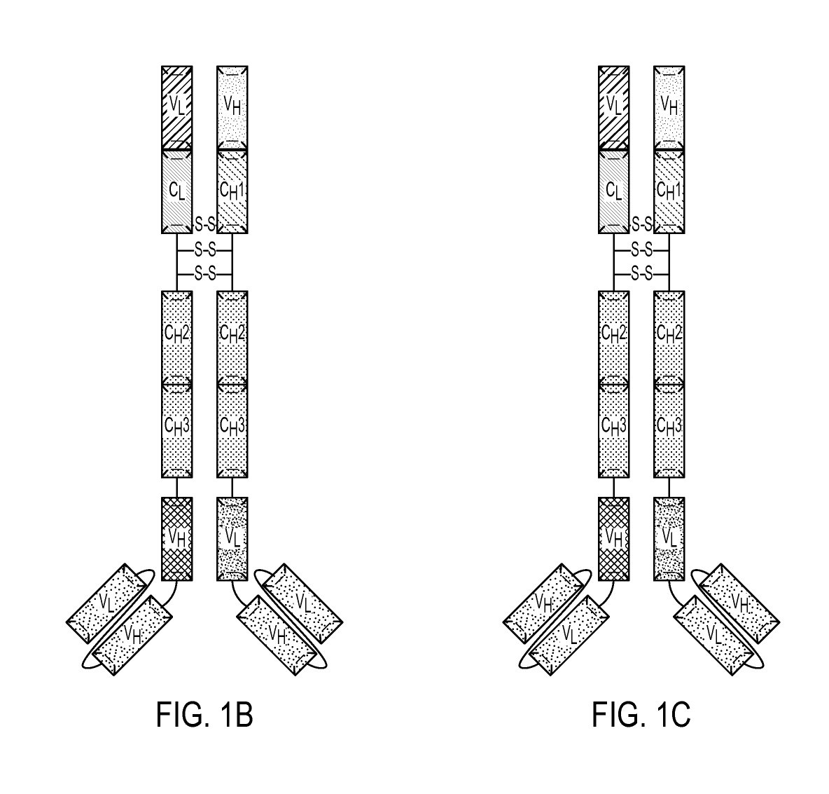

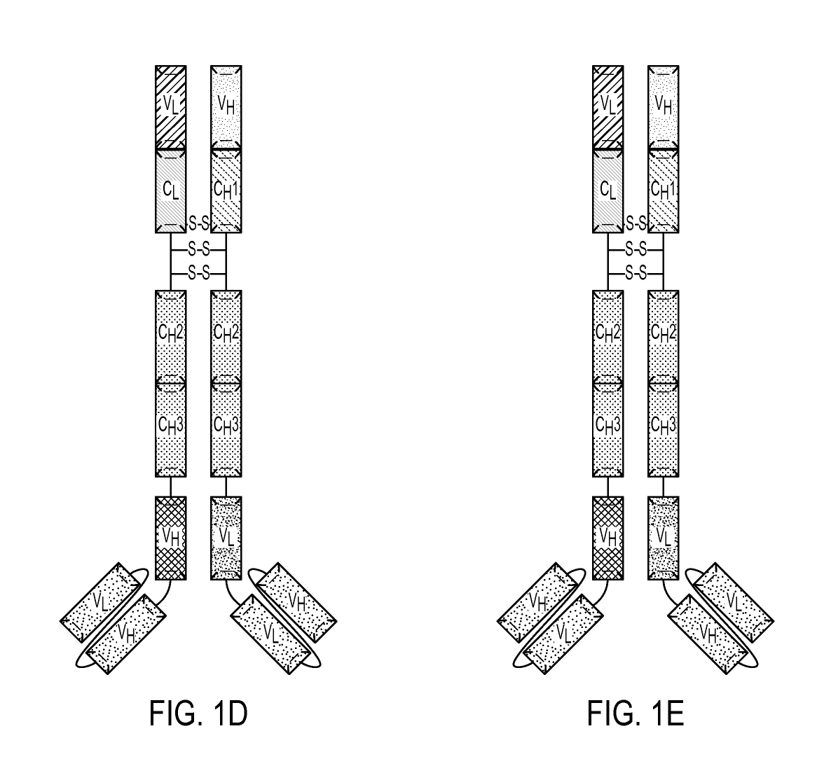

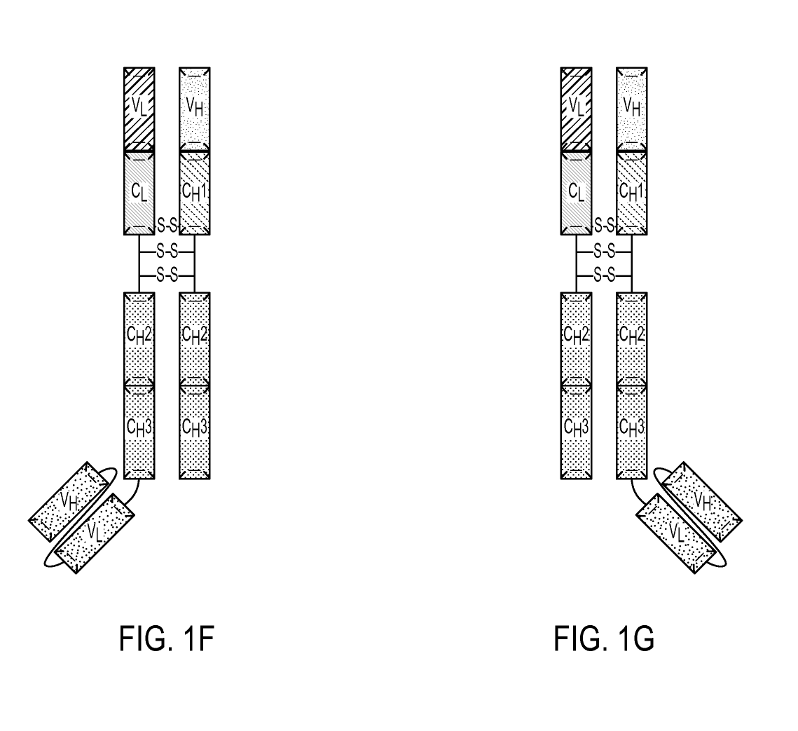

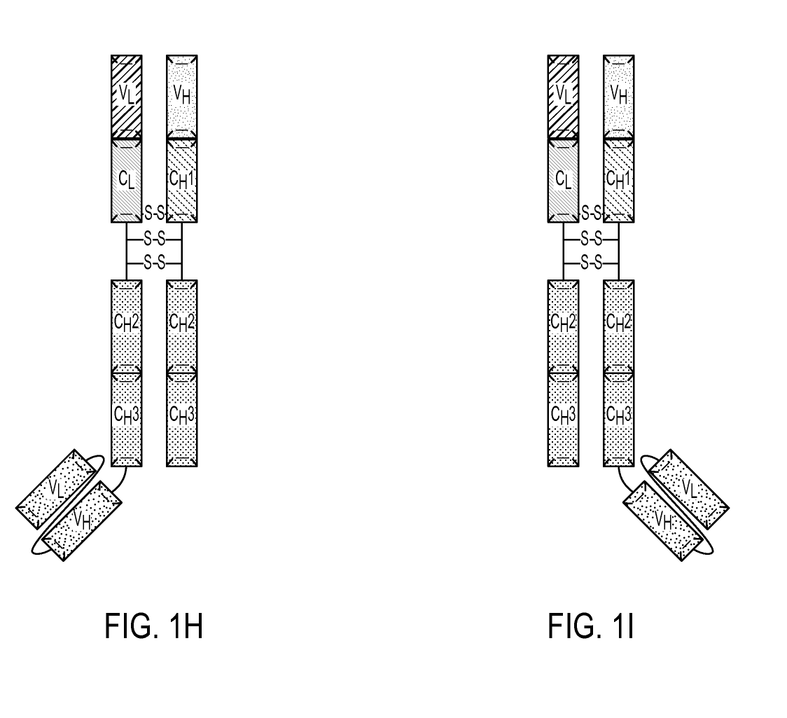

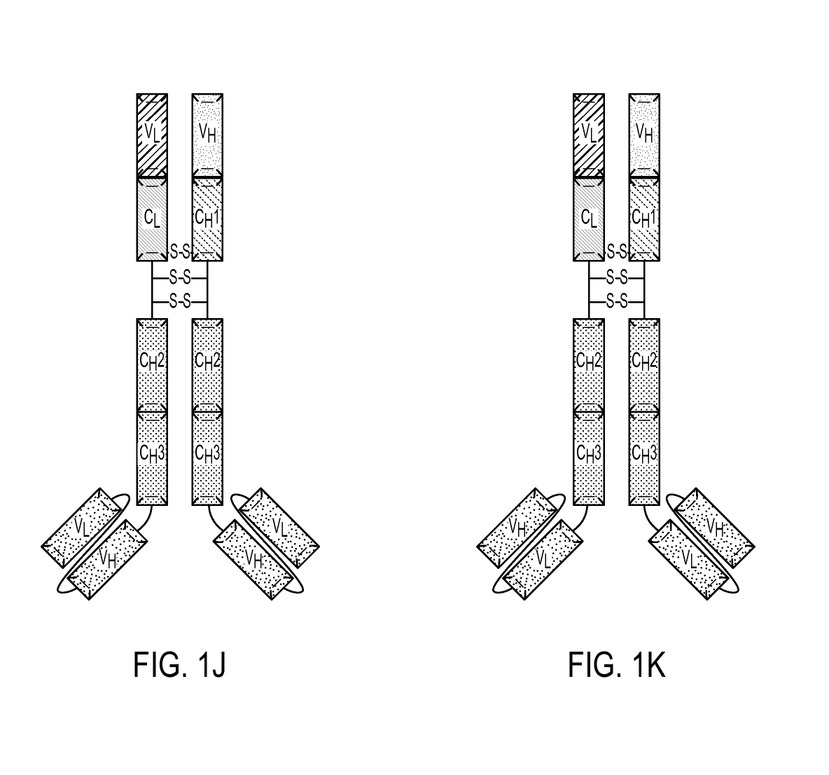

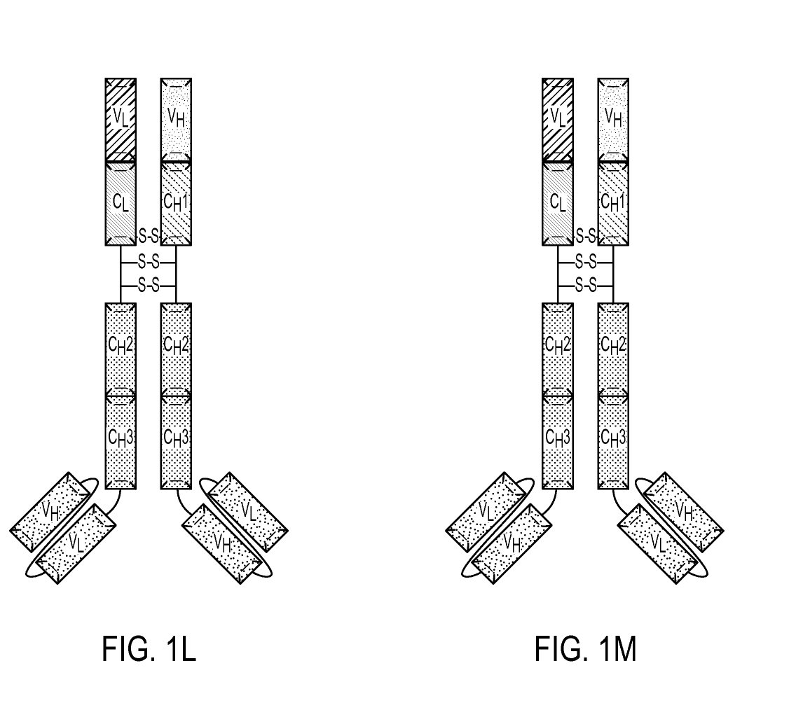

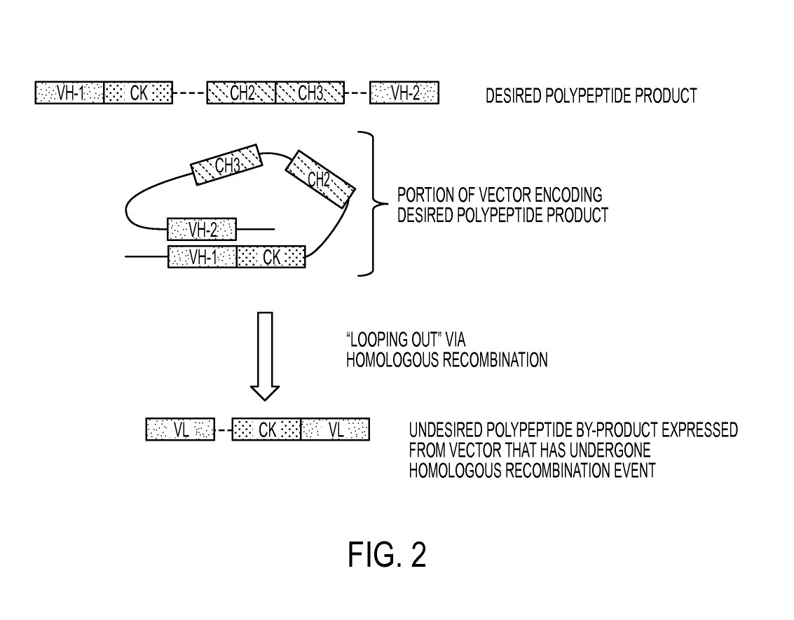

Certain of the inventive multivalent antibody analogs disclosed herein comprise a first polypeptide and a second polypeptide, wherein: a) the first polypeptide comprises a first heavy chain comprising a variable heavy region, a CH2 domain or a variant thereof, and a CH3 domain or a variant thereof; wherein the C-terminus of the CH3 domain or variant thereof is covalently attached to a first variable light domain (V_{L}); and b) the second polypeptide comprises a first light chain covalently attached to the N-terminus of an Fc region of a heavy chain, wherein the Fc region comprises a CH2 domain or a variant thereof and a CH3 domain or a variant thereof, and wherein the CH3 domain or variant thereof is covalently attached to a first variable heavy domain (V_{H}); wherein said first heavy chain and said first light chain form a first antigen binding site and said first V_{L} and said first V_{H} form a second antigen binding site. Advantageously, such analogs comprise first and second polypeptides that are prepared in “N-terminal VH to C-terminal VL” and “N-terminal VL to C-terminal VH” orientations, respectively, as illustrated, for example, in FIG. 1A herein. Nucleic acids encoding such polypeptides are significantly less susceptible to undesirable homologous recombination events when introduced into host cells (such as yeast cells, bacterial cells, and mammalian cells), in which internal nucleic acid regions that flanked by highly homologous nucleic acid regions (e.g., framework regions of VLs of VHs) are excised (“looped out”) as a consequence of the homologous recombination event (see, e.g., FIG. 2 herein). Such undesirable homologous recombination events can give rise to generation of undesired by-products that are expressed from vectors that have undergone the homologous recombination event described above and represented in FIG. 2 herein, as well as relatively low expression levels of the desired multispecific antibody analog product. The suboptimal expression levels of the desired product and sample heterogeneity that results from co-expression of both the designed nucleic acids and those that have been “looped out” require laborious, time- and resource-intensive purification schemes in order to isolate the desired multivalent antibody analogs. Accordingly, the disclosed analogs, and nucleic acids encoding such analogs, advantageously afford, e.g., improved expression and yield of the desired product, minimization of undesired by-products and sample heterogeneity, and simplifies or streamlined manufacturing requirements.

Certain other inventive analogs disclosed herein comprise a first polypeptide and a second polypeptide, wherein: a) the first polypeptide comprises a first heavy chain comprising a variable heavy region, a CH2 domain or a variant thereof, and a CH3 domain or a variant thereof; b) the second polypeptide comprises a first light chain covalently attached to the N-terminus of an Fc region of a heavy chain, wherein the Fc region comprises a CH2 domain or a variant thereof and a CH3 domain or a variant thereof; and c) either the first polypeptide or the second polypeptide further comprises a single chain antigen binding site, such as variable region (scFv) comprising a first VL that is covalently attached to a first VH, wherein said scFv is covalently attached to the CH3 domain or variant thereof of said first polypeptide or said second polypeptide; wherein said first heavy chain and said first light chain form a first antigen binding site and the first VL and the first VH form a second antigen binding site. Such formats in which one or more antigen binding sites comprise a C-terminal single chain antigen binding moiety, such as an scFv, would afford advantages, such as: enhanced flexibility with regard to antigen binding site orientation and attachment point relative to the overall antibody analog architecture; and greater number of antigen binding sites that may be included in a given multivalent antibody analog. Such advantages would provide the artisan with greater flexibility in developing therapeutic and diagnostic multivalent antibody analogs suitable for addressing complex biological processes and associated disease states that require the ability to co-engage multiple epitope and targets.

By “scFv” as used herein is meant a polypeptide consisting of two variable regions connected by a linker sequence; e.g., V_{H}-linker-VL, V_{H}-linker-VL, V_{κ}-linker-VL, or VL-linker-V_{H}. “Linkers” (also referred to a “linker moieties”, used interchangeably throughout), are described in more detail below.

By “Fab” or “Fab region” as used herein is meant the polypeptides that comprise the VH, CH1, VL, and CL immunoglobulin domains. Typically, the VH and CH1 domains comprise one polypeptide and the VL and CL domains comprise another polypeptide, wherein the two polypeptides are linked to one another via at least one inter-polypeptide disulfide bond. Fab may refer to this region in isolation, or this region in the context of a full length antibody or antibody fragment.

By “protein” or “polypeptide” as used herein is meant at least two covalently attached amino acids, which includes proteins, polypeptides, oligopeptides and peptides. The protein may be made up of naturally occurring amino acids and peptide bonds, or synthetic peptidomimetic structures, i.e. “analogs”, such as peptides.

By “position” as used herein is meant a location in the sequence of a protein or nucleic acid. Protein positions may be numbered sequentially, or according to an established format, for example the Kabat index for antibody variable regions or the EU index for antibody constant regions. For example, position 297 is a position in the human antibody IgG1. Corresponding positions are determined as outlined above, generally through alignment with other parent sequences.

By “residue” as used herein is meant a position in a protein and its associated amino acid identity. For example, Asparagine 297 (also referred to as Asn297, also referred to as N297) is a residue in the human antibody IgG1. In some embodiments it can also refer to nucleic acid bases.

As would be understood by those of ordinary skill in the art, the term “antibody” is used herein in the broadest sense and specifically encompasses at least monoclonal antibodies, polyclonal antibodies, multi-specific antibodies (e.g., bispecific antibodies), chimeric antibodies, humanized antibodies, human antibodies, antibody fragments, and derivatives thereof. An antibody is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. An “antibody” also refers to an immunoglobulin molecule, a fragment of an immunoglobulin molecule, or a derivative thereof, which has the ability to specifically bind to an antigen, which may be, for example: a protein; a polypeptide; peptide; a hormone; a cytokine; a chemokine; a growth factor; a neurotransmitter; a carbohydrate-containing biological molecule; a lipid or fatty acid-containing biological molecule; or other biological molecule; via an epitope present on such antigen.

Antibodies (used interchangeably with “immunoglobulins, or “immunoglobulin molecules”) can be monomeric, dimeric, trimeric, tetrameric, pentameric, etc., and comprise a class of structurally related proteins consisting of two pairs of polypeptide chains: one pair of light chains (LC) and one pair of heavy chains (HC), all of which are inter-connected by disulfide bonds. The structure of immunoglobulins has been well characterized. See for instance Fundamental Immunology Ch. 7 (Paul, W., ed., 2nd ed. Raven Press, N.Y. (1989)).

Traditional natural antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. IgA has several subclasses, including but not limited to IgA1 and IgA2. Thus, “isotype” as used herein is meant any of the classes and subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE. The distinguishing features between these antibody classes are their constant regions, although subtler differences may exist in the variable region.

Each of the light and heavy chains is made up of two distinct regions, referred to as the variable and constant regions. The IgG heavy chain is composed of four immunoglobulin domains linked from N- to C-terminus in the order VH-CH1-CH2-CH3, referring to the “variable heavy domain” (also referred to as a “heavy chain variable domain”, used interchangeably throughout), heavy chain constant domain 1, heavy chain constant domain 2, and heavy chain constant domain 3 respectively (also referred to as VH-Cγ1-Cγ2-Cγ3, referring to the variable heavy domain, constant gamma 1 domain, constant gamma 2 domain, and constant gamma 3 domain respectively). The IgG light chain is composed of two immunoglobulin domains linked from N- to C-terminus in the order VL-CL, referring to the “variable light domain” (also referred to as a “light chain variable domain”, used interchangeably throughout) and the light chain constant domain respectively. The constant regions show less sequence diversity, and are responsible for binding a number of natural proteins to elicit important biochemical events. The structure that constitutes the natural biological form of an antibody, including the variable and constant regions, is referred to herein as a “full length antibody”. In most mammals, including humans and mice, the full length antibody of the IgG isotype is a tetramer and consists of two identical pairs of two immunoglobulin chains, each pair having one light chain and one heavy chain, each light chain comprising a VL and a CL, and each heavy chain comprising a VH, CH1, a CH2, and a CH3. In some mammals, for example in camels and llamas, IgG antibodies may consist of only two heavy chains, each heavy chain comprising a variable domain attached to the Fc region.

The heavy chain constant region typically is comprised of three domains, CH1, CH2, and CH3, and the CH1 and CH2 domains are connected by a hinge region. Each light chain typically is comprised of a light chain variable domain (abbreviated herein as “V_{L}” or “VL”) and a light chain constant domain. The V_{H} and V_{L} domains may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each V_{H} and V_{L} is typically composed of three CDRs and four FRs, arranged from amino-terminus to carboxy-terminus in the following order: FR1, CDR1, FR2, CDR2, FR3, CDR3, FR4. Typically, the numbering of amino acid residues in this region is performed by the method described in Kabat (see, e.g., Kabat et al, in “Sequences of Proteins of Immunological Interest,” 5^{th} Edition, U.S. Department of Health and Human Services, 1992). Using this numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of V_{H} CDR2 and inserted residues (for instance residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “variable”, “variable domain”, or “variable region” each interchangeably refers to the portions of the immunoglobulin domains that exhibit variability in their sequence and that are involved in determining the specificity and binding affinity of a particular antibody (i.e., the “variable domain(s)”). Variability is not evenly distributed throughout the variable domains of antibodies; it is concentrated in sub-domains of each of the heavy and light chain variable regions. These sub-domains are called “hypervariable” regions or “complementarity determining regions” (CDRs). The more conserved (i.e., non-hypervariable) portions of the variable domains are called the “framework” regions (FRM). The variable domains of naturally occurring heavy and light chains each comprise four FRM regions, largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding site (see Kabat et al. Sequences of Proteins of Immunological Interest, 5th Ed. Public Health Service, National Institutes of Health, Bethesda, Md., 1991, incorporated by reference in its entirety). The constant domains are not directly involved in antigen binding, but exhibit various effector functions, such as, for example, antibody-dependent, cell-mediated cytotoxicity and complement activation.

The term “framework region” refers to the art-recognized portions of an antibody variable region that exist between the more divergent (i.e., hypervariable) CDRs. Such framework regions are typically referred to as frameworks 1 through 4 (FRM1, FRM2, FRM3, and FRM4) and provide a scaffold for the presentation of the six CDRs (three from the heavy chain and three from the light chain) in three dimensional space, to form an antigen-binding surface. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. MoI. Biol., 1987, 196: 901; Chothia et al, Nature, 1989, 342: 877; Martin and Thornton, J. MoI. Biol, 1996, 263: 800. Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al, in “Sequences of Proteins of Immunological Interest,” 5^{th} Edition, U.S. Department of Health and Human Services, 1992). The Kabat numbering scheme is a widely adopted standard for numbering the amino acid residues of an antibody variable domain in a consistent manner. Additional structural considerations can also be used to determine the canonical structure of an antibody. For example, those differences not fully reflected by Kabat numbering can be described by the numbering system of Chothia et al and/or revealed by other techniques, for example, crystallography and two or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., and their implications for construing canonical aspects of antibody structure, are described in the literature.

A variable light chain (VL) and corresponding variable heavy domain (VH) of the inventive multivalent antibody analogs comprise a binding domain, also referred to interchangeably throughout as an “antigen binding site” that interacts with an antigen. Thus, a “first variable light domain” and a “first variable heavy domain” of the inventive multivalent antibody analogs together form a “first antigen binding site”. Similarly, a “second variable light domain” and a “second variable heavy domain” of the inventive multivalent antibody analogs together form a “second antigen binding site”. A “third variable light domain” and a “third variable heavy domain” of the inventive multivalent antibody analogs together form a “third antigen binding site”, and so on.

The antigen binding sites for use in accordance with the invention, including the VHs, VLs, and/or CDRs that comprise such, may be obtained or derived from any source of such, as will be understood by the artisan. Accordingly, such antigen binding sites, VHs, VLs, and/or CDRs may be obtained or derived from hybridoma cells that express antibodies against a target recognized by such; from B cells from immunized donors, which express antibodies against a target recognized by such; from B-cells that have been stimulated to express antibodies antibodies against a target recognized by such; and or from identification of antibodies or antibody fragments that have been identified by screening a library comprising a plurality of polynucleotides or polypeptides for antigen binding antibodies (or antigen binding fragments thereof). With regard to the design, preparation, display, and implementation of such libraries for use in identifying and obtaining antigen binding sites for use in accordance with the invention, see, e.g., WO 2009/036379; WO2012009568; WO2010105256; U.S. Pat. No. 8,258,082;U.S. Pat. No. 6,300,064; U.S. Pat. No. 6,696,248; U.S. Pat. No. 6,165,718; U.S. Pat. No. 6,500,644; U.S. Pat. No. 6,291,158; U.S. Pat. No. 6,291,159; U.S. Pat. No. 6,096,551; U.S. Pat. No. 6,368,805; U.S. Pat. No. 6,500,644; and the like.

Any one or more of the antigen binding sites, VHs, VLs, or CDRs, and combinations thereof, of the inventive multivalent antibody analogs, may comprise sequences from a variety of species. In some embodiments, such antigen binding sites, VHs, VLs, or CDRs, and combinations thereof may be obtained from a nonhuman source, including but not limited to mice, rats, rabbits, camels, llamas, and monkeys. In some embodiments, the scaffold and/or framework regions can be a mixture from different species. As such, a multivalent antibody analog in accordance with the invention may comprise a chimeric antibody and/or a humanized antibody. In general, both “chimeric antibodies” and “humanized antibodies” refer to antibodies in which regions from more than one species have been combined. For example, “chimeric antibodies” traditionally comprise variable region(s) from a mouse or other nonhuman species and the constant region(s) from a human.

“Humanized antibodies” generally refer to non-human antibodies that have had the variable-domain framework regions swapped for sequences found in human antibodies. Generally in a humanized antibody the entire antibody, except the CDRs, is encoded by a polynucleotide of human origin or is identical to such an antibody except within its CDRs. The CDRs, one, some, or all of which are encoded by nucleic acids originating in a non-human organism, are grafted into the framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536. “Backmutation” of selected acceptor framework residues to the corresponding donor residues is often required to regain affinity that is lost in the initial grafted construct (see, e.g., U.S. Pat. No. 5,693,762). The humanized antibody optimally also will comprise at least a portion of an immunoglobulin constant region, typically that of a human immunoglobulin, and thus will typically comprise a human Fc region. A variety of techniques and methods for humanizing, reshaping, and resurfacing non-human antibodies are well known in the art (See Tsurushita & Vasquez, 2004, Humanization of Monoclonal Antibodies, Molecular Biology of B Cells, 533-545, Elsevier Science (USA), and references cited therein). In certain variations, the immunogenicity of the antibody is reduced using a method described in Lazar et al., 2007, Mol Immunol 44:1986-1998 and U.S. Ser. No. 11/004,590, entitled “Methods of Generating Variant Proteins with Increased Host String Content and Compositions Thereof”, filed on Dec. 3, 2004.

Accordingly, any one or more of the antigen binding sites, or one or more VHs, VLs, CDRs, or combinations thereof, which comprise the inventive multivalent antibody analogs disclosed herein may be derived from a non-human species and/or result from humanization of a non-human antibody or antibody fragment. Such VHs, VLs, and/or CDRs obtained or derived from non-human species, when included in the inventive multivalent analogs disclosed herein, are referred to as “humanized” such regions and/or domains.

The inventive antibody analogs disclosed herein preferably comprise first and second polypeptides that each comprise a hinge region, wherein each hinge region comprises at least one thiol group that is capable of participating in an intermolecular disulfide bond such that the first and the second polypeptide are covalently linked as a result of formation of the disulfide bond. As is understood in the art, chemical modification may be introduced into (or onto) certain residues within such hinge regions which effect the introduction of such thiol groups for disulfide bond formation. Alternatively, the thiol groups may be provided by a cysteine residue that is present within the hinge region. Such cysteines may be provided by native hinge polypeptide sequence, or may be introduced by mutagenesis into nucleic acid encoding the hinge region. As used herein, a “hinge” or a “hinge region” of the inventive antibody analogs may comprise or constitute a “linker moiety”.

In certain embodiments, the inventive antibody analogs disclosed herein comprise one or more linkers or linker moieties. Such linkers or linker moieties may comprise a peptidic linker moiety or a non-peptidic linker moiety. The terms “linker” and “linker moiety” and the like, means a divalent species (-L-) covalently bonded in turn to a polypeptide having a valency available for bonding and to an amino acid that comprises the inventive multivalent antibody analogs, which amino acid has a valency available for bonding. The available bonding site may conveniently comprise a side chain of an amino acid (e.g., a lysine, cysteine, or aspartic acid side chain, and homologs thereof). In some embodiments, the available bonding site in the analog is the side chain of a lysine or a cysteine residue. In some embodiments, the available bonding site in the analog is the N-terminal amine of a polypeptide comprising the analog. In some embodiments, the available bonding site in the analog is the C-terminal carboxyl of a polypeptide comprising the analog. In some embodiments, the available bonding site in the analog is a backbone atom (e.g., a c-alpha carbon atom) of a polypeptide comprising the analog.

Preferably, a linker moiety is employed to covalently attach a VH or a VL to the C-terminus of a CH3 domain of an antibody analogs. A linker moiety may also be employed to covalently attach a first VH or a first VL to a second VH or a second VL, respectively. A linker moiety may also be employed to covalently attach a first VH or a first VL to a second VL or a second VH, respectively. A linker moiety may also be employed to covalently attach a VH of a single chain antigen binding site, such as an scFv, to the VL of such a single chain antigen binding site, and vice versa. A linker moiety may also be employed to attach the VH or the VL of such a single chain antigen binding site, such as an scFv, to a C-terminus of a CH3 domain or variant thereof. A linker moiety may also be employed to attach a VH to the N-terminus of a CL domain or to the N-terminus of a CH2. A linker moiety may also be employed to attach a VL to the N-terminus of a CL domain or to the N-terminus of a CH2 domain. As will be appreciated, combinations and/or multiples of the foregoing may be employed in order to prepare any of the multivalent antibody analogs disclosed herein, such that a plurality of antigen binding sites may be included in such analogs, optionally with a multiple of specificities. Accordingly, a multivalent antibody analog may be generated by employing one or more linkers to covalently attach one, two, three, four, five, six, seven, or more VLs, VHs, and/or single chain antigen binding sites, such as scFvs to the first polypeptide, the second polypeptide, a VH, or a VL attached to the first polypeptide or the second polypeptide, and the like, so as to generate an antibody analog having bi-, tri-, tetra-, pent-, hexa-, hepta-, or octa-valency, and so on, and/or bi-, tri-, tetra-, pent-, hexa-, hepta-, or octa-specificity, and so on.

Accordingly, in certain embodiments, the multivalent antibody analog comprises a first VL that is covalently attached to the CH3 domain, or variant thereof, of the first heavy chain of the analog via a linker moiety, forming the second antigen binding site. In additional embodiments, the multivalent antibody analog comprises a first VH that is covalently attached to the CH3 domain, or variant thereof, of the Fc region of the analog via a linker moiety, thereby forming the second antigen binding site.

In further embodiments, the multivalent antibody analog comprises a third antigen binding site, wherein the third antigen binding site is covalently attached via a linker moiety to either the first VL or the first VH. In still further embodiments, the third antigen binding site comprises a single chain antigen binding site, such single chain variable region (scFv), wherein the scFv comprises a second VL that is covalently attached to a second VH via a linker moiety or wherein the second VL is covalently attached to the second VH via a linker moiety.

In further embodiments, the inventive multivalent antibody analogs further comprise additional binding sites, such as a fourth antigen binding site, a fifth antigen binding site, a sixth antigen binding site, and so on, wherein one or more of which may comprise a single chain antigen binding site, such as an scFv, which are attached via linker moieties to the other VLs and/or VHs of the multivalent antibody analog.

In certain embodiments the linker moieties comprise amino acids that are selected from glycine, alanine, proline, asparagine, glutamine, lysine, aspartate, and glutamate. In a further embodiment the linker moiety is made up of a majority of amino acids that are sterically unhindered, such as glycine, alanine and/or serine. In certain embodiments the linker moiety is comprises a sequence selected from the group Gly-Serb (SEQ ID NO: 6); [Gly-Gly-Ser]ₙ (SEQ ID NO: 7); [Gly-Gly-Gly-Ser]ₙ (SEQ ID NO: 8); [Gly-Gly-Gly-Gly-Ser]ₙ (SEQ ID NO: 9); [Gly-Gly-Gly-Gly-Ser-Gly-Gly-Gly-Gly]ₙ (SEQ ID NO: 10); [Gly-Gly-Gly-Gly-Ser-Gly-Gly-Gly-Gly-Ser-Gly-Gly-Gly-Gly]ₙ (SEQ ID NO: 11); [Gly-Gly-Gly-Gly-Ser Gly-Gly-Gly-Gly-Ser-Gly-Gly-Gly-Gly-Ser-Gly-Gly-Gly-Gly]ₙ (SEQ ID NO: 12); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 13); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 14); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 15); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

Such linkers may comprise: an acidic linker, a basic linker, and a structural motif, or combinations thereof; a polyglycine, a polyalanine, poly(Gly-Ala), or poly(Gly-Ser); (Gly)3, (Gly)4 (SEQ ID NO: 1), or (Gly)5 (SEQ ID NO: 2); (Gly)₃Lys(Gly)₄ (SEQ ID NO: 3), (Gly)₃AsnGlySer(Gly)₂ (SEQ ID NO: 4), (Gly)₃Cys(Gly)₄ (SEQ ID NO: 5), or GlyProAsnGlyGly (SEQ ID NO: 24), [Gly-Ser]ₙ (SEQ ID NO: 6), [Gly-Gly-Ser]ₙ (SEQ ID NO: 7), [Gly-Gly-Gly-Ser]ₙ (SEQ ID NO: 8), [Gly-Gly-Gly-Gly-Ser]ₙ (SEQ ID NO: 9), [Gly-Gly-Gly-Gly-Ser-Gly-Gly-Gly-Gly]ₙ (SEQ ID NO: 10), [Gly-Gly-Gly-Gly-Ser-Gly-Gly-Gly-Gly-Ser-Gly-Gly-Gly-Gly]ₙ (SEQ ID NO: 11), [Gly-Gly-Gly-Gly-Ser Gly-Gly-Gly-Gly-Ser-Gly-Gly-Gly-Gly-Ser-Gly-Gly-Gly-Gly]ₙ (SEQ ID NO: 12), [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 13), [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 14), or [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 15); [Gly-Glu]ₙ (SEQ ID NO: 16), [Gly-Gly-Glu]ₙ (SEQ ID NO: 17), [Gly-Gly-Gly-Glu]ₙ (SEQ ID NO: 18), [Gly-Gly-Gly-Gly-Glu]ₙ (SEQ ID NO: 19), [Gly-Asp]ₙ (SEQ ID NO: 20); [Gly-Gly-Asp]ₙ (SEQ ID NO: 21), [Gly-Gly-Gly-Asp]ₙ (SEQ ID NO: 22), [Gly-Gly-Gly-Gly-Asp]ₙ (SEQ ID NO: 23); where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

In certain embodiments, charged linker moieties are employed. Such charges linker moieties may contain a significant number of acidic residues (e.g., Asp, Glu, and the like), or may contain a significant number of basis residues (e.g., Lys, Arg, and the like), such that the linker moiety has a pi lower than 7 or greater than 7, respectively. As understood by the artisan, and all other things being equal, the greater the relative amount of acidic or basic residues in a given linker moiety, the lower or higher, respectively, the pI of the linker moiety will be. Such linker moieties may impart advantages to the multivalent antibody analogs disclosed herein, such as improving solubility and/or stability characteristics of such polypeptides at a particular pH, such as a physiological pH (e.g., between H 7.2 and pH 7.6, inclusive), or a pH of a pharmaceutical composition comprising such analogs, as well as allowing for optimization of characteristics such as rotational and translational flexibility of the domains and/or regions of the analog that are attached via the linker moiety. Such characteristics may advantageously be optimized and tailored for any given multivalent analog by the artisan.

Additionally, linker moieties may be employed which possess certain structural motifs or characteristics, such as an alpha helix. For example, such a linker moiety may contain a sequence that is selected from the group consisting of [Glu-Ala-Ala-Ala-Lys]ₙ (SEQ ID NO: 33), where n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75: for example, [Glu-Ala-Ala-Ala-Lys]3 (SEQ ID NO: 34), [Glu-Ala-Ala-Ala-Lys]4 (SEQ ID NO: 35), or [Glu-Ala-Ala-Ala-Lys]₅ (SEQ ID NO: 36), and so on. In still further embodiments the each linker moiety employed in the disclosed multivalent antibody analogs independently comprises: polyglycine, polyalanine, poly(Gly-Ala), or poly(Gly-Ser), (Gly)3, (Gly)4 (SEQ ID NO: 1), and (Gly)5 (SEQ ID NO: 2), (Gly)₃Lys(Gly)₄ (SEQ ID NO: 3), (Gly)₃AsnGlySer(Gly)₂ (SEQ ID NO: 4), (Gly)₃Cys(Gly)₄ (SEQ ID NO: 5), and GlyProAsnGlyGly (SEQ ID NO: 24), a combination of Gly and Ala, a combination of Gly and Ser, a combination of, Gly and Glu, a combination of Gly and Asp, a combination of Gly and Lys, or combinations thereof.

For example, an “acidic linker” is a linker moiety that has a pi of less than 7; between 6 and 7, inclusive; between 5 and 6, inclusive; between 4 and 5, inclusive; between 3 and 4, inclusive; between 2 and 3, inclusive; or between 1 and 2, inclusive. Similarly, a “basic linker” is a linker moiety that has a pi of greater than 7; between 7 and 8, inclusive; between 8 and 9, inclusive; between 9 and 10, inclusive; between 10 and 11, inclusive; between 11 and 12 inclusive, or between 12 and 13, inclusive. In certain embodiments, an acidic linker will contain a sequence that is selected from the group consisting of [Gly-Glu]ₙ (SEQ ID NO: 16); [Gly-Gly-Glu]ₙ (SEQ ID NO: 17); [Gly-Gly-Gly-Glu]ₙ (SEQ ID NO: 18); [Gly-Gly-Gly-Gly-Glu]ₙ (SEQ ID NO: 19); [Gly-Asp]n (SEQ ID NO: 20); [Gly-Gly-Asp]ₙ (SEQ ID NO: 21); [Gly-Gly-Gly-Asp], (SEQ ID NO: 22); [Gly-Gly-Gly-Gly-Asp], (SEQ ID NO: 23); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75. In certain embodiments, a basic linker will contain a sequence that is selected from the group consisting of [Gly-Lys]ₙ (SEQ ID NO: 25); [Gly-Gly-Lys]ₙ (SEQ ID NO: 26); [Gly-Gly-Gly-Lys]ₙ (SEQ ID NO: 27); [Gly-Gly-Gly-Gly-Lys]ₙ (SEQ ID NO: 28); [Gly-Arg]ₙ (SEQ ID NO: 29); [Gly-Gly-Arg]ₙ (SEQ ID NO: 30); [Gly-Gly-Gly-Arg]ₙ (SEQ ID NO: 31); [Gly-Gly-Gly-Gly-Arg]ₙ (SEQ ID NO: 32); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

In certain embodiments, the inventive multivalent antibody analogs according comprise, for example, a CH2 domain variant and/or a CH3 domain variant, wherein such variants each independently comprise at least one different amino acid substitution such that a heterodimeric domain pair is generated such that heterodimerization of the first and second polypeptides of the inventive multivalent antibody analogs favored over homodimerization.

With regard to a “variant” of a domain or region of a multivalent antibody analog as used herein throughout, such a variant refers a polypeptide sequence that comprises such a domain or region, and that differs from that of a parent polypeptide sequence by virtue of at least one amino acid modification. The parent polypeptide sequence may be a naturally occurring or wild-type (WT) polypeptide sequence, or may be a modified version of a WT sequence. Preferably, the variant has at least one amino acid modification compared to the parent polypeptide, region, or domain, e.g. from about one to about ten amino acid modifications, and preferably from about one to about five amino acid modifications compared to the parent. The variant polypeptide sequence herein will preferably possess at least about 80% homology with a parent sequence, and most preferably at least about 90% homology, more preferably at least about 95% homology.

By “parent polypeptide”, “parent polypeptide sequence”, “parent protein”, “precursor polypeptide”, or “precursor protein” as used herein is meant an unmodified polypeptide or polypeptide sequence that is subsequently modified to generate a variant polypeptide or polypeptide sequence. Said parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it.

By “Fc variant” or “variant Fc” as used herein is meant an Fc sequence that differs from that of a parent Fc sequence by virtue of at least one amino acid modification. An Fc variant may only encompass an Fc region, or may exist in the context of an antibody, Fc fusion, isolated Fc, Fc fragment, or other polypeptide that is substantially encoded by Fc. Fc variant may refer to the Fc polypeptide itself, compositions comprising the Fc variant polypeptide, or the amino acid sequence that encodes it.

By “Fc polypeptide variant” or “variant Fc polypeptide” as used herein is meant an Fc polypeptide that differs from a parent Fc polypeptide by virtue of at least one amino acid modification. By “Fc variant antibody” or “antibody Fc variant” as used herein is meant an antibody that differs from a parent antibody by virtue of at least one amino acid modification in the Fc region.

By “protein variant” or “variant protein” as used herein is meant a protein that differs from a parent protein by virtue of at least one amino acid modification. By “antibody variant” or “variant antibody” as used herein is meant an antibody that differs from a parent antibody by virtue of at least one amino acid modification. By “IgG variant” or “variant IgG” as used herein is meant an antibody that differs from a parent IgG by virtue of at least one amino acid modification. By “immunoglobulin variant” or “variant immunoglobulin” as used herein is meant an immunoglobulin sequence that differs from that of a parent immunoglobulin sequence by virtue of at least one amino acid modification.

Interaction between heterodimeric pairs or disclosed multivalent antibody analogs comprising such heterodimeric pairs may be promoted at the heterodimeric pair interface by the formation of protuberance-into-cavity complementary regions at such interfaces; the formation of non-naturally occurring disulfide bonds at such interfaces; leucine zipper at such interfaces; hydrophobic regions at such interfaces; and/or hydrophilic regions at such interfaces. “Protuberances” are constructed by replacing small amino acid side chains from the interface of the first polypeptide with larger side chains (e.g. tyrosine or tryptophan). Compensatory “cavities” of identical or similar size to the protuberances are optionally created on the interface of the second polypeptide by replacing large amino acid side chains with smaller ones (e.g. alanine or threonine). Where a suitably positioned and dimensioned protuberance or cavity exists at the interface of either the first or second polypeptide, it is only necessary to engineer a corresponding cavity or protuberance, respectively, at the adjacent interface. Non-naturally occurring disulfide bonds are constructed by replacing on the first polypeptide a naturally occurring amino acid with a free thiol-containing residue, such as cysteine, such that the free thiol interacts with another free thiol-containing residue on the second polypeptide such that a disulfide bond is formed between the first and second polypeptides Exemplary heterodimerization pairs and methods for making such in accordance with the present invention are available in the art, and are disclosed, for example, in US 2011/0054151; US 2007/0098712; and the like.

In certain embodiments, the heterodimeric pairs are contained within the Fc region of the inventive multivalent antibody analogs. Fc regions that contain such heterodimeric pairs are referred to as “heterodimeric Fc regions”

Accordingly, in certain embodiments, multivalent antibody analogs comprise a CH2 and/or a CH3 domain variant, wherein either: a) the CH2 domain variant and the CH3 domain variant each independently comprises a at least one protuberance in either the CH2 domain or the CH3 domain of the first polypeptide and at least one corresponding cavity in the CH2 domain or the CH3 domain of the second; or the CH2 domain variant and the CH3 domain variant each independently comprises at least one cavity in either the CH2 domain or the CH3 domain of the first polypeptide and at least one corresponding protuberance in the CH2 domain or the CH3 domain of the second polypeptide. In certain other embodiments, the multivalent antibody analogs comprise a CH2 and/or a CH3 domain variant, wherein either: a) the CH2 domain variant and the CH3 domain variant each independently comprises at least one substituted negatively-charged amino acid in either the CH2 domain or the CH3 domain of the first polypeptide and at least one corresponding positively-charged amino acid in either the CH2 domain or the CH3 domain of the second polypeptide; or b) the CH2 domain variant and the CH3 domain variant each independently comprises at least one substituted positively-charged amino acid in either the CH2 domain or the CH3 domain of the first polypeptide and at least one corresponding substituted negatively-charged substituted amino acid in either the CH2 domain or the CH3 domain of the second polypeptide.

With regard to Fc function in “natural” antibodies (i.e., those antibodies generated in vivo via native biological antibody synthesis by native B-cells), the Fc region of an antibody interacts with a number of Fc receptors and ligands, imparting an array of important functional capabilities referred to as effector functions. For IgG the Fc region, Fc comprises Ig domains Cγ2 and Cγ3 and the N-terminal hinge leading into Cγ2. An important family of Fc receptors for the IgG class is the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and γδ T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP).

The different IgG subclasses have different affinities for the FcγRs, with IgG1 and IgG3 typically binding substantially better to the receptors than IgG2 and IgG4 (Jefferis et al., 2002, Immunol Lett 82:57-65). The FcγRs bind the IgG Fc region with different affinities. The extracellular domains of FcγRIIIa and FcγRIIIb are 96% identical; however FcγRIIIb does not have a intracellular signaling domain. Furthermore, whereas FcγRI, FcγRIIa/c, and FcγRIIIa are positive regulators of immune complex-triggered activation, characterized by having an intracellular domain that has an immunoreceptor tyrosine-based activation motif (ITAM), FcγRIIb has an immunoreceptor tyrosine-based inhibition motif (ITIM) and is therefore inhibitory. Thus the former are referred to as activation receptors, and FcγRIIb is referred to as an inhibitory receptor. Despite these differences in affinities and activities, all FcγRs bind the same region on Fc, at the N-terminal end of the Cγ2 domain and the preceding hinge.

An overlapping but separate site on Fc serves as the interface for the complement protein C1q. In the same way that Fc/FcγR binding mediates ADCC, Fc/C1q binding mediates complement dependent cytotoxicity (CDC). A site on Fc between the Cγ2 and Cγ3 domains mediates interaction with the neonatal receptor FcRn, the binding of which recycles endocytosed antibody from the endosome back to the bloodstream (Raghavan et al., 1996, Annu Rev Cell Dev Biol 12:181-220; Ghetie et al., 2000, Annu Rev Immunol 18:739-76). This process, coupled with preclusion of kidney filtration due to the large size of the full length molecule, results in favorable antibody serum half-lives ranging from one to three weeks. Binding of Fc to FcRn also plays a key role in antibody transport. The binding site for FcRn on Fc is also the site at which the bacterial proteins A and G bind. The tight binding by these proteins is typically exploited as a means to purify antibodies by employing protein A or protein G affinity chromatography during protein purification. The fidelity of these regions, the complement and FcRn/protein A binding regions are important for both the clinical properties of antibodies and their development.

A particular feature of the Fc region of “natural” antibodies is the conserved N-linked glycosylation that occurs at N297. This carbohydrate, or oligosaccharide as it is sometimes referred, plays a critical structural and functional role for the antibody, and is one of the principle reasons that antibodies must be produced using mammalian expression systems. Efficient Fc binding to FcγR and C1q requires this modification, and alterations in the composition of the N297 carbohydrate or its elimination affect binding to these proteins.

In some embodiments, the inventive multivalent antibody analogs disclosed herein comprise an Fc variant. An Fc variant comprises one or more amino acid modifications relative to a parent Fc polypeptide, wherein the amino acid modification(s) provide one or more optimized properties. Fc variants further comprise either a CH2 domain variant, a CH3 domain variant, or both a CH2 domain variant and a CH3 domain variant. By “modification” herein is meant an alteration in the physical, chemical, or sequence properties of a protein, polypeptide, antibody, inventive multivalent antibody analog, or immunoglobulin. An amino acid modification can be an amino acid substitution, insertion, and/or deletion in a polypeptide sequence. By “amino acid substitution” or “substitution” herein is meant the replacement of an amino acid at a particular position in a parent polypeptide sequence with another amino acid. For example, the substitution Y349T refers to a variant polypeptide, in this case a constant heavy chain variant, in which the tyrosine at position 349 is replaced with threonine. By “amino acid insertion” or “insertion” as used herein is meant the addition of an amino acid at a particular position in a parent polypeptide sequence. By “amino acid deletion” or “deletion” as used herein is meant the removal of an amino acid at a particular position in a parent polypeptide sequence.

An Fc variant disclosed herein differs in amino acid sequence from its parent by virtue of at least one amino acid modification. The inventive multivalent antibody analogs disclosed herein may have more than one amino acid modification as compared to the parent, for example from about one to fifty amino acid modifications, e.g., from about one to ten amino acid modifications, from about one to about five amino acid modifications, etc. compared to the parent. Thus the sequences of the Fc variants and those of the parent Fc polypeptide are substantially homologous. For example, the variant Fc variant sequences herein will possess about 80% homology with the parent Fc variant sequence, e.g., at least about 90% homology, at least about 95% homology, at least about 98% homology, at least about 99% homology, etc. Modifications disclosed herein also include glycoform modifications. Modifications may be made genetically using molecular biology, or may be made enzymatically or chemically.

Fc variants disclosed herein are defined according to the amino acid modifications that compose them. Thus, for example, the substitution Y349T refers to a variant polypeptide, in this case a constant heavy chain variant, in which the tyrosine at position 349 is replaced with threonine. Likewise, Y349T/T394F defines an Fc variant with the substitutions Y349T and T394F relative to the parent Fc polypeptide. The identity of the WT amino acid may be unspecified, in which case the aforementioned variant is referred to as 349T/394F. It is noted that the order in which substitutions are provided is arbitrary, that is to say that, for example, 349T/394F is the same Fc variant as 394F/349T. Unless otherwise noted, constant region and Fc positions discussed herein are numbered according to the EU index or EU numbering scheme (Kabat et al., 1991, Sequences of Proteins of Immunological Interest, 5th Ed., United States Public Health Service, National Institutes of Health, Bethesda). The EU index or EU index as in Kabat or EU numbering scheme refers to the numbering of the EU antibody (Edelman et al., 1969, Proc Natl Acad Sci USA 63:78-85).

In certain embodiments, the Fc variants disclosed herein are based on human IgG sequences, and thus human IgG sequences are used as the “base” sequences against which other sequences are compared, including but not limited to sequences from other organisms, for example rodent and primate sequences. Immunoglobulins may also comprise sequences from other immunoglobulin classes such as IgA, IgE, IgGD, IgGM, and the like. It is contemplated that, although the Fc variants disclosed herein are engineered in the context of one parent IgG, the variants may be engineered in or “transferred” to the context of another, second parent IgG. This is done by determining the “equivalent” or “corresponding” residues and substitutions between the first and second IgG, typically based on sequence or structural homology between the sequences of the first and second IgGs. In order to establish homology, the amino acid sequence of a first IgG outlined herein is directly compared to the sequence of a second IgG. After aligning the sequences, using one or more of the homology alignment programs known in the art (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first immunoglobulin are defined. Alignment of conserved residues may conserve 100% of such residues. However, alignment of greater than 75% or as little as 50% of conserved residues is also adequate to define equivalent residues. Equivalent residues may also be defined by determining structural homology between a first and second IgG that is at the level of tertiary structure for IgGs whose structures have been determined. In this case, equivalent residues are defined as those for which the atomic coordinates of two or more of the main chain atoms of a particular amino acid residue of the parent or precursor (N on N, CA on CA, C on C and O on O) are within about 0.13 nm, after alignment. In another embodiment, equivalent residues are within about 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent IgG in which the IgGs are made, what is meant to be conveyed is that the Fc variants discovered as disclosed herein may be engineered into any second parent IgG that has significant sequence or structural homology with the Fc variant. Thus for example, if a variant antibody is generated wherein the parent antibody is human IgG1, by using the methods described above or other methods for determining equivalent residues, the variant antibody may be engineered in another IgG1 parent antibody that binds a different antigen, a human IgG2 parent antibody, a human IgA parent antibody, a mouse IgG2a or IgG2b parent antibody, and the like. Again, as described above, the context of the parent Fc variant does not affect the ability to transfer the Fc variants disclosed herein to other parent IgGs.

Fc variants that comprise or are CH3 domain variants as described above may comprise at least one substitution at a position in a CH3 domain selected from the group consisting of 349, 351, 354, 356, 357, 364, 366, 368, 370, 392, 394, 395, 396, 397, 399, 401, 405, 407, 409, 411, and 439, wherein numbering is according to the EU index as in Kabat. In a preferred embodiment, CH3 domain variants comprise at least one CH3 domain substitution per heavy chain selected from the group consisting of 349A, 349C, 349E, 349I, 349K, 349S, 349T, 349W, 351 E, 351K, 354C, 356K, 357K, 364C, 364D, 364E, 364F, 364G, 364H, 364R, 364T, 364Y, 366D, 366K, 366S, 366W, 366Y, 368A, 368E, 368K, 368S, 370C, 370D, 370E, 370G, 370R, 370S, 370V, 392D, 392E, 394F, 394S, 394W, 394Y, 395T, 395V, 396T, 397E, 397S, 397T, 399K, 401 K, 405A, 405S, 407T, 407V, 409D, 409E, 411 D, 411 E, 411K, and 439D. Each of these variants can be used individually or in any combination for each heavy chain Fc region. As will be appreciated by those in the art, each heavy chain can comprise different numbers of substitutions. For example, both heavy chains that make up the Fc region may comprise a single substitution, one chain may comprise a single substitution and the other two substitutions, both can contain two substitutions (although each chain will contain different substitutions), etc.

In some embodiments, the CH2 and/or CH3 domain variants are made in combinations, that is, two or more variants per heavy chain Fc domain, selected from the group outlined above.

Other CH2 and/or CH3 domain variants that favor heterodimerization that may be employed in the design and preparation of the inventive multivalent antibody analogs of the invention are provided in, for example, Ridgeway et al., 1996, Protein Engineering 9[7]:617-621; U.S. Pat. No. 5,731,168; Xie et al., 2005, J Immunol Methods 296:95-101; Davis et al., 2010, Protein Engineering, Design & Selection 23[4]:195-202; Gunasekaran et al., 2010, J Biol Chem 285[25]:1937-19646; and PCT/US2009/000071).

The Fc variants disclosed herein may be optimized for improved or reduced binding to Fc receptors or Fc ligands. By “Fc receptor” or “Fc ligand” as used herein is meant a molecule, preferably a polypeptide, from any organism that binds to the Fc region of an antibody to form an Fc-ligand complex. Fc ligands include but are not limited to FcγRs, (as described above, including but not limited to FcγRIIIa, FcγRIIa, FcγRIIb, FcγRI and FcRn), C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs. Fc ligands may include undiscovered molecules that bind Fc.

The inventive multivalent antibody analogs may be designed to optimize properties, including but are not limited to enhanced or reduced affinity for an Fc receptor. By “greater affinity” or “improved affinity” or “enhanced affinity” or “better affinity” than a parent Fc polypeptide, as used herein, is meant that an Fc variant binds to an Fc receptor with a significantly higher equilibrium constant of association (KA or Kₐ) or lower equilibrium constant of dissociation (KD or K_{d}) than the parent Fc polypeptide when the amounts of variant and parent polypeptide in the binding assay are essentially the same. For example, the Fc variant with improved Fc receptor binding affinity may display from about 5 fold to about 1000 fold, e.g. from about 10 fold to about 500 fold improvement in Fc receptor binding affinity compared to the parent Fc polypeptide, where Fc receptor binding affinity is determined, for example, by the binding methods disclosed herein, including but not limited to Biacore, by one skilled in the art. Accordingly, by “reduced affinity” as compared to a parent Fc polypeptide as used herein is meant that an Fc variant binds an Fc receptor with significantly lower KA or higher KD than the parent Fc polypeptide. Greater or reduced affinity can also be defined relative to an absolute level of affinity.

In one embodiment, particularly useful Fc modifications for the present invention are variants that reduce or ablate binding to one or more FcγRs and/or complement proteins, thereby reducing or ablating Fc-mediated effector functions such as ADCC, ADCP, and CDC. Such variants are also referred to herein as “knockout variants” or “KO variants”. Variants that reduce binding to FcγRs and complement are useful for reducing unwanted interactions mediated by the Fc region and for tuning the selectivity of the inventive multivalent antibody analogs. Preferred knockout variants are described in U.S. Ser. No. 11/981,606, filed Oct. 31, 2007, entitled “Fc Variants with Optimized Properties”. Preferred modifications include but are not limited substitutions, insertions, and deletions at positions 234, 235, 236, 237, 267, 269, 325, and 328, wherein numbering is according to the EU index. Preferred substitutions include but are not limited to 234G, 235G, 236R, 237K, 267R, 269R, 325L, and 328R, wherein numbering is according to the EU index. A preferred variant comprises 236R/328R. Variants may be used in the context of any IgG isotype or IgG isotype Fc region, including but not limited to human IgG1, IgG2, IgG3, and/or IgG4 and combinations thereof. Preferred IgG Fc regions for reducing FcγR and complement binding and reducing Fc-mediated effector functions are IgG2 and IgG4 Fc regions. Hybrid isotypes may also be useful, for example hybrid IgG1/IgG2 isotypes as described in US 2006-0134105. Other modifications for reducing FcγR and complement interactions include but are not limited to substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Fc modifications that improve binding to FcγRs and/or complement are also amenable to incorporation in the design and preparation of the inventive multivalent antibody analogs disclosed herein. Such Fc variants may enhance Fc-mediated effector functions such as ADCC, ADCP, and/or CDC. Preferred modifications for improving FcγR and complement binding are described in, e.g., U.S. Pat. No. 8,188,231 and US 2006-0235208. Preferred modifications comprise a substitution at a position selected from the group consisting of 236, 239, 268, 324, and 332, wherein numbering is according to the EU index. Preferred substitutions include but are not limited to 236A, 239D, 239E, 268D, 267E, 268E, 268F, 324T, 332D, and 332E. Preferred variants include but are not limited to 239D/332E, 236A/332E, 236A/239D/332E, 268F/324T, 267E/268F, 267E/324T, and 267E/268F/324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl, 2009, ibid.

In one embodiment, the inventive multivalent antibody analogs disclosed herein may incorporate Fc variants that enhance affinity for an inhibitory receptor FcγRIIb. Such variants may provide the inventive multivalent antibody analogs herein with immunomodulatory activities related to FcγRIIb⁺ cells, including for example B cells and monocytes. In one embodiment, the Fc variants provide selectively enhanced affinity to FcγRIIb relative to one or more activating receptors. Modifications for altering binding to FcγRIIb are described in U.S. Pat. No. 8,063,187, filed May 30, 2008, entitled “Methods and Compositions for Inhibiting CD32b Expressing Cells”. In particular, Fc variants that improve binding to FcγRIIb may include one or more modifications at a position selected from the group consisting of 234, 235, 236, 237, 239, 266, 267, 268, 325, 326, 327, 328, and 332, according to the EU index. Preferable substitutions for enhancing FcγRIIb affinity include but are not limited to 234D, 234E, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. More preferably, substitutions include but are not limited to 235Y, 236D, 239D, 266M, 267E, 268D, 268E, 328F, 328W, and 328Y. Preferred Fc variants for enhancing binding to FcγRIIb include but are not limited to 235Y/267E, 236D/267E, 239D/268D, 239D/267E, 267E/268D, 267E/268E, and 267E/328F.

In some embodiments, the inventive multivalent antibody analogs disclosed herein may incorporate Fc variants that improve FcRn binding. Such variants may enhance the in vivo pharmacokinetic properties of the inventive multivalent antibody analogs. Preferred variants that increase binding to FcRn and/or improve pharmacokinetic properties include but are not limited to substitutions at positions 259, 308, 428, and 434, including but not limited to for example 2591, 308F, 428L, 428M, 434S, 434H, 434F, 434Y, 434M, 428L/4345, 2591/308F and 2591/308F/428L (and others described in U.S. Ser. No. 12/341,769, filed Dec. 22, 2008, entitled “Fc Variants with Altered Binding to FcRn”). Other variants that increase Fc binding to FcRn include but are not limited to: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 311S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671.

The inventive multivalent antibody analogs disclosed herein can incorporate F_{c} modifications in the context of any IgG isotype or IgG isotype Fc region, including but not limited to human IgG1, IgG2, IgG3, and/or IgG4. The IgG isotype may be selected such as to alter FcγR- and/or complement-mediated effector function(s). Hybrid IgG isotypes may also be useful. For example, US 2006-0134105describes a number of hybrid IgG1/IgG2 constant regions that may find use in the particular invention. In some embodiments of the invention, inventive multivalent antibody analogs may comprise means for isotypic modifications, that is, modifications in a parent IgG to the amino acid type in an alternate IgG. For example, an IgG1/IgG3 hybrid variant may be constructed by a substitutional means for substituting IgG1 positions in the CH2 and/or CH3 region with the amino acids from IgG3 at positions where the two isotypes differ. Thus a hybrid variant IgG antibody may be constructed that comprises one or more substitutional means, e.g., 274Q, 276K, 300F, 339T, 356E, 358M, 384S, 392N, 397M, 4221, 435R, and 436F. In other embodiments of the invention, an IgG1/IgG2 hybrid variant may be constructed by a substitutional means for substituting IgG2 positions in the CH2 and/or CH3 region with amino acids from IgG1 at positions where the two isotypes differ. Thus a hybrid variant IgG antibody may be constructed that comprises one or more substitutional means, e.g., one or more of the following amino acid substations: 233E, 234L, 235L, −236G (referring to an insertion of a glycine at position 236), and 327A.

All antibodies contain carbohydrate at conserved positions in the constant regions of the heavy chain. Each antibody isotype has a distinct variety of N-linked carbohydrate structures. Aside from the carbohydrate attached to the heavy chain, up to 30% of human IgGs have a glycosylated Fab region. IgG has a single N-linked biantennary carbohydrate at Asn297 of the CH2 domain. For IgG from either serum or produced ex vivo in hybridomas or engineered cells, the IgG are heterogeneous with respect to the Asn297 linked carbohydrate. For human IgG, the core oligosaccharide normally consists of GlcNAc2Man3GlcNAc, with differing numbers of outer residues.

The inventive multivalent antibody analogs herein may also comprise carbohydrate moieties, which moieties will be described with reference to commonly used nomenclature for the description of oligosaccharides. A review of carbohydrate chemistry which uses this nomenclature is found in Hubbard et al. 1981, Ann. Rev. Biochem. 50:555-583. This nomenclature includes, for instance, Man, which represents mannose; GlcNAc, which represents 2-N-acetylglucosamine; Gal which represents galactose; Fuc for fucose; and Glc, which represents glucose. Sialic acids are described by the shorthand notation NeuNAc, for 5-N-acetylneuraminic acid, and NeuNGc for 5-glycolylneuraminic.

The term “glycosylation” means the attachment of oligosaccharides (carbohydrates containing two or more simple sugars linked together e.g. from two to about twelve simple sugars linked together) to a glycoprotein. The oligosaccharide side chains are typically linked to the backbone of the glycoprotein through either N- or O-linkages. The oligosaccharides of inventive multivalent antibody analogs disclosed herein occur generally are attached to a CH2 domain of an Fc region as N-linked oligosaccharides. “N-linked glycosylation” refers to the attachment of the carbohydrate moiety to an asparagine residue in a glycoprotein chain. The skilled artisan will recognize that, for example, each of murine IgG1, IgG2a, IgG2b and IgG3 as well as human IgG1, IgG2, IgG3, IgG4, IgA and IgD CH2 domains have a single site for N-linked glycosylation at residue 297.

For the purposes herein, a “mature core carbohydrate structure” refers to a processed core carbohydrate structure attached to an Fc region which generally consists of the following carbohydrate structure GlcNAc(Fucose)-GlcNAc-Man-(Man-GlcNAc)₂typical of biantennary oligosaccharides. The mature core carbohydrate structure is attached to the Fc region of the glycoprotein, generally via N-linkage to Asn297 of a CH2 domain of the Fc region. A “bisecting GlcNAc” is a GlcNAc residue attached to the α1,4 mannose of the mature core carbohydrate structure. The bisecting GlcNAc can be enzymatically attached to the mature core carbohydrate structure by a α(1,4)-N-acetylglucosaminyltransferase III enzyme (GnTIII). CHO cells do not normally express GnTIII (Stanley et al., 1984, J. Biol. Chem. 261:13370-13378), but may be engineered to do so (Umana et al., 1999, Nature Biotech. 17:176-180).

Described herein are multivalent antibody analogs that comprise modified glycoforms or engineered glycoforms. By “modified glycoform” or “engineered glycoform” as used herein is meant a carbohydrate composition that is covalently attached to a protein, for example an antibody, wherein said carbohydrate composition differs chemically from that of a parent protein. Engineered glycoforms may be useful for a variety of purposes, including but not limited to enhancing or reducing FcγR-mediated effector function. In one embodiment, the inventive multivalent antibody analogs disclosed herein are modified to control the level of fucosylated and/or bisecting oligosaccharides that are covalently attached to the Fc region.

A variety of methods are well known in the art for generating modified glycoforms (Umana et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473; U.S. Ser. No. 12/434,533). These techniques control the level of fucosylated and/or bisecting oligosaccharides that are covalently attached to the Fc region, for example by expressing an IgG in various organisms or cell lines, engineered or otherwise (for example Lec-13 CHO cells or rat hybridoma YB2/0 cells), by regulating enzymes involved in the glycosylation pathway (for example FUT8 [α-1,6-fucosyltranserase] and/or β1-4-N-acetylglucosaminyltransferase III [GnTIII]), by modifying carbohydrate(s) after the IgG has been expressed, or by expressing antibody in the presence of fucose analogs as enzymatic inhibitors. Other methods for modifying glycoforms of the inventive multivalent antibody analogs disclosed herein include using glycoengineered strains of yeast (Li et al., 2006, Nature Biotechnology 24(2):210-215), moss (Nechansky et al., 2007, Mol Immunjol 44(7):1826-8), and plants (Cox et al., 2006, Nat Biotechnol 24(12):1591-7). The use of a particular method to generate a modified glycoform is not meant to constrain embodiments to that method. Rather, embodiments disclosed herein encompass inventive multivalent antibody analogs with modified glycoforms irrespective of how they are produced.

In one embodiment, the inventive multivalent antibody analogs disclosed herein are glycoengineered to alter the level of sialylation. Higher levels of sialylated Fc glycans in immunoglobulin G molecules can adversely impact functionality (Scallon et al., 2007, Mol. Immunol. 44(7):1524-34), and differences in levels of Fc sialylation can result in modified anti-inflammatory activity (Kaneko et al., 2006, Science 313:670-673). Because antibodies may acquire anti-inflammatory properties upon sialylation of Fc core polysaccharide, it may be advantageous to glycoengineer the inventive multivalent antibody analogs disclosed herein for greater or reduced Fc sialic acid content.

“Engineered glycoform” typically refers to the different carbohydrate or oligosaccharide; thus for example an immuoglobulin may comprise an engineered glycoform. In one embodiment, a composition disclosed herein comprises a glycosylated inventive multivalent antibody analog having an Fc region, wherein about 51-100% of the glycosylated antibody, e.g., 80-100%, 90-100%, 95-100%, etc. of the antibody in the composition comprises a mature core carbohydrate structure which lacks fucose. In another embodiment, the antibody in the composition both comprises a mature core carbohydrate structure that lacks fucose and additionally comprises at least one amino acid modification in the Fc region. In an alternative embodiment, a composition comprises a glycosylated inventive multivalent antibody analog having an Fc region, wherein about 51-100% of the glycosylated antibody, 80-100%, or 90-100%, of the antibody in the composition comprises a mature core carbohydrate structure which lacks sialic acid. In another embodiment, the antibody in the composition both comprises a mature core carbohydrate structure that lacks sialic acid and additionally comprises at least one amino acid modification in the Fc region. In yet another embodiment, a composition comprises a glycosylated inventive multivalent antibody analog having an Fc region, wherein about 51-100% of the glycosylated antibody, 80-100%, or 90-100%, of the antibody in the composition comprises a mature core carbohydrate structure which contains sialic acid. In another embodiment, the antibody in the composition both comprises a mature core carbohydrate structure that contains sialic acid and additionally comprises at least one amino acid modification in the Fc region. In another embodiment, the combination of engineered glycoform and amino acid modification provides optimal Fc receptor binding properties to the antibody.

The inventive multivalent antibody analogs disclosed herein may comprise one or more modifications that provide additional optimized properties. Said modifications may be amino acid modifications, or may be modifications that are made enzymatically or chemically. Such modification(s) likely provide some improvement in the inventive multivalent antibody analog, for example an enhancement in its stability, solubility, function, or clinical use. Disclosed herein are a variety of improvements that may be made by coupling the inventive multivalent antibody analogs disclosed herein with additional modifications.

In one embodiment, at least one variable region of multivalent antibody analog disclosed herein may be affinity matured, that is to say that amino acid modifications have been made in the VH and/or VL domains to enhance binding of the antibody to its target antigen. Such types of modifications may improve the association and/or the dissociation kinetics for binding to the target antigen. Other modifications include those that improve selectivity for target antigen vs. alternative targets. These include modifications that improve selectivity for antigen expressed on target vs. non-target cells. Inventive multivalent antibody analogs disclosed herein may comprise one or more modifications that provide reduced or enhanced internalization of an inventive multivalent antibody analog.

In other embodiments embodiment, modifications are made to improve biophysical properties of the inventive multivalent antibody analogs disclosed herein, including but not limited to stability, solubility, and oligomeric state. Modifications can include, for example, substitutions that provide more favorable intramolecular interactions in the inventive multivalent antibody analog such as to provide greater stability, or substitution of exposed nonpolar amino acids with polar amino acids for higher solubility. Other modifications to the inventive multivalent antibody analogs disclosed herein include those that enable the specific formation or homodimeric or homomultimeric molecules. Such modifications include but are not limited to engineered disulfides, as well as chemical modifications or aggregation methods.

In further embodiments, the inventive multivalent antibody analogs disclosed herein comprise modifications that remove proteolytic degradation sites. These may include, for example, protease sites that reduce production yields, as well as protease sites that degrade the administered protein in vivo. In one embodiment, additional modifications are made to remove covalent degradation sites such as deamidation (i.e. deamidation of glutaminyl and asparaginyl residues to the corresponding glutamyl and aspartyl residues), oxidation, and proteolytic degradation sites. Deamidation sites that are particular useful to remove are those that have enhance propensity for deamidation, including, but not limited to asparaginyl and gltuamyl residues followed by glycines (NG and QG motifs, respectively). In such cases, substitution of either residue can significantly reduce the tendency for deamidation. Common oxidation sites include methionine and cysteine residues. Other covalent modifications, that can either be introduced or removed, include hydroxylation of proline and lysine, phosphorylation of hydroxyl groups of seryl or threonyl residues, methylation of the “-amino groups of lysine, arginine, and histidine side chains, acetylation of the N-terminal amine, and amidation of any C-terminal carboxyl group. Additional modifications also may include but are not limited to posttranslational modifications such as N-linked or O-linked glycosylation and phosphorylation.

Modifications may include those that improve expression and/or purification yields from hosts or host cells commonly used for production of biologics. These include, but are not limited to various mammalian cell lines (e.g. CHO, HEK, COS, NIH LT3, Saos, and the like), yeast cells, bacterial cells, and plant cells. Additional modifications include modifications that remove or reduce the ability of heavy chains to form inter-chain disulfide linkages. Additional modifications include modifications that remove or reduce the ability of heavy chains to form intra-chain disulfide linkages.

The inventive multivalent antibody analogs disclosed herein may comprise modifications that include the use of unnatural amino acids incorporated using, including but not limited to methods described in Liu & Schultz, 2010, Annu Rev Biochem 79:413-444. In some embodiments, these modifications enable manipulation of various functional, biophysical, immunological, or manufacturing properties discussed above. In additional embodiments, these modifications enable additional chemical modification for other purposes.

Other modifications are contemplated herein. For example, the inventive multivalent antibody analogs may be linked to one of a variety of nonproteinaceous polymers, e.g., polyethylene glycol (PEG), polypropylene glycol, polyoxyalkylenes, or copolymers of polyethylene glycol and polypropylene glycol. Additional amino acid modifications may be made to enable specific or non-specific chemical or posttranslational modification of the inventive multivalent antibody analogs. Such modifications, include, but are not limited to PEGylation and glycosylation. Specific substitutions that can be utilized to enable PEGylation include, but are not limited to, introduction of novel cysteine residues or unnatural amino acids such that efficient and specific coupling chemistries can be used to attach a PEG or otherwise polymeric moiety. Introduction of specific glycosylation sites can be achieved by introducing novel N-X-T/S sequences into the inventive multivalent antibody analogs disclosed herein.

Modifications to reduce immunogenicity may include modifications that reduce binding of processed peptides derived from the parent sequence to MHC proteins. For example, amino acid modifications would be engineered such that there are no or a minimal number of immune epitopes that are predicted to bind, with high affinity, to any prevalent MHC alleles. Several methods of identifying MHC-binding epitopes in protein sequences are known in the art and may be used to score epitopes in an antibody disclosed herein.

Covalent modifications are included within the scope of inventive multivalent antibody analogs disclosed herein, and are generally, but not always, done post-translationally. For example, several types of covalent modifications can be introduced into the molecule by reacting specific amino acid residues with an organic derivatizing agent that is capable of reacting with selected side chains or the N- or C-terminal residues. In some embodiments, the covalent modification of the inventive multivalent antibody analogs disclosed herein comprises the addition of one or more labels. The term “labeling group” means any detectable label. In some embodiments, the labeling group is coupled to the inventive multivalent antibody analog via spacer arms of various lengths to reduce potential steric hindrance. Various methods for labeling proteins are known in the art and may be used in generating inventive multivalent antibody analogs disclosed herein.

In certain embodiments, the inventive multivalent antibody analogs disclosed herein comprise “fusion proteins”, also referred to herein as “conjugates”. The fusion partner or conjugate partner can be proteinaceous or non-proteinaceous; the latter generally being generated using functional groups on the inventive multivalent antibody analog and on the conjugate partner. Conjugate and fusion partners may be any molecule, including small molecule chemical compounds and polypeptides. For example, a variety of conjugates and methods are described in Trail et al., 1999, Curr. Opin. Immunol. 11:584-588. Possible conjugate partners include but are not limited to cytokines, cytotoxic agents, toxins, radioisotopes, chemotherapeutic agent, anti-angiogenic agents, a tyrosine kinase inhibitors, and other therapeutically active agents. In some embodiments, conjugate partners may be thought of more as payloads, that is to say that the goal of a conjugate is targeted delivery of the conjugate partner to a targeted cell, for example a cancer cell or immune cell, by the multivalent antibody analogs. Thus, for example, the conjugation of a toxin to an multivalent antibody analogs targets the delivery of said toxin to cells expressing the target antigen. As will be appreciated by one skilled in the art, in reality the concepts and definitions of fusion and conjugate are overlapping. The designation of a fusion or conjugate is not meant to constrain it to any particular embodiment disclosed herein. Rather, these terms are used to convey the broad concept that any multivalent antibody analogs disclosed herein may be linked genetically, chemically, or otherwise, to one or more polypeptides or molecules to provide some desirable property.

Suitable conjugates include, but are not limited to, labels as described below, drugs and cytotoxic agents including, but not limited to, cytotoxic drugs (e.g., chemotherapeutic agents) or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to inventive multivalent antibody analog, or binding of a radionuclide to a chelating agent that has been covalently attached to the inventive multivalent antibody analog. Additional embodiments utilize calicheamicin, auristatins, geldanamycin, maytansine, and duocarmycins and analogs. Antibody-drug conjugates are described in Alley et al., 2010, Curr Opin Chem Biol 14[4]:529-37.

In certain embodiments, the inventive multivalent antibody analogs disclosed herein are fused or conjugated to a cytokine By “cytokine” as used herein is meant a generic term for proteins released by one cell population that act on another cell as intercellular mediators. For example, as described in Penichet et al., 2001, J. Immunol. Methods 248:91-101, cytokines may be fused to an inventive multivalent antibody analog to provide an array of desirable properties. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; C5a; and other polypeptide factors including LIF and kit ligand (KL). As used herein, the term cytokine includes proteins from natural sources or from recombinant cell culture, and biologically active equivalents of the native sequence cytokines.

In further embodiments, the inventive multivalent antibody analogs disclosed herein may be conjugated to a “receptor” (such streptavidin) for utilization in tumor pretargeting wherein the analog-receptor conjugate is administered to the patient, followed by removal of unbound conjugate from the circulation using a clearing agent and then administration of a “ligand” (e.g. avidin) which is conjugated to a cytotoxic agent (e.g. a radionucleotide). In an alternate embodiment, the inventive multivalent antibody analog is conjugated or operably linked to an enzyme in order to employ Antibody Dependent Enzyme Mediated Prodrug Therapy (ADEPT). ADEPT may be used by conjugating or operably linking the inventive multivalent antibody analog to a prodrug-activating enzyme that converts a prodrug (e.g. a peptidyl chemotherapeutic agent.

Also disclosed herein are methods for producing and experimentally testing the inventive multivalent antibody analogs. The disclosed methods are not meant to constrain embodiments to any particular application or theory of operation. Rather, the provided methods are meant to illustrate generally that one or more multivalent antibody analogs of the invention may be produced and experimentally tested to obtain inventive multivalent antibody analogs. General methods for antibody molecular biology, expression, purification, and screening are described in Antibody Engineering, edited by Kontermann & Dubel, Springer, Heidelberg, 2001; and Hayhurst & Georgiou, 2001, Curr Opin Chem Biol 5:683-689; Maynard & Georgiou, 2000, Annu Rev Biomed Eng 2:339-76.

In one embodiment disclosed herein, nucleic acids are created that encode the inventive multivalent antibody analogs, and that may then be cloned into host cells, such as yeast cells or mammalian cells, expressed and assayed, if desired. Thus, nucleic acids, and particularly DNA, may be made that encode each protein sequence. These practices are carried out using well-known procedures. For example, a variety of methods that may find use in generating inventive multivalent antibody analogs disclosed herein are described in Molecular Cloning—A Laboratory Manual, 3rd Ed. (Maniatis, Cold Spring Harbor Laboratory Press, New York, 2001), and Current Protocols in Molecular Biology (John Wiley & Sons). There are a variety of techniques that may be used to efficiently generate DNA encoding inventive multivalent antibody analogs disclosed herein. Such methods include but are not limited to gene assembly methods, PCR-based method and methods which use variations of PCR, ligase chain reaction-based methods, pooled oligo methods such as those used in synthetic shuffling, error-prone amplification methods and methods which use oligos with random mutations, classical site-directed mutagenesis methods, cassette mutagenesis, and other amplification and gene synthesis methods. As is known in the art, there are a variety of commercially available kits and methods for gene assembly, mutagenesis, vector subcloning, and the like, and such commercial products find use in for generating nucleic acids that encode inventive multivalent antibody analogs.

The inventive multivalent antibody analogs disclosed herein may be produced by culturing a host cell transformed with nucleic acid, e.g., expression vectors containing nucleic acid encoding the first and second polypeptides of inventive multivalent antibody analogs, under the appropriate conditions to induce or cause expression of the polypeptides. The conditions appropriate for expression will vary with the choice of the expression vector and the host cell, and will be easily ascertained by one skilled in the art through routine experimentation. A wide variety of appropriate host cells may be used, including but not limited to mammalian cells, bacteria, insect cells, yeast cells, and plant cells. For example, a variety of cell lines that may find use in generating inventive multivalent antibody analogs disclosed herein are described in the ATCC® cell line catalog, available from the American Type Culture Collection.

In certain embodiments, the inventive multivalent antibody analogs are expressed in mammalian expression systems, including systems in which the expression constructs are introduced into the mammalian cells using virus such as retrovirus or adenovirus. Any mammalian cells may be used, e.g., human, mouse, rat, hamster, and primate cells. Suitable cells also include known research cells, including but not limited to Jurkat T cells, NIH3T3, CHO, BHK, COS, HEK293, PER C.6, HeLa, Sp2/0, NS0 cells and variants thereof. In an alternate embodiment, library proteins are expressed in bacterial cells. Bacterial expression systems are well known in the art, and include Escherichia coli (E. coli), Bacillus subtilis, Streptococcus cremoris, and Streptococcus lividans. In alternate embodiments, inventive multivalent antibody analogs are produced in insect cells (e.g. Sf21/Sf9, Trichoplusia ni Bti-Tn5b1-4) or yeast cells (e.g. S. cerevisiae, Pichia, etc). In an alternate embodiment, inventive multivalent antibody analogs are expressed in vitro using cell free translation systems. In vitro translation systems derived from both prokaryotic (e.g. E. coli) and eukaryotic (e.g. wheat germ, rabbit reticulocytes) cells are available and may be chosen based on the expression levels and functional properties of the protein of interest. For example, as appreciated by those skilled in the art, in vitro translation is required for some display technologies, for example ribosome display. In addition, the inventive multivalent antibody analogs may be produced by chemical synthesis methods. Also transgenic expression systems both animal (e.g. cow, sheep or goat milk, embryonated hen's eggs, whole insect larvae, etc.) and plant (e.g. corn, tobacco, duckweed, etc.)

The nucleic acids that encode the first and second polypeptides of inventive multivalent antibody analogs disclosed herein may be incorporated into one or more expression vectors, as appropriate, in order to express the encoded polypeptides. A variety of expression vectors may be utilized for protein expression. Expression vectors may comprise self-replicating extra-chromosomal vectors or vectors which integrate into a host genome. Expression vectors are constructed to be compatible with the host cell type. Thus expression vectors which find use in generating inventive multivalent antibody analogs disclosed herein include but are not limited to those which enable protein expression in mammalian cells, bacteria, insect cells, yeast cells, and in vitro systems. As is known in the art, a variety of expression vectors are available, commercially or otherwise, that may find use for expressing inventive multivalent antibody analogs disclosed herein.

Expression vectors typically comprise a protein or polypeptide to be expressed, which is operably linked with control or regulatory sequences, selectable markers, any fusion partners, and/or additional elements. By “operably linked” herein is meant that the nucleic acid is placed into a functional relationship with another nucleic acid sequence. Generally, these expression vectors include transcriptional and translational regulatory nucleic acid operably linked to the nucleic acid encoding the inventive multivalent antibody analog, and are typically appropriate to the host cell used to express the protein. In general, the transcriptional and translational regulatory sequences may include promoter sequences, ribosomal binding sites, transcriptional start and stop sequences, translational start and stop sequences, and enhancer or activator sequences. As is also known in the art, expression vectors typically contain a selection gene or marker to allow the selection of transformed host cells containing the expression vector. Selection genes are well known in the art and will vary with the host cell used.

The first and second polypeptides of the invention may each be independently operably linked to a fusion partner to enable targeting of the expressed polypeptide and/or multivalent antibody analog, purification, screening, display, and the like. Fusion partners may be linked to the inventive multivalent antibody analog sequence via a linker sequences. The linker sequence will generally comprise a small number of amino acids, typically less than ten, although longer linkers may also be used. Typically, linker sequences are selected to be flexible and resistant to degradation. As will be appreciated by those skilled in the art, any of a wide variety of sequences may be used as linkers. For example, a common linker sequence comprises the amino acid sequence GGGGS (SEQ ID NO: 37). A fusion partner may be a targeting or signal sequence that directs inventive multivalent antibody analog and any associated fusion partners to a desired cellular location or to the extracellular media. As is known in the art, certain signaling sequences may target a protein to be either secreted into the growth media, or into the periplasmic space, located between the inner and outer membrane of the cell. A fusion partner may also be a sequence that encodes a peptide or protein that enables purification and/or screening. Such fusion partners include but are not limited to polyhistidine tags (His-tags) (for example H6 (SEQ ID NO: 38) and H10 (SEQ ID NO: 39) or other tags for use with Immobilized Metal Affinity Chromatography (IMAC) systems (e.g. Ni+2 affinity columns)), GST fusions, MBP fusions, Strep-tag, the BSP biotinylation target sequence of the bacterial enzyme BirA, and epitope tags which are targeted by antibodies (for example c-myc tags, flag-tags, and the like). As will be appreciated by those skilled in the art, such tags may be useful for purification, for screening, or both. For example, an inventive multivalent antibody analog may be purified using a His-tag by immobilizing it to a Ni+2 affinity column, and then after purification the same His-tag may be used to immobilize the antibody to a Ni+2 coated plate to perform an ELISA or other binding assay (as described below). A fusion partner may enable the use of a selection method to screen inventive multivalent antibody analogs (see below). Fusion partners that enable a variety of selection methods are well-known in the art.

For example, by fusing the members of an inventive multivalent antibody analog library to the gene III protein, phage display can be employed. Fusion partners may enable inventive multivalent antibody analogs to be labeled. Alternatively, a fusion partner may bind to a specific sequence on the expression vector, enabling the fusion partner and associated inventive multivalent antibody analog to be linked covalently or noncovalently with the nucleic acid that encodes them. The methods of introducing exogenous nucleic acid into host cells are well known in the art, and will vary with the host cell used. Techniques include but are not limited to dextran-mediated transfection, calcium phosphate precipitation, calcium chloride treatment, polybrene mediated transfection, protoplast fusion, electroporation, viral or phage infection, encapsulation of the polynucleotide(s) in liposomes, and direct microinjection of the DNA into nuclei. In the case of mammalian cells, transfection may be either transient or stable.

In certain embodiments, the multivalent antibody analogs are purified or isolated after expression. The multivalent antibody analogs may be isolated or purified in a variety of ways known to those skilled in the art. Purification may be particularly useful in the invention for separating heterodimeric heavy chain species from homodimeric heavy chain species, as described herein. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, isoelectric focusing, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use for purification of inventive multivalent antibody analogs disclosed herein. For example, the bacterial proteins A and G bind to the Fc region. Likewise, the bacterial protein L binds to the Fab region of some antibodies, as of course does the antibody's target antigen. Purification can often be enabled by a particular fusion partner. For example, inventive multivalent antibody analogs may be purified using glutathione resin if a GST fusion is employed, Ni+2 affinity chromatography if a His-tag is employed, or immobilized anti-flag antibody if a flag-tag is used. For general guidance in suitable purification techniques, see, e.g. Protein Purification: Principles and Practice, 3rd Ed., Scopes, Springer-Verlag, NY, 1994. The degree of purification necessary will vary depending on the screen or use of the inventive multivalent antibody analogs. In some instances no purification is necessary. For example in one embodiment, if the inventive multivalent antibody analogs are secreted, screening may take place directly from the media. As is well known in the art, some methods of selection do not involve purification of proteins.

Virtually any antigen may be targeted by the inventive multivalent antibody analogs disclosed herein, including but not limited to proteins, subunits, domains, motifs, and/or epitopes belonging to the following list of target antigens, which includes both soluble factors such as cytokines and membrane-bound factors, including transmembrane receptors: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Ax1, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CAl25, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-1 (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxinl, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC(HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (FLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular FLAP-like alkaline phosphatase, TfR, TGF, TGF-alpha, TGF-beta, TGF-beta Pan Specific, TGF-beta RI (ALK-5), TGF-beta RII, TGF-beta RII, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1Apo-2, DR4), TNFRSF10B (TRAIL R2DRS, KILLER, TRICK-2A, TRICK-B), TNFRSF10C (TRAIL R3DcR1, LIT, TRID), TNFRSF10D (TRAIL R4DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3M68, TR6), TNFRSF7 (CD27), TNFRSF8 (CD30), TNFRSF9 (4-1BB CD137, ILA), TNFRSF21 (DR6), TNFRSF22 (DcTRAIL R2TNFRH2), TNFRST23 (DcTRAIL R1TNFRH1), TNFRSF25 (DR3Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNTSA, WNTSB, WNT6, WNTSA, WNTSB, WNTSA, WNTSB, WNT9A, WNT9A, WNT9B, WNT10A, WNT10B, WNT11, WNT16, XCL1, XCL2, XCR1, XCR1, XEDAR, XIAP, XPD, and receptors for hormones and growth factors.

Exemplary antigens that may be targeted specifically by the multivalent antibody analogs of the invention include but are not limited to: CD20, CD19, Her2, EGFR, EpCAM, c-MET, CD3, FcγRIIIa (CD16), FcγRIIa (CD32a), FcγRIIb (CD32b), FcγRI (CD64), Toll-like receptors (TLRs) such as TLR4 and TLR9, cytokines such as IL-2, IL-5, IL-13, IL-12, IL-23, and TNFα, cytokine receptors such as IL-2R, chemokines, chemokine receptors, growth factors such as VEGF and HGF, and the like.

The choice of suitable target antigens and co-targets depends on the desired therapeutic application. Some targets that have proven especially amenable to antibody therapy are those with signaling functions. Other therapeutic antibodies exert their effects by blocking signaling of the receptor by inhibiting the binding between a receptor and its cognate ligand. Another mechanism of action of therapeutic antibodies is to cause receptor down regulation. Other antibodies do not work by signaling through their target antigen. The choice of co-targets will depend on the detailed biology underlying the pathology of the indication that is being treated.

Monoclonal antibody therapy has emerged as an important therapeutic modality for cancer (Weiner et al., 2010, Nature Reviews Immunology 10:317-327; Reichert et al., 2005, Nature Biotechnology 23[9]:1073-1078). For anti-cancer treatment it may be desirable to target one antigen (antigen-1) whose expression is restricted to the cancerous cells while co-targeting a second antigen (antigen-2) that mediates some immunological killing activity. For other treatments it may be beneficial to co-target two antigens, for example two angiogenic factors or two growth factors that are each known to play some role in proliferation of the tumor. Exemplary co-targets for oncology include but are not limited to HGF and VEGF, IGF-1R and VEGF, Her2 and VEGF, CD19 and CD3, CD20 and CD3, Her2 and CD3, CD19 and FcγRIIIa, CD20 and FcγRIIIa, Her2 and FcγRIIIa. An inventive multivalent antibody analog of the invention may be capable of binding VEGF and phosphatidylserine; VEGF and ErbB3; VEGF and PLGF; VEGF and ROBO4; VEGF and BSG2; VEGF and CDCP1; VEGF and ANPEP; VEGF and c-MET; HER-2 and ERB3; HER-2 and BSG2; HER-2 and CDCP1; HER-2 and ANPEP; EGFR and CD64; EGFR and BSG2; EGFR and CDCP1; EGFR and ANPEP; IGF1R and PDGFR; IGF1R and VEGF; IGF1R and CD20; CD20 and CD74; CD20 and CD30; CD20 and DR4; CD20 and VEGFR2; CD20 and CD52; CD20 and CD4; HGF and c-MET; HGF and NRP1; HGF and phosphatidylserine; ErbB3 and IGF1R; ErbB3 and IGF1,2; c-Met and Her-2; c-Met and NRP1; c-Met and IGF1R; IGF1,2 and PDGFR; IGF1,2 and CD20; IGF1,2 and IGF1R; IGF2 and EGFR; IGF2 and HER2; IGF2 and CD20; IGF2 and VEGF; IGF2 and IGF1R; IGF1 and IGF2; PDGFRa and VEGFR2; PDGFRa and PLGF; PDGFRa and VEGF; PDGFRa and c-Met; PDGFRa and EGFR; PDGFRb and VEGFR2; PDGFRb and c-Met; PDGFRb and EGFR; RON and c-Met; RON and MTSP1; RON and MSP; RON and CDCP1; VGFR1 and PLGF; VGFR1 and RON; VGFR1 and EGFR; VEGFR2 and PLGF; VEGFR2 and NRP1; VEGFR2 and RON; VEGFR2 and DLL4; VEGFR2 and EGFR; VEGFR2 and ROBO4; VEGFR2 and CD55; LPA and Si P; EPHB2 and RON; CTLA4 and VEGF; CD3 and EPCAM; CD40 and IL6; CD40 and IGF; CD40 and CD56; CD40 and CD70; CD40 and VEGFR1; CD40 and DR5; CD40 and DR4; CD40 and APRIL; CD40 and BCMA; CD40 and RANKL; CD28 and MAPG; CD80 and CD40; CD80 and CD30; CD80 and CD33; CD80 and CD74; CD80 and CD2; CD80 and CD3; CD80 and CD19; CD80 and CD4; CD80 and CD52; CD80 and VEGF; CD80 and DR5; CD80 and VEGFR2; CD22 and CD20; CD22 and CD80; CD22 and CD40; CD22 and CD23; CD22 and CD33; CD22 and CD74; CD22 and CD19; CD22 and DR5; CD22 and DR4; CD22 and VEGF; CD22 and CD52; CD30 and CD20; CD30 and CD22; CD30 and CD23; CD30 and CD40; CD30 and VEGF; CD30 and CD74; CD30 and CD19; CD30 and DR5; CD30 and DR4; CD30 and VEGFR2; CD30 and CD52; CD30 and CD4; CD138 and RANKL; CD33 and FTL3; CD33 and VEGF; CD33 and VEGFR2; CD33 and CD44; CD33 and DR4; CD33 and DR5; DR4 and CD137; DR4 and IGF1,2; DR4 and IGF1R; DR4 and DR5; DR5 and CD40; DR5 and CD137; DR5 and CD20; DR5 and EGFR; DR5 and IGF1,2; DR5 and IGFR, DR5 and HER-2, and EGFR and DLL4. Other target combinations include one or more members of the EGF/erb-2/erb-3 family.

Other targets (one or more) involved in oncological diseases that the multivalent antibody analogs disclosed herein may bind include, but are not limited to those selected from the group consisting of: CD52, CD20, CD19, CD3, CD4, CD8, BMP6, IL12A, IL1A, IL1B, 1L2, IL24, INHA, TNF, TNFSF10, BMP6, EGF, FGF1, FGF10, FGF11, FGF12, FGF13, FGF14, FGF16, FGF17, FGF18, FGF19, FGF2, FGF20, FGF21, FGF22, FGF23, FGF3, FGF4, FGF5, FGF6, FGF7, FGF8, FGF9, GRP, IGF1, IGF2, IL12A, IL1A, IL1B, IL2, INHA, TGFA, TGFB1, TGFB2, TGFB3, VEGF, CDK2, FGF10, FGF18, FGF2, FGF4, FGF7, IGF1R, IL2, BCL2, CD164, CDKN1A, CDKN1B, CDKN1C, CDKN2A, CDKN2B, CDKN2C, CDKN3, GNRH1, IGFBP6, IL1A, IL1B, ODZ1, PAWR, PLG, TGFBIII, AR, BRCA1, CDK3, CDK4, CDK5, CDK6, CDK7, CDK9, E2F1, EGFR, ENO1, ERBB2, ESR1, ESR2, IGFBP3, IGFBP6, IL2, INSL4, MYC, NOX5, NR6A1, PAP, PCNA, PRKCQ, PRKD1, PRL, TP53, FGF22, FGF23, FGF9, IGFBP3, IL2, INHA, KLK6, TP53, CHGB, GNRH1, IGF1, IGF2, INHA, INSL3, INSL4, PRL, KLK6, SHBG, NR1D1, NR1H3, NR113, NR2F6, NR4A3, ESR1, ESR2, NR0B1, NR0B2, NR1D2, NR1H2, NR1H4, NR112, NR2C1, NR2C2, NR2E1, NR2E3, NR2F1, NR2F2, NR3C1, NR3C2, NR4A1, NR4A2, NR5A1, NR5A2, NR6 μl, PGR, RARB, FGF1, FGF2, FGF6, KLK3, KRT1, APOC1, BRCA1, CHGA, CHGB, CLU, COL1A1, COL6A1, EGF, ERBB2, ERK8, FGF1, FGF10, FGF11, FGF13, FGF14, FGF16, FGF17, FGF18, FGF2, FGF20, FGF21, FGF22, FGF23, FGF3, FGF4, FGF5, FGF6, FGF7, FGF8, FGF9, GNRH1, IGF1, IGF2, IGFBP3, IGFBP6, IL12A, IL1A, IL1B, 1L2, IL24, INHA, INSL3, INSL4, KLK10, KLK12, KLK13, KLK14, KLK15, KLK3, KLK4, KLK5, KLK6, KLK9, MMP2, MMP9, MSMB, NTN4, ODZ1, PAP, PLAU, PRL, PSAP, SERPINA3, SHBG, TGFA, TIMP3, CD44, CDH1, CDH10, CDH19, CDH20, CDH7, CDH9, CDH1, CDH10, CDH13, CDH18, CDH19, CDH20, CDH7, CDH8, CDH9, ROBO2, CD44, ILK, ITGA1, APC, CD164, COL6A1, MTSS1, PAP, TGFBIII, AGR2, AIG1, AKAP1, AKAP2, CANT1, CAVI, CDH12, CLDN3, CLN3, CYB5, CYC1, DAB21P, DES, DNCL1, ELAC2, ENO2, ENO3, FASN, FLJ12584, FLJ25530, GAGEB1, GAGEC1, GGT1, GSTP1, HIP1, HUMCYT2A, IL29, K6HF, KAI1, KRT2A, MIB1, PART1, PATE, PCA3, PIAS2, PIK3CG, PPID, PR1, PSCA, SLC2A2, SLC33 μl, SLC43 μl, STEAP, STEAP2, TPM1, TPM2, TRPC6, ANGPT1, ANGPT2, ANPEP, ECGF1, EREG, FGF1, FGF2, FIGF, FLT1, JAG1, KDR, LAMA5, NRP1, NRP2, PGF, PLXDCI, STAB 1, VEGF, VEGFC, ANGPTL3, BA11, COL4A3, IL8, LAMA5, NRP1, NRP2, STAB 1, ANGPTL4, PECAM1, PF4, PROK2, SERPINF1, TNFAIP2, CCL11, CCL2, CXCL1, CXCL10, CXCL3, CXCL5, CXCL6, CXCL9, IFNA1, IFNB1, IFNG, IL1B, 1L6, MDK, EDG1, EFNA1, EFNA3, EFNB2, EGF, EPHB4, FGFR3, HGF, IGF1, ITGB3, PDGFA, TEK, TGFA, TGFB1, TGFB2, TGFBR1, CCL2, CDH5, COL1A1, EDG1, ENG, ITGAV, ITGB3, THBS1, THBS2, BAD, BAG1, BCL2, CCNA1, CCNA2, CCND1, CCNE1, CCNE2, CDH1 (E-cadherin), CDKN1B (p27Kip1), CDKN2A (p161NK4a), COL6A1, CTNNB1 (b-catenin), CTSB (cathepsin B), ERBB2 (Her-2), ESR1, ESR2, F3 (TF), FOSL1 (FRA-1), GATA3, GSN (Gelsolin), IGFBP2, IL2RA, IL6, IL6R, IL6ST (glycoprotein 130), ITGA6 (a6 integrin), JUN, KLK5, KRT19, MAP2K7 (c-Jun), MKI67 (Ki-67), NGFB (GF), NGFR, NME1 (M23A), PGR, PLAU (uPA), PTEN, SERPINB5 (maspin), SERPINE1 (PAI-1), TGFA, THBS1 (thrombospondin-1), TIE (Tie-1), TNFRSF6 (Fas), TNFSF6 (FasL), TOP2A (topoisomerase Iia), TP53, AZGP1 (zinc-a-glycoprotein), BPAG1 (plectin), CDKN1A (p21Wap1/Cip1), CLDN7 (claudin-7), CLU (clusterin), ERBB2 (Her-2), FGF1, FLRT1 (fibronectin), GABRP (GABAa), GNAS1, 1D2, ITGA6 (a6 integrin), ITGB4 (b 4 integrin), KLF5 (GC Box BP), KRT19 (Keratin 19), KRTHB6 (hair-specific type II keratin), MACMARCKS, MT3 (metallothionectin-111), MUC1 (mucin), PTGS2 (COX-2), RAC2 (p21Rac2), S100A2, SCGB1D2 (lipophilin B), SCGB2A1 (mammaglobin 2), SCGB2A2 (mammaglobin 1), SPRR1B (Spr1), THBS1, THBS2, THBS4, and TNFAIP2 (B94), RON, c-Met, CD64, DLL4, PLGF, CTLA4, phophatidylserine, ROBO4, CD80, CD22, CD40, CD23, CD28, CD80, CD55, CD38, CD70, CD74, CD30, CD138, CD56, CD33, CD2, CD137, DR4, DRS, RANKL, VEGFR2, PDGFR, VEGFR1, MTSP1, MSP, EPHB2, EPHA1, EPHA2, EpCAM, PGE2, NKG2D, LPA, SIP, APRIL, BCMA, MAPG, FLT3, PDGFR alpha, PDGFR beta, ROR1, PSMA, PSCA, SCD1, and CD59.

Monoclonal antibody therapy has become an important therapeutic modality for treating autoimmune and inflammatory disorders (Chan & Carter, 2010, Nature Reviews Immunology 10:301-316; Reichert et al., 2005, Nature Biotechnology 23[9]:1073-1078). Many proteins have been implicated in general autoimmune and inflammatory responses, and thus may be targeted by the inventive multivalent antibody analogs of the invention. Autoimmune and inflammatory targets include but are not limited to C5, CCL1 (1-309), CCL11 (eotaxin), CCL13 (mcp-4), CCL15 (MIP-1d), CCL16 (HCC-4), CCL17 (TARC), CCL18 (PARC), CCL19, CCL2 (mcp-1), CCL20 (MIP-3a), CCL21 (MIP-2), CCL23 (MPIF-1), CCL24 (MPIF-2/eotaxin-2), CCL25 (TECK), CCL26, CCL3 (MIP-1a), CCL4 (MIP-1b), CCL5 (RANTES), CCL7 (mcp-3), CCL8 (mcp-2), CXCL1, CXCL10 (1P-10), CXCL11 (1-TAC/IP-9), CXCL12 (SDF1), CXCL13, CXCL14, CXCL2, CXCL3, CXCL5 (ENA-78/LIX), CXCL6 (GCP-2), CXCL9, IL13, IL8, CCL13 (mcp-4), CCR1, CCR2, CCR3, CCR4, CCR5, CCR6, CCR7, CCR8, CCR9, CX3CR1, IL8RA, XCR1 (CCXCR1), IFNA2, IL10, IL13, IL17C, IL1A, IL1B, 1L1F10, IL1F5, IL1F6, IL1F7, IL1F8, IL1F9, IL22, IL5, IL8, IL9, LTA, LTB, MIF, SCYE1 (endothelial Monocyte-activating cytokine), SPP1, TNF, TNFSF5, IFNA2, IL10RA, IL10RB, IL13, IL13RA1, IL5RA, IL9, IL9R, ABCF₁, BCL6, C3, C4A, CEBPB, CRP, ICEBERG, IL1R1, IL1RN, IL8RB, LTB4R, TOLLIP, FADD, IRAK1, IRAK2, MYD88, NCK2, TNFAIP3, TRADD, TRAF1, TRAF2, TRAF3, TRAF4, TRAF5, TRAF6, ACVR1, ACVR1B, ACVR2, ACVR2B, ACVRL1, CD28, CD3E, CD3G, CD3Z, CD69, CD80, CD86, CNR1, CTLA4, CYSLTR1, FCER1A, FCER2, FCGR3A, GPR44, HAVCR2, OPRD1, P2RX7, TLR2, TLR3, TLR4, TLR5, TLR6, TLR7, TLR8, TLR9, TLR10, BLR1, CCL1, CCL2, CCL3, CCL4, CCL5, CCL7, CCL8, CCL11, CCL13, CCL15, CCL16, CCL17, CCL18, CCL19, CCL20, CCL21, CCL22, CCL23, CCL24, CCL25, CCR1, CCR2, CCR3, CCR4, CCR5, CCR6, CCR7, CCR8, CCR9, CX3CL1, CX3CR1, CXCL1, CXCL2, CXCL3, CXCL5, CXCL6, CXCL10, CXCL11, CXCL12, CXCL13, CXCR4, GPR2, SCYE1, SDF2, XCL1, XCL2, XCR1, AMH, AMHR2, BMPR1A, BMPR1B, BMPR2, C19orf10 (IL27w), CER1, CSF1, CSF2, CSF3, DKFZp451J0118, FGF2, GFI1, IFNA1, IFNB1, IFNG, IGF1, IL1A, IL1B, IL1R1, IL1R2, IL2, IL2RA, IL2RB, IL2RG, IL3, IL4, IL4R, IL5, IL5RA, IL6, IL6R, IL6ST, IL7, IL8, IL8RA, IL8RB, IL9, IL9R, IL10, IL10RA, IL10RB, IL11, IL12RA, IL12A, IL12B, IL12RB1, IL12RB2, IL13, IL13RA1, IL13RA2, IL15, IL15RA, IL16, IL17, IL17R, IL18, IL18R1, IL19, IL20, KITLG, LEP, LTA, LTB, LTB4R, LTB4R2, LTBR, MIF, NPPB, PDGFB, TBX21, TDGF1, TGFA, TGFB1, TGFB111, TGFB2, TGFB3, TGFB1, TGFBR1, TGFBR2, TGFBR3, TH1L, TNF, TNFRSF1A, TNFRSF1B, TNFRSF7, TNFRSF8, TNFRSF9, TNFRSF11A, TNFRSF21, TNFSF4, TNFSF5, TNFSF6, TNFSF11, VEGF, ZFPM2, and RNF110 (ZNF144).

Exemplary co-targets for autoimmune and inflammatory disorders include but are not limited to IL-1 and TNFalpha, IL-6 and TNFalpha, IL-6 and IL-1, IgE and IL-13, IL-1 and IL-13, IL-4 and IL-13, IL-5 and IL-13, IL-9 and IL-13, CD19 and FcγRIIb, and CD79 and FcγRIIb.

Multivalent antibody analogs of the invention with specificity for the following pairs of targets to treat inflammatory disease are contemplated: TNF and IL-17A; TNF and RANKL; TNF and VEGF; TNF and SOST; TNF and DKK; TNF and alphaVbeta3; TNF and NGF; TNF and IL-23p19; TNF and IL-6; TNF and SOST; TNF and IL-6R; TNF and CD-20; IgE and IL-13; IL-13 and IL23p19; IgE and IL-4; IgE and IL-9; IgE and IL-9; IgE and IL-13; IL-13 and IL-9; IL-13 and IL-4; IL-13 and IL-9; IL-13 and IL-9; IL-13 and IL-4; IL-13 and IL-23p19; IL-13 and IL-9; IL-6R and VEGF; IL-6R and IL-17A; IL-6R and RANKL; IL-17A and IL-1 beta; IL-1 beta and RANKL; IL-1beta and VEGF; RANKL and CD-20; IL-1alpha and IL-1 beta; IL-1 alpha and IL-1beta.

Pairs of targets that the multivalent antibody analogs described herein can bind and be useful to treat asthma may be determined. In an embodiment, such targets include, but are not limited to, IL-13 and IL-1 beta, since IL-1 beta is also implicated in inflammatory response in asthma; IL-13 and cytokines and chemokines that are involved in inflammation, such as IL-13 and IL-9; IL-13 and IL-4; IL-13 and IL-5; IL-13 and IL-25; IL-13 and TARC; IL-13 and MDC; IL-13 and MIF; IL-13 and TGF-13; IL-13 and LHR agonist; IL-13 and CL25; IL-13 and SPRR2a; IL-13 and SPRR2b; and IL-13 and ADAMS. The inventive multivalent antibody analogs herein may have specificity for one or more targets involved in asthma selected from the group consisting of CSF1 (MCSF), CSF2 (GM-CSF), CSF3 (GCSF), FGF2, IFNA1, IFNB1, IFNG, histamine and histamine receptors, IL1A, IL1 B, IL2, IL3, IL4, IL5, IL6, IL7, IL8, IL9, IL10, IL11, IL12A, IL12B, IL13, IL14, IL15, IL16, IL17, IL18, IL19, KITLG, PDGFB, IL2RA, IL4R, IL5RA, IL8RA, IL8RB, IL12RB1, IL12RB2, IL13RA1, IL13RA2, IL18R1, TSLP, CCLi, CCL2, CCL3, CCL4, CCL5, CCL7, CCL8, CCL13, CCL17, CCL18, CCL19, CCL20, CCL22, CCL24, CX3CL1, CXCL1, CXCL2, CXCL3, XCLi, CCR2, CCR3, CCR4, CCR5, CCR6, CCR7, CCR8, CX3CR1, GPR2, XCR1, FOS, GATA3, JAK1, JAK3, STATE, TBX21, TGFB1, TNF, TNFSF6, YY1, CYSLTR1, FCER1A, FCER2, LTB4R, TB4R2, LTBR, and Chitinase.

Pairs of targets involved in rheumatoid arthritis (RA) may be co-targeted by the invention, including but not limited to TNF and IL-18; TNF and IL-12; TNF and IL-23; TNF and 1L-1beta; TNF and MIF; TNF and IL-17; and TNF and IL-15.

Antigens that may be targeted in order to treat systemic lupus erythematosus (SLE) by the inventive multivalent antibody analogs herein include but are not limited to CD-20, CD-22, CD-19, CD28, CD4, CD80, HLA-DRA, IL10, IL2, IL4, TNFRSF5, TNFRSF6, TNFSF5, TNFSF6, BLR1, HDAC4, HDAC5, HDAC7A, HDAC9, ICOSL, IGBP1, MS4A1, RGSI, SLA2, CD81, IFNB1, IL10, TNFRSF5, TNFRSF7, TNFSF5, AICDA, BLNK, GALNAC4S-6ST, HDAC4, HDAC5, HDAC7A, HDAC9, IL10, IL11, IL4, INHA, INHBA, KLF6, TNFRSF7, CD28, CD38, CD69, CD80, CD83, CD86, DPP4, FCER2, IL2RA, TNFRSF8, TNFSF7, CD24, CD37, CD40, CD72, CD74, CD79A, CD79B, CR2, ILIR2, ITGA2, ITGA3, MS4A1, ST6GALI, CDIC, CHSTIO, HLA-A, HLA-DRA, and NT5E.; CTLA4, B7.1, B7.2, BIyS, BAFF, C5, IL-4, IL-6, IL-10, IFN-α, and TNF-α.

The inventive multivalent antibody analogs herein may target antigens for the treatment of multiple sclerosis (MS), including but not limited to IL-12, TWEAK, IL-23, CXCL13, CD40, CD40L, IL-18, VEGF, VLA-4, TNF, CD45RB, CD200, IFNgamma, GM-CSF, FGF, C5, CD52, and CCR2. An embodiment includes co-engagement of anti-IL-12 and TWEAK for the treatment of MS.

One aspect of the invention pertains to inventive multivalent antibody analogs capable of binding one or more targets involved in sepsis, in an embodiment two targets, selected from the group consisting TNF, IL-1, MIF, IL-6, IL-8, IL-18, IL-12, IL-23, FasL, LPS, Toll-like receptors, TLR-4, tissue factor, MIP-2, ADORA2A, CASP1, CASP4, IL-10, IL-1B, NFκB1, PROC, TNFRSFIA, CSF3, CCR3, ILIRN, MIF, NFκB1, PTAFR, TLR2, TLR4, GPR44, HMOX1, midkine, IRAK1, NFκB2, SERPINA1, SERPINE1, and TREM1.

In some cases, inventive multivalent antibody analogs herein may be directed against antigens for the treatment of infectious diseases.

The inventive multivalent antibody analogs may be screened using a variety of in vitro methods, including but not limited to those that use binding assays, cell-based assays, and selection technologies. Automation and high-throughput screening technologies may be utilized in the screening procedures. Screening may employ the use of a fusion partner or label. The use of fusion partners has been discussed above. By “labeled” herein is meant that the inventive multivalent antibody analogs disclosed herein have one or more elements, isotopes, or chemical compounds attached to enable the detection in a screen. In general, labels fall into three classes: a) immune labels, which may be an epitope incorporated as a fusion partner that is recognized by an antibody, b) isotopic labels, which may be radioactive or heavy isotopes, and c) small molecule labels, which may include fluorescent and colorimetric dyes, or molecules such as biotin that enable other labeling methods. Labels may be incorporated into the compound at any position and may be incorporated in vitro or in vivo during protein expression.

In certain embodiments, the functional and/or biophysical properties of the inventive multivalent antibody analogs are screened in an in vitro assay. In vitro assays may allow a broad dynamic range for screening properties of interest. Particularly relevant for the present invention, the inventive multivalent antibody analogs may be tested for their affinity for one or more antigens. Properties that may be screened include but are not limited to stability, solubility, and affinity for Fc ligands, for example FcγRs. Multiple properties may be screened simultaneously or individually. Proteins may be purified or unpurified, depending on the requirements of the assay. In one embodiment, the screen is a qualitative or quantitative binding assay for binding of inventive multivalent antibody analogs to a protein or nonprotein molecule that is known or thought to bind the inventive multivalent antibody analog. In one embodiment, the screen is a binding assay for measuring binding to the target antigen. In an alternate embodiment, the screen is an assay for binding of inventive multivalent antibody analogs to an Fc ligand, including but are not limited to the family of FcγRs, the neonatal receptor FcRn, the complement protein C1q, and the bacterial proteins A and G. Said Fc ligands may be from any organism. In one embodiment, Fc ligands are from humans, mice, rats, rabbits, and/or monkeys. Binding assays can be carried out using a variety of methods known in the art, including but not limited to FRET (Fluorescence Resonance Energy Transfer) and BRET (Bioluminescence Resonance Energy Transfer)-based assays, AlphaScreen™ (Amplified Luminescent Proximity Homogeneous Assay), Scintillation Proximity Assay, ELISA (Enzyme-Linked Immunosorbent Assay), SPR (Surface Plasmon Resonance, also known as BIACORE®), isothermal titration calorimetry, differential scanning calorimetry, gel electrophoresis, and chromatography including gel filtration. These and other methods may take advantage of some fusion partner or label of the inventive multivalent antibody analog. Assays may employ a variety of detection methods including but not limited to chromogenic, fluorescent, luminescent, or isotopic labels.

The biophysical properties of the inventive multivalent antibody analogs, for example stability and solubility, may be tested using a variety of methods known in the art. Protein stability may be determined by measuring the thermodynamic equilibrium between folded and unfolded states. For example, inventive multivalent antibody analogs disclosed herein may be unfolded using chemical denaturant, heat, or pH, and this transition may be monitored using methods including but not limited to circular dichroism spectroscopy, fluorescence spectroscopy, absorbance spectroscopy, NMR spectroscopy, calorimetry, and proteolysis. As will be appreciated by those skilled in the art, the kinetic parameters of the folding and unfolding transitions may also be monitored using these and other techniques. The solubility and overall structural integrity of an inventive multivalent antibody analog may be quantitatively or qualitatively determined using a wide range of methods that are known in the art. Methods which may find use for characterizing the biophysical properties of inventive multivalent antibody analogs disclosed herein include gel electrophoresis, isoelectric focusing, capillary electrophoresis, chromatography such as size exclusion chromatography, ion-exchange chromatography, and reversed-phase high performance liquid chromatography, peptide mapping, oligosaccharide mapping, mass spectrometry, ultraviolet absorbance spectroscopy, fluorescence spectroscopy, circular dichroism spectroscopy, isothermal titration calorimetry, differential scanning calorimetry, analytical ultra-centrifugation, dynamic light scattering, proteolysis, and cross-linking, turbidity measurement, filter retardation assays, immunological assays, fluorescent dye binding assays, protein-staining assays, microscopy, and detection of aggregates via ELISA or other binding assay. Structural analysis employing X-ray crystallographic techniques and NMR spectroscopy may also find use. In one embodiment, stability and/or solubility may be measured by determining the amount of protein solution after some defined period of time. In this assay, the protein may or may not be exposed to some extreme condition, for example elevated temperature, low pH, or the presence of denaturant. Because function typically requires a stable, soluble, and/or well-folded/structured protein, the aforementioned functional and binding assays also provide ways to perform such a measurement. For example, a solution comprising an inventive multivalent antibody analog could be assayed for its ability to bind target antigen, then exposed to elevated temperature for one or more defined periods of time, then assayed for antigen binding again. Because unfolded and aggregated protein is not expected to be capable of binding antigen, the amount of activity remaining provides a measure of the inventive multivalent antibody analog's stability and solubility.

In certain embodiments, the inventive multivalent antibody analogs may be tested using one or more cell-based or in vitro assays. For such assays, inventive multivalent antibody analogs, purified or unpurified, are typically added exogenously such that cells are exposed to inventive multivalent antibody analogs described herein. These assays are typically, but not always, based on the biology of the ability of the inventive multivalent antibody analog to bind to the target antigen and mediate some biochemical event, for example effector functions like cellular lysis, phagocytosis, ligand/receptor binding inhibition, inhibition of growth and/or proliferation, inhibition of calcium release and/or signaling, apoptosis and the like. Such assays often involve monitoring the response of cells to inventive multivalent antibody analog, for example cell survival, cell death, cellular phagocytosis, cell lysis, change in cellular morphology, or transcriptional activation such as cellular expression of a natural gene or reporter gene. For example, such assays may measure the ability of inventive multivalent antibody analogs to elicit cell killing, for example ADCC, ADCP, and CDC. Assays that measure cellular killing that is mediated by co-engagement of antigens are particularly relevant for the invention. For some assays additional cells or components, that is in addition to the target cells, may need to be added, for example serum complement, or effector cells such as peripheral blood monocytes (PBMCs), NK cells, macrophages, T cells, and the like. Such additional cells may be from any organism, e.g., humans, mice, rat, rabbit, and monkey. Crosslinked or monomeric antibodies may cause apoptosis of certain cell lines expressing the antibody's target antigen, or they may mediate attack on target cells by immune cells which have been added to the assay. Methods for monitoring cell death or viability are known in the art, and include the use of dyes, fluorophores, immunochemical, cytochemical, and radioactive reagents. For example, caspase assays or annexin-flourconjugates may enable apoptosis to be measured, and uptake or release of radioactive substrates (e.g. Chromium-51 release assays) or the metabolic reduction of fluorescent dyes such as alamar blue may enable cell growth, proliferation or activation to be monitored. In one embodiment, the DELFIA EuTDA-based cytotoxicity assay (Perkin Elmer, Mass.) is used. Alternatively, dead or damaged target cells may be monitored by measuring the release of one or more natural intracellular proteins, for example lactate dehydrogenase. Transcriptional activation may also serve as a method for assaying function in cell-based assays. In this case, response may be monitored by assaying for natural genes or proteins which may be upregulated or down-regulated, for example the release of certain interleukins may be measured, or alternatively readout may be via a luciferase or GFP-reporter construct. Cell-based assays may also involve the measure of morphological changes of cells as a response to the presence of an inventive multivalent antibody analog. Cell types for such assays may be prokaryotic or eukaryotic, and a variety of cell lines that are known in the art may be employed. Alternatively, cell-based screens are performed using cells that have been transformed or transfected with nucleic acids encoding the inventive multivalent antibody analogs.

The biological properties of the inventive multivalent antibody analogs disclosed herein may be characterized in cell, tissue, and whole organism experiments. As is known in the art, drugs are often tested in animals, including but not limited to mice, rats, rabbits, dogs, cats, pigs, and monkeys, in order to measure a drug's efficacy for treatment against a disease or disease model, or to measure a drug's pharmacokinetics, toxicity, and other properties. Said animals may be referred to as disease models. With respect to the inventive multivalent antibody analogs disclosed herein, a particular challenge arises when using animal models to evaluate the potential for in-human efficacy of candidate polypeptides—this is due, at least in part, to the fact that inventive multivalent antibody analogs that have a specific effect on the affinity for a human Fc receptor may not have a similar affinity effect with the orthologous animal receptor. These problems can be further exacerbated by the inevitable ambiguities associated with correct assignment of true orthologues (Mechetina et al., 2002, Immunogenetics 54:463-468), and the fact that some orthologues simply do not exist in the animal. Therapeutics are often tested in mice, including but not limited to nude mice, Rag-deficient mice, SCID mice, xenograft mice, and transgenic mice (including knockins and knockouts). For example, an inventive multivalent antibody analog of the present invention that is intended as an anti-cancer therapeutic may be tested in a mouse cancer model, for example a xenograft mouse. In this method, a tumor or tumor cell line is grafted onto or injected into a mouse, and subsequently the mouse is treated with the therapeutic to determine the ability of the drug to reduce or inhibit cancer growth and metastasis. Therapeutic inventive multivalent antibody analogs herein can be tested in mouse strains NZB, NOD, BXSB, MRL/Ipr, K/BxN and transgenics (including knockins and knockouts). Such mice can develop various autoimmune conditions that resemble human organ specific, systemic autoimmune or inflammatory disease pathologies such as systemic lupus erythematosus (SLE) and rheumatoid arthritis (RA). For example, an inventive multivalent antibody analog disclosed herein intended for autoimmune diseases may be tested in such mouse models by treating the mice to determine the ability of the inventive multivalent antibody analog to reduce or inhibit the development of the disease pathology. Because of the incompatibility between the mouse and human Fey receptor system, an alternative approach is to use a murine SCID model in which immune deficient mice are engrafted with human PBLs or PBMCs (huPBL-SCID, huPBMC-SCID) providing a semi-functional human immune system with human effector cells and Fc receptors. Other organisms, e.g., mammals, may also be used for testing. For example, because of their genetic similarity to humans, monkeys can be suitable therapeutic models, and thus may be used to test the efficacy, toxicity, pharmacokinetics, or other property of the inventive multivalent antibody analogs disclosed herein. Tests of the inventive multivalent antibody analogs disclosed herein in humans are ultimately required for approval as drugs, and thus of course these experiments are contemplated. Thus the inventive multivalent antibody analogs disclosed herein may be tested in humans to determine their therapeutic efficacy, toxicity, pharmacokinetics, and/or other clinical properties.

In some embodiments, inventive multivalent antibody analogs disclosed herein may be assessed for efficacy in clinically relevant animal models of various human diseases. In many cases, relevant models include various transgenic animals for specific antigens and receptors.

In certain embodiments, the testing of inventive multivalent antibody analogs may include study of efficacy in primates (e.g. cynomolgus monkey model) to facilitate the evaluation of depletion of specific target cells harboring the target antigen. Additional primate models include but are not limited to use of the rhesus monkey to assess inventive multivalent antibody analogs in therapeutic studies of autoimmune, transplantation and cancer.

Toxicity studies are performed to determine drug related-effects that cannot be evaluated in standard pharmacology profiles, or occur only after repeated administration of the agent. Most toxicity tests are performed in two species—a rodent and a non-rodent—to ensure that any unexpected adverse effects are not overlooked before new therapeutic entities are introduced into man. In general, these models may measure a variety of toxicities including genotoxicity, chronic toxicity, immunogenicity, reproductive/developmental toxicity and carcinogenicity. Included within the aforementioned parameters are standard measurement of food consumption, bodyweight, antibody formation, clinical chemistry, and macro- and microscopic examination of standard organs/tissues (e.g. cardiotoxicity). Additional parameters of measurement are injection site trauma and the measurement of neutralizing antibodies, if any. Traditionally, monoclonal antibody therapeutics, naked or conjugated, is evaluated for cross-reactivity with normal tissues, immunogenicity/antibody production, conjugate or linker toxicity and “bystander” toxicity of radiolabelled species. Nonetheless, such studies may have to be individualized to address specific concerns and following the guidance set by ICH S6 (Safety studies for biotechnological products, also noted above). As such, the general principles are that the products are sufficiently well characterized, impurities/contaminants have been removed, that the test material is comparable throughout development, and that GLP compliance is maintained.

The pharmacokinetics (PK) of the inventive multivalent antibody analogs disclosed herein may be studied in a variety of animal systems, with the most relevant being non-human primates such as the cynomolgus and rhesus monkeys. Single or repeated i.v./s.c. administrations over a dose range of 6000-fold (0.05-300 mg/kg) can be evaluated for half-life (days to weeks) using plasma concentration and clearance. Volume of distribution at a steady state and level of systemic absorbance can also be measured. Examples of such parameters of measurement generally include maximum observed plasma concentration (Cmax), the time to reach Cmax(Tmax), the area under the plasma concentration-time curve from time 0 to infinity [AUC(0-inf] and apparent elimination half-life (T½). Additional measured parameters could include compartmental analysis of concentration-time data obtained following i.v. administration and bioavailability.

Pharmacodynamic studies may include, but are not limited to, targeting specific cells or blocking signaling mechanisms, measuring inhibition of antigen-specific antibodies etc. The inventive multivalent antibody analogs disclosed herein may target particular effector cell populations and thereby be direct drugs to induce certain activities to improve potency or to increase penetration into a particularly favorable physiological compartment. Such pharmacodynamic effects may be demonstrated in animal models or in humans

The inventive multivalent antibody analogs disclosed herein may find use in a wide range of products. In one embodiment an inventive multivalent antibody analog disclosed herein comprise a therapeutic, a diagnostic, or a research reagent. The inventive multivalent antibody analogs may find use in a composition that is monoclonal or polyclonal. The inventive multivalent antibody analogs disclosed herein may be used for therapeutic purposes. As will be appreciated by those in the art, the inventive multivalent antibody analogs disclosed herein may be used for any therapeutic purpose that antibodies, Fc fusions, and the like may be used for. The inventive multivalent antibody analogs may be administered to a patient to treat disorders including but not limited to cancer, infectious diseases, autoimmune and inflammatory diseases. A “patient” for the purposes disclosed herein includes both humans and other animals, e.g., other mammals. Thus the inventive multivalent antibody analogs disclosed herein have both human therapy and veterinary applications. The term “treatment” or “treating” as disclosed herein is meant to include therapeutic treatment, as well as prophylactic, or suppressive measures for a disease or disorder. Thus, for example, successful administration of an inventive multivalent antibody analog prior to onset of the disease results in treatment of the disease. As another example, successful administration of an optimized inventive multivalent antibody analog after clinical manifestation of the disease to combat the symptoms of the disease comprises treatment of the disease. “Treatment” and “treating” also encompasses administration of an optimized inventive multivalent antibody analog after the appearance of the disease in order to eradicate the disease. Successful administration of an agent after onset and after clinical symptoms have developed, with possible abatement of clinical symptoms and perhaps amelioration of the disease, comprises treatment of the disease. Those “in need of treatment” include mammals already having the disease or disorder, as well as those prone to having the disease or disorder, including those in which the disease or disorder is to be prevented.

In one embodiment, the inventive multivalent antibody analogs disclosed herein are administered to a patient having a disease involving inappropriate expression of a protein or other molecule. Within the scope disclosed herein this is meant to include diseases and disorders characterized by aberrant proteins, due for example to alterations in the amount of a protein present, protein localization, posttranslational modification, conformational state, the presence of a mutant or pathogen protein, etc. Similarly, the disease or disorder may be characterized by alterations molecules including but not limited to polysaccharides and gangliosides. An overabundance may be due to any cause, including but not limited to overexpression at the molecular level, prolonged or accumulated appearance at the site of action, or increased activity of a protein relative to normal. Included within this definition are diseases and disorders characterized by a reduction of a protein. This reduction may be due to any cause, including but not limited to reduced expression at the molecular level, shortened or reduced appearance at the site of action, mutant forms of a protein, or decreased activity of a protein relative to normal. Such an overabundance or reduction of a protein can be measured relative to normal expression, appearance, or activity of a protein, and said measurement may play an important role in the development and/or clinical testing of the inventive multivalent antibody analogs disclosed herein.

The inventive multivalent antibody analogs herein may be used to treat cancer. By “cancer” and “cancerous” herein refer to or describe the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia or lymphoid malignancies.

More particular examples of such cancers include hematologic malignancies, such as Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia; tumors of the central nervous system such as glioma, glioblastoma, neuroblastoma, astrocytoma, medulloblastoma, ependymoma, and retinoblastoma; solid tumors of the head and neck (e.g. nasopharyngeal cancer, salivary gland carcinoma, and esophagael cancer), lung (e.g. small-cell lung cancer, non-small cell lung cancer, adenocarcinoma of the lung and squamous carcinoma of the lung), digestive system (e.g. gastric or stomach cancer including gastrointestinal cancer, cancer of the bile duct or biliary tract, colon cancer, rectal cancer, colorectal cancer, and anal carcinoma), reproductive system (e.g. testicular, penile, or prostate cancer, uterine, vaginal, vulval, cervical, ovarian, and endometrial cancer), skin (e.g. melanoma, basal cell carcinoma, squamous cell cancer, actinic keratosis), liver (e.g. liver cancer, hepatic carcinoma, hepatocellular cancer, and hepatoma), bone (e.g. osteoclastoma, and osteolytic bone cancers) additional tissues and organs (e.g. pancreatic cancer, bladder cancer, kidney or renal cancer, thyroid cancer, breast cancer, cancer of the peritoneum, and Kaposi's sarcoma), and tumors of the vascular system (e.g. angiosarcoma and hemagiopericytoma).

The inventive multivalent antibody analogs disclosed herein may be used to treat autoimmune diseases. By “autoimmune diseases” herein include allogenic islet graft rejection, alopecia greata, ankylosing spondylitis, antiphospholipid syndrome, autoimmune Addison's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune disfunction syndrome, chronic inflammatory demyelinating polyneuropathy, Churg-Strauss syndrome, cicatrical pemphigoid, CREST syndrome, cold agglutinin disease, Crohn's disease, dermatomyositis, discoid lupus, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen plantus, lupus erthematosis, Meniere's disease, mixed connective tissue disease, multiple sclerosis, type 1 diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobinulinemia, primary biliary cirrhosis, psoriasis, psoriatic arthritis, Reynauld's phenomenon, Reiter's syndrome, rheumatoid arthritis, sarcoidosis, scleroderma, Sjorgen's syndrome, solid organ transplant rejection, stiff-man syndrome, systemic lupus erythematosus, takayasu arteritis, temporal arteristis/giant cell arteritis, thrombotic thrombocytopenia purpura, ulcerative colitis, uveitis, vasculitides such as dermatitis herpetiformis vasculitis, vitiligo, and Wegner's granulomatosis.

The inventive multivalent antibody analogsdisclosed herein may be used to treat inflammatory disorders. By “inflammatory disorders” herein include acute respiratory distress syndrome (ARDS), acute septic arthritis, adjuvant arthritis, juvenile idiopathic arthritis, allergic encephalomyelitis, allergic rhinitis, allergic vasculitis, allergy, asthma, atherosclerosis, chronic inflammation due to chronic bacterial or viral infections, chronic obstructive pulmonary disease (COPD), coronary artery disease, encephalitis, inflammatory bowel disease, inflammatory osteolysis, inflammation associated with acute and delayed hypersensitivity reactions, inflammation associated with tumors, peripheral nerve injury or demyelinating diseases, inflammation associated with tissue trauma such as burns and ischemia, inflammation due to meningitis, multiple organ injury syndrome, pulmonary fibrosis, sepsis and septic shock, Stevens-Johnson syndrome, undifferentiated arthropy, and undifferentiated spondyloarthropathy.

Some autoimmune and inflammatory diseases that may be targeted by the inventive multivalent antibody analogs disclosed herein include Systemic Lupus Erythematosus, Rheumatoid arthritis, Sjogren's syndrome, Multiple sclerosis, Idiopathic thrombocytopenic purpura (ITP), Graves disease, Inflammatory bowel disease, Psoriasis, Type I diabetes, and Asthma.

The inventive multivalent antibody analogs herein may be used to treat infectious diseases. By “infectious diseases” herein include diseases caused by pathogens such as viruses, bacteria, fungi, protozoa, and parasites. Infectious diseases may be caused by viruses including adenovirus, cytomegalovirus, dengue, Epstein-Barr, hanta, hepatitis A, hepatitis B, hepatitis C, herpes simplex type I, herpes simplex type II, human immunodeficiency virus, (HIV), human papilloma virus (HPV), influenza, measles, mumps, papova virus, polio, respiratory syncytial virus, rinderpest, rhinovirus, rotavirus, rubella, SARS virus, smallpox, viral meningitis, and the like. Infections diseases may also be caused by bacteria including Bacillus antracis, Borrelia burgdorferi, Campylobacter jejuni, Chlamydia trachomatis, Clostridium botulinum, Clostridium tetani, Diptheria, E. coli, Legionella, Helicobacter pylori, Mycobacterium rickettsia, Mycoplasma nesisseria, Pertussis, Pseudomonas aeruginosa, S. pneumonia, Streptococcus, Staphylococcus, Vibria cholerae, Yersinia pestis, and the like. Infectious diseases may also be caused by fungi such as Aspergillus fumigatus, Blastomyces dermatitidis, Candida albicans, Coccidioides immitis, Cryptococcus neoformans, Histoplasma capsulatum, Penicillium marneffei, and the like. Infectious diseases may also be caused by protozoa and parasites such as chlamydia, kokzidioa, leishmania, malaria, rickettsia, trypanosoma, and the like.

Furthermore, inventive multivalent antibody analogs disclosed herein may be used to prevent or treat additional conditions including but not limited to heart conditions such as congestive heart failure (CHF), myocarditis and other conditions of the myocardium; skin conditions such as rosecea, acne, and eczema; bone and tooth conditions such as bone loss, osteoporosis, Paget's disease, Langerhans' cell histiocytosis, periodontal disease, disuse osteopenia, osteomalacia, monostotic fibrous dysplasia, polyostotic fibrous dysplasia, bone metastasis, bone pain management, humoral malignant hypercalcemia, periodontal reconstruction, spinal cord injury, and bone fractures; metabolic conditions such as Gaucher's disease; endocrine conditions such as Cushing's syndrome; and neurological and neurodegenerative conditions such as Alzheimer's disease.

Pharmaceutical compositions are contemplated wherein an inventive multivalent antibody analog disclosed herein and one or more therapeutically active agents are formulated. Formulations of the inventive multivalent antibody analogs disclosed herein are prepared for storage by mixing said inventive multivalent antibody analog having the desired degree of purity with optional pharmaceutically acceptable carriers, excipients or stabilizers (Remington's Pharmaceutical Sciences 16th edition, Osol, A. Ed., 1980), in the form of lyophilized formulations or aqueous solutions. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONIC S™ or polyethylene glycol (PEG). In one embodiment, the pharmaceutical composition that comprises the inventive multivalent antibody analog disclosed herein may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Some embodiments include at least one of the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration may be sterile. This is readily accomplished by filtration through sterile filtration membranes or other methods.

The inventive multivalent antibody analogs disclosed herein may also be formulated as immunoliposomes. A liposome is a small vesicle comprising various types of lipids, phospholipids and/or surfactant that is useful for delivery of a therapeutic agent to a mammal. Liposomes containing the inventive multivalent antibody analog are prepared by methods known in the art. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

An inventive multivalent antibody analog and other therapeutically active agents may also be entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980. Sustained-release preparations may be prepared. Suitable examples of sustained-release preparations include semipermeable matrices of solid hydrophobic polymer, which matrices are in the form of shaped articles, e.g. films, or microcapsules. Examples of sustained-release matrices include polyesters, hydrogels (for example poly(2-hydroxyethyl-methacrylate), or poly(vinylalcohol)), polylactides, copolymers of L-glutamic acid and gamma ethyl-L-glutamate, non-degradable ethylene-vinyl acetate, degradable lactic acid-glycolic acid copolymers such as the Lupron Depot® (which are injectable microspheres composed of lactic acid-glycolic acid copolymer and leuprolide acetate), poly-D-(−)-3-hydroxybutyric acid, and ProLease® (commercially available from Alkermes), which is a microsphere-based delivery system composed of the desired bioactive molecule incorporated into a matrix of poly-DL-lactide-co-glycolide (PLG). Administration of the pharmaceutical composition comprising an inventive multivalent antibody analog disclosed herein, e.g., in the form of a sterile aqueous solution, may be done in a variety of ways, including, but not limited to orally, subcutaneously, intravenously, intranasally, intraotically, transdermally, topically (e.g., gels, salves, lotions, creams, etc.), intraperitoneally, intramuscularly, intrapulmonary, vaginally, parenterally, rectally, or intraocularly. In some instances, for example for the treatment of wounds, inflammation, etc., the inventive multivalent antibody analog may be directly applied as a solution or spray. As is known in the art, the pharmaceutical composition may be formulated accordingly depending upon the manner of introduction. Subcutaneous administration may be used in circumstances where the patient may self-administer the pharmaceutical composition. Many protein therapeutics are not sufficiently potent to allow for formulation of a therapeutically effective dose in the maximum acceptable volume for subcutaneous administration. This problem may be addressed in part by the use of protein formulations comprising arginine-HCl, histidine, and polysorbate. Inventive multivalent antibody analogs disclosed herein may be more amenable to subcutaneous administration due to, for example, increased potency, improved serum half-life, or enhanced solubility. As is known in the art, protein therapeutics are often delivered by IV infusion or bolus. The inventive multivalent antibody analogs disclosed herein may also be delivered using such methods. For example, administration may be by intravenous infusion with 0.9% sodium chloride as an infusion vehicle.

Pulmonary delivery may be accomplished using an inhaler or nebulizer and a formulation comprising an aerosolizing agent. For example, AERx® inhalable technology commercially available from Aradigm, or Inhance™ pulmonary delivery system commercially available from Nektar Therapeutics may be used. Furthermore, inventive multivalent antibody analogs disclosed herein may be amenable to oral delivery.

In addition, any of a number of delivery systems are known in the art and may be used to administer the inventive multivalent antibody analogs disclosed herein. Examples include, but are not limited to, encapsulation in liposomes, microparticles, microspheres (e.g., PLA/PGA microspheres), and the like. Alternatively, an implant of a porous, non-porous, or gelatinous material, including membranes or fibers, may be used. Sustained release systems may comprise a polymeric material or matrix such as polyesters, hydrogels, poly(vinylalcohol), polylactides, copolymers of L-glutamic acid and ethyl-L-gutamate, ethylene-vinyl acetate, lactic acid-glycolic acid copolymers such as the Lupron Depot®, and poly-D-(−)-3-hydroxyburyric acid. It is also possible to administer a nucleic acid encoding an inventive multivalent antibody analog disclosed herein, for example by retroviral infection, direct injection, or coating with lipids, cell surface receptors, or other transfection agents. In all cases, controlled release systems may be used to release the inventive multivalent antibody analog at or close to the desired location of action.

The dosing amounts and frequencies of administration are, in one embodiment, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art. The concentration of the therapeutically active inventive multivalent antibody analog in the formulation may vary from about 0.1 to 100 weight %. In one embodiment, the concentration of the inventive multivalent antibody analog is in the range of 0.003 to 1.0 molar. In order to treat a patient, a therapeutically effective dose of the inventive multivalent antibody analog disclosed herein may be administered. By “therapeutically effective dose” herein is meant a dose that produces the effects for which it is administered. The exact dose will depend on the purpose of the treatment, and will be ascertainable by one skilled in the art using known techniques. Dosages may range from 0.0001 to 100 mg/kg of body weight or greater, for example 0.1, 1, 10, or 50 mg/kg of body weight. In one embodiment, dosages range from 1 to 10 mg/kg. In some embodiments, only a single dose of the inventive multivalent antibody analogsis used. In other embodiments, multiple doses of the inventive multivalent antibody analog are administered. The elapsed time between administrations may be less than 1 hour, about 1 hour, about 1-2 hours, about 2-3 hours, about 3-4 hours, about 6 hours, about 12 hours, about 24 hours, about 48 hours, about 2-4 days, about 4-6 days, about 1 week, about 2 weeks, or more than 2 weeks. In other embodiments the inventive multivalent antibody analogs disclosed herein are administered in metronomic dosing regimes, either by continuous infusion or frequent administration without extended rest periods. Such metronomic administration may involve dosing at constant intervals without rest periods. Typically such regimens encompass chronic low-dose or continuous infusion for an extended period of time, for example 1-2 days, 1-2 weeks, 1-2 months, or up to 6 months or more. The use of lower doses may minimize side effects and the need for rest periods.

In certain embodiments the inventive multivalent antibody analogs disclosed herein and one or more other prophylactic or therapeutic agents are cyclically administered to the patient. Cycling therapy involves administration of a first agent at one time, a second agent at a second time, optionally additional agents at additional times, optionally a rest period, and then repeating this sequence of administration one or more times. The number of cycles is typically from 2-10. Cycling therapy may reduce the development of resistance to one or more agents, may minimize side effects, or may improve treatment efficacy.

The inventive multivalent antibody analogs disclosed herein may be administered concomitantly with one or more other therapeutic regimens or agents. The additional therapeutic regimes or agents may be used to improve the efficacy or safety of the inventive multivalent antibody analog. Also, the additional therapeutic regimes or agents may be used to treat the same disease or a comorbidity rather than to alter the action of the inventive multivalent antibody analog. For example, an inventive multivalent antibody analog disclosed herein may be administered to the patient along with chemotherapy, radiation therapy, or both chemotherapy and radiation therapy.

The terms “in combination with” and “co-administration” are not limited to the administration of said prophylactic or therapeutic agents at exactly the same time. Instead, it is meant that the inventive multivalent antibody analog disclosed herein and the other agent or agents are administered in a sequence and within a time interval such that they may act together to provide a benefit that is increased versus treatment with only either the inventive multivalent antibody analog disclosed herein or the other agent or agents. In some embodiments, inventive multivalent antibody analogs disclosed herein and the other agent or agents act additively, and sometimes synergistically. Such molecules are suitably present in combination in amounts that are effective for the purpose intended. The skilled medical practitioner can determine empirically, or by considering the pharmacokinetics and modes of action of the agents, the appropriate dose or doses of each therapeutic agent, as well as the appropriate timings and methods of administration. The inventive multivalent antibody analogs disclosed herein may be administered in combination with one or more other prophylactic or therapeutic agents, including but not limited to cytotoxic agents, chemotherapeutic agents, antibiotics, antifungal agents, antiviral agents, cytokines, growth inhibitory agents, anti-hormonal agents, kinase inhibitors, anti-angiogenic agents, cardioprotectants, immunostimulatory agents, immunosuppressive agents, agents that promote proliferation of hematological cells, angiogenesis inhibitors, protein tyrosine kinase (PTK) inhibitors, other antibodies, Fc fusions, or immunoglobulins, or other therapeutic agents. The therapies of the invention may be combined with other immunotherapies. The therapies of the invention may be combined with antagonists of chemokines or cytokines, including but not limited to antibodies and Fc fusions. The inventive multivalent antibody analogs disclosed herein may be combined with other therapeutic regimens. For example, in one embodiment, the patient to be treated with an inventive multivalent antibody analog disclosed herein may also receive radiation therapy. Radiation therapy can be administered according to protocols commonly employed in the art and known to the skilled artisan. Such therapy includes but is not limited to cesium, iridium, iodine, or cobalt radiation. The radiation therapy may be whole body irradiation, or may be directed locally to a specific site or tissue in or on the body, such as the lung, bladder, or prostate. Optionally, the radiation therapy may be administered as a single dose or as multiple, sequential doses. The skilled medical practitioner can determine empirically the appropriate dose or doses of radiation therapy useful herein. In accordance with another, an inventive multivalent antibody analog disclosed herein and one or more other anti-cancer therapies are employed to treat cancer cells ex vivo. It is contemplated that such ex vivo treatment may be useful in bone marrow transplantation and particularly, autologous bone marrow transplantation. For instance, treatment of cells or tissue(s) containing cancer cells with an inventive multivalent antibody analog and one or more other anti-cancer therapies, such as described above, can be employed to deplete or substantially deplete the cancer cells prior to transplantation in a recipient patient. It is of course contemplated that the inventive multivalent antibody analogs disclosed herein may employ in combination with still other therapeutic techniques such as surgery.

Additional exemplary, non-limiting embodiments of the invention are set forth below:

### Embodiment 1

A multivalent antibody analog comprising a first polypeptide and a second polypeptide, wherein:

a) the first polypeptide comprises a first heavy chain comprising a variable heavy region, a CH2 domain or a variant thereof, and a CH3 domain or a variant thereof; wherein the C-terminus of the CH3 domain or variant thereof is covalently attached to a first variable light domain (V_{L}); and

b) the second polypeptide comprises a first light chain covalently attached to the N-terminus of an Fc region of a heavy chain, wherein the Fc region comprises a CH2 domain or a variant thereof and a CH3 domain or a variant thereof, and wherein the CH3 domain or variant thereof is covalently attached to a first variable heavy domain (V_{H});

wherein said first heavy chain and said first light chain form a first antigen binding site and said first V_{L} and said first V_{H} form a second antigen binding site.

Embodiment 2. The multivalent antibody analog according to Embodiment 1, wherein the first polypeptide and the second polypeptide each further comprises a hinge region, and wherein said hinge regions each contain at least one thiol group that is capable of participating in an intermolecular disulfide bond such that the first and the second polypeptide are covalently linked as a result of formation of the disulfide bond.

### Embodiment 3

The multivalent antibody analog according to Embodiment 2, wherein the thiol group is provided by a cysteine residue.

### Embodiment 4

The multivalent antibody analog according to any one of Embodiments 1 through 3, wherein the first VL is covalently attached to the CH3 domain, or variant thereof, of the first heavy chain via a linker moiety.

### Embodiment 5

The multivalent antibody analog according to any one of Embodiments 1 through 4, wherein the first VH is covalently attached to the CH3 domain, or variant thereof, of the Fc region via a linker moiety.

### Embodiment 6

The multivalent antibody analog according to any one of Embodiments 1 through 5, wherein the antibody analog further comprises a third antigen binding site.

### Embodiment 7

The multivalent antibody analog according to Embodiment 6, wherein the third antigen binding site is covalently attached via a linker moiety to either:

the first VL; or

the first VH.

### Embodiment 8

The multivalent antibody analog according to Embodiment 6 or Embodiment 7, wherein the third antigen binding site comprises a single chain variable region (scFv), wherein said scFv comprises a second VL that is covalently attached to a second VH.

### Embodiment 9

The multivalent antibody analog according to Embodiment 8, wherein the second VL is covalently attached to the second VH via a linker moiety.

### Embodiment 10

The multivalent antibody analog according to Embodiment 8 or Embodiment 9, wherein:

the second VL is attached to the first VL via a linker moiety;

the second VH is attached to the first VH via a linker moiety;

the second VL is attached to the first VH via a linker moiety; or

the second VH is attached to the first VL via the third linker moiety.

### Embodiment 11

The multivalent antibody analog according to any one of Embodiments 6 through 10, wherein the antibody analog further comprises a fourth antigen binding site.

### Embodiment 12

The multivalent antibody analog according to Embodiment 11, wherein the fourth antigen binding site is covalently attached either:

the first VL via a linker moiety; or

the first VH via a linker moiety.

### Embodiment 13

The multivalent antibody analog according to Embodiment 11 or Embodiment 12, wherein the fourth antigen binding site comprises a second single chain variable region (scFv), wherein said second scFv comprises a third VL that is covalently attached to a third VH.

### Embodiment 14

The multivalent antibody analog according to Embodiment 13, wherein the third VL is covalently attached to the third VH via a linker moiety.

### Embodiment 15

The multivalent antibody analog according to Embodiment 13 or Embodiment 14, wherein:

the third VL is attached to the first VL via a linker moiety;

the third VH is attached to the first VH via a linker moiety;

the second VL is attached to the first VH via a linker moiety; or

the second VH is attached to the first VL via a linker moiety.

### Embodiment 16

The multivalent antibody analog according to any one of Embodiments 4 through 15, wherein one or more of the linker moieties independently comprises a peptide from 1 to 75 amino acids in length, inclusive.

### Embodiment 17

The multivalent antibody analog according to any one of Embodiments 4 through 16, wherein one or more of the linker moieties independently comprises at least one of the 20 naturally occurring amino acids.

### Embodiment 18

The multivalent antibody analog according to any one of Embodiments 4 through 17, wherein the one or more of the linker moieties independently comprises at least one non-natural amino acid incorporated by chemical synthesis, post-translational chemical modification or by in vivo incorporation by recombinant expression in a host cell.

### Embodiment 19

The multivalent antibody analog according to any one of Embodiments 4 through 18, wherein the one or more of the linker moieties independently comprises one or more amino acids selected from the group consisting of serine, glycine, alanine, proline, asparagine, glutamine, glutamate, aspartate, and lysine.

### Embodiment 20

The multivalent antibody analog according to any one of Embodiments 4 through 19, wherein the one or more of the linker moieties independently comprises a majority of amino acids that are sterically unhindered.

### Embodiment 21

The multivalent antibody analog according to any one of Embodiments 4 through 20, wherein the one or more of the linker moieties independently comprises one or more of the following: an acidic linker, a basic linker, and a structural motif.

### Embodiment 22

The multivalent antibody analog according to any one of Embodiments 4 through 21, wherein one or more of the linker moieties independently comprises: polyglycine, polyalanine, poly(Gly-Ala), or poly(Gly-Ser).

### Embodiment 23

The multivalent antibody analog according to any one of Embodiments 4 through 22, wherein one or more of the linker moieties independently comprises: a polyglycine selected from the group consisting of: (Gly)₃, (Gly)₄ (SEQ ID NO: 1), and (Gly)₅ (SEQ ID NO: 2).

### Embodiment 24

The multivalent antibody analog according to any one of Embodiments 4 through 23 wherein one or more of the linker moieties independently comprises (Gly)₃Lys(Gly)₄ (SEQ ID NO: 3); (Gly)₃AsnGlySer(Gly)₂ (SEQ ID NO: 4); (Gly)₃Cys(Gly)₄ (SEQ ID NO: 5); and GlyProAsnGlyGly (SEQ ID NO: 24).

### Embodiment 25

The multivalent antibody analog according to any one of Embodiments 4 through 24, wherein one or more of the linker moieties independently comprises a combination of Gly and Ala.

### Embodiment 26

The multivalent antibody analog according to any one of Embodiments 4 through 25, wherein one or more of the linker moieties independently comprises a combination of Gly and Ser.

### Embodiment 27

The multivalent antibody analog according to any one of Embodiments 4 through 26, wherein one or more of the linker moieties independently comprises a combination of:

Gly and Glu; or

Gly and Asp.

### Embodiment 28

The multivalent antibody analog according to any one of Embodiments 4 through 27, wherein one or more of the linker moieties independently comprises a combination of Gly and Lys.

### Embodiment 29

The multivalent antibody analog according to any one of Embodiments 4 through 28, wherein one or more of the linker moieties independently comprises a sequence selected from group consisting of: [Gly-Ser]ₙ (SEQ ID NO: 6); [Gly-Gly-Ser]ₙ (SEQ ID NO: 7); [Gly-Gly-Gly-Ser]ₙ (SEQ ID NO: 8); [Gly-Gly-Gly-Gly-Ser]ₙ (SEQ ID NO: 9); [Gly-Gly-Gly-Gly-Ser-Gly-Gly-Gly-Gly]ₙ (SEQ ID NO: 10); [Gly-Gly-Gly-Gly-Ser-Gly-Gly-Gly-Gly-Ser-Gly-Gly-Gly-Gly]ₙ (SEQ ID NO: 11); [Gly-Gly-Gly-Gly-Ser Gly-Gly-Gly-Gly-Ser-Gly-Gly-Gly-Gly-Ser-Gly-Gly-Gly-Gly]ₙ (SEQ ID NO: 12); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 13); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 14); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 15); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

Embodiment 30. The multivalent antibody analog according to any one of Embodiments 4 through 29, wherein one or more of the linker moieties independently comprises a sequence selected from the group consisting of: [Gly-Glu]ₙ (SEQ ID NO: 16); [Gly-Gly-Glu]ₙ (SEQ ID NO: 17); [Gly-Gly-Gly-Glu]ₙ (SEQ ID NO: 18); [Gly-Gly-Gly-Gly-Glu]ₙ (SEQ ID NO: 19); [Gly-Asp]ₙ (SEQ ID NO: 20); [Gly-Gly-Asp]ₙ (SEQ ID NO: 21); [Gly-Gly-Gly-Asp]ₙ (SEQ ID NO: 22); [Gly-Gly-Gly-Gly-Asp]ₙ (SEQ ID NO: 23); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

### Embodiment 31

The multivalent antibody analog according to any one of Embodiments 1 through 30, wherein the CH2 domain variant and the CH3 domain variant each independently comprises at least one different amino acid substitution such that a heterodimeric domain pair is generated such that heterodimerization of said variants is favored over homodimerization.

### Embodiment 32

The multivalent antibody analog according to any one of Embodiments 1 through 31, wherein either:

a) the CH2 domain variant and the CH3 domain variant each independently comprises a at least one protuberance in either the CH2 domain or the CH3 domain of the first polypeptide and at least one corresponding cavity in the CH2 domain or the CH3 domain of the second; or

b) the CH2 domain variant and the CH3 domain variant each independently comprises at least one cavity in either the CH2 domain or the CH3 domain of the first polypeptide and at least one corresponding protuberance in the CH2 domain or the CH3 domain of the second polypeptide.

### Embodiment 33

The multivalent antibody analog according to any one of Embodiments 1 through 32, wherein either:

a) the CH2 domain variant and the CH3 domain variant each independently comprises at least one substituted negatively-charged amino acid in either the CH2 domain or the CH3 domain of the first polypeptide and at least one corresponding positively-charged amino acid in either the CH2 domain or the CH3 domain of the second polypeptide; or

b) the CH2 domain variant and the CH3 domain variant each independently comprises at least one substituted positively-charged amino acid in either the CH2 domain or the CH3 domain of the first polypeptide and at least one corresponding substituted negatively-charged substituted amino acid in either the CH2 domain or the CH3 domain of the second polypeptide.

### Embodiment 34

A multivalent antibody analog comprising a first polypeptide and a second polypeptide, wherein:

a) the first polypeptide comprises a first heavy chain comprising a variable heavy region, a CH2 domain or a variant thereof, and a CH3 domain or a variant thereof;

b) the second polypeptide comprises a first light chain covalently attached to the N-terminus of an Fc region of a heavy chain, wherein the Fc region comprises a CH2 domain or a variant thereof and a CH3 domain or a variant thereof; and

c) either the first polypeptide or the second polypeptide further comprises a single chain variable region (scFv) comprising a first VL that is covalently attached to a first VH, wherein said scFv is covalently attached to the CH3 domain or variant thereof of said first polypeptide or said second polypeptide;

wherein said first heavy chain and said first light chain form a first antigen binding site and the first VL and the first VH form a second antigen binding site.

### Embodiment 35

A multivalent antibody analog comprising a first polypeptide and a second polypeptide, wherein:

a) the first polypeptide comprises a first heavy chain comprising a variable heavy region, a CH2 domain or a variant thereof, and a CH3 domain or a variant thereof;

b) the second polypeptide comprises a first light chain covalently attached to the N-terminus of an Fc region of a heavy chain, wherein the Fc region comprises a CH2 domain or a variant thereof and a CH3 domain or a variant thereof; and

c) the first polypeptide further comprises a single chain variable region (scFv) comprising a first VL that is covalently attached to a first VH; and the second polypeptide further comprises a single chain variable region (scFv) comprising a second VL that is covalently attached to a second VH; wherein one scFv is covalently attached to the CH3 domain of variant thereof of said first polypeptide and the other scFv is covalently attached to the CH3 domain or variant thereof of said second polypeptide;

wherein said first heavy chain and said first light chain form a first antigen binding site, the first VL and the first VH form a second antigen binding site, and the second VL and the second VH form a third antigen binding site.

### Embodiment 36

The multivalent antibody analog according to Embodiment 34 or Embodiment 35, wherein the first polypeptide and the second polypeptide each further comprises a hinge region, and wherein said hinge regions each contain at least one thiol group that is capable of participating in an intermolecular disulfide bond such that the first and the second polypeptides are covalently linked as a result of formation of the disulfide bond.

### Embodiment 37

The multivalent antibody analog according to Embodiment 36, wherein the thiol group is provided by a cysteine residue.

### Embodiment 38

The multivalent antibody analog according to any one of Embodiments 34 through 37, wherein:

the first VL is attached to the CH3 domain, or variant thereof, of the first heavy chain via a linker moiety;

the first VL is attached to the CH3 domain, or variant thereof, of the Fc region via a linker moiety;

the first VH is attached to the CH3 domain, or variant thereof, of the first heavy chain via a linker moiety; or

the first VH is attached to the CH3 domain, or variant thereof, of the Fc region via a linker moiety.

### Embodiment 39

The multivalent antibody analog according to any one of Embodiments 35 through 38, wherein:

the second VL is attached to the CH3 domain, or variant thereof, of the first heavy chain via a linker moiety;

the second VL is attached to the CH3 domain, or variant thereof, of the Fc region via a linker moiety;

the second VH is attached to the CH3 domain, or variant thereof, of the first heavy chain via a linker moiety; or

the second VH is attached to the CH3 domain, or variant thereof, of the Fc region via a linker moiety.

### Embodiment 40

The multivalent antibody analog according to any one of Embodiments 38 through 39, wherein one or more of the linker moieties independently comprises a peptide from 1 to 75 amino acids in length, inclusive.

### Embodiment 41

The multivalent antibody analog according to any one of Embodiments 38 through 40, wherein one or more of the linker moieties independently comprises at least one of the 20 naturally occurring amino acids.

### Embodiment 42

The multivalent antibody analog according to any one of Embodiments 38 through 41, wherein the one or more of the linker moieties independently comprises at least one non-natural amino acid incorporated by chemical synthesis, post-translational chemical modification or by in vivo incorporation by recombinant expression in a host cell.

### Embodiment 43

The multivalent antibody analog according to any one of Embodiments 38 through 42, wherein the one or more of the linker moieties independently comprises one or more amino acids selected from the group consisting of serine, glycine, alanine, proline, asparagine, glutamine, glutamate, aspartate, and lysine.

### Embodiment 44

The multivalent antibody analog according to any one of Embodiments 38 through 43, wherein the one or more of the linker moieties independently comprises a majority of amino acids that are sterically unhindered.

### Embodiment 45

The multivalent antibody analog according to any one of Embodiments 38 through 44, wherein the one or more of the linker moieties independently comprises one or more of the following: an acidic linker, a basic linker, and a structural motif.

### Embodiment 46

The multivalent antibody analog according to any one of Embodiments 38 through 45, wherein one or more of the linker moieties independently comprises: polyglycine, polyalanine, poly(Gly-Ala), or poly(Gly-Ser).

### Embodiment 47

The multivalent antibody analog according to any one of Embodiments 38 through 46, wherein one or more of the linker moieties independently comprises: a polyglycine selected from the group consisting of: (Gly)3, (Gly)4 (SEQ ID NO: 1), and (Gly)5 (SEQ ID NO: 2).

### Embodiment 48

The multivalent antibody analog according to any one of Embodiments 38 through 47 wherein one or more of the linker moieties independently comprises (Gly)₃Lys(Gly)₄ (SEQ ID NO: 3); (Gly)₃AsnGlySer(Gly)₂ (SEQ ID NO: 4); (Gly)₃Cys(Gly)₄ (SEQ ID NO: 5); and GlyProAsnGlyGly (SEQ ID NO: 24).

### Embodiment 49

The multivalent antibody analog according to any one of Embodiments 38 through 48, wherein one or more of the linker moieties independently comprises a combination of Gly and Ala.

### Embodiment 50

The multivalent antibody analog according to any one of Embodiments 38 through 49, wherein one or more of the linker moieties independently comprises a combination of Gly and Ser.

### Embodiment 51

The multivalent antibody analog according to any one of Embodiments 38 through 50, wherein one or more of the linker moieties independently comprises a combination of:

Gly and Glu; or

Gly and Asp.

### Embodiment 52

The multivalent antibody analog according to any one of Embodiments 38 through 51, wherein one or more of the linker moieties independently comprises a combination of Gly and Lys.

### Embodiment 53

The multivalent antibody analog according to any one of Embodiments 38 through 52, wherein one or more of the linker moieties independently comprises a sequence selected from group consisting of: [Gly-Ser]ₙ (SEQ ID NO: 6); [Gly-Gly-Ser]ₙ (SEQ ID NO: 7); [Gly-Gly-Gly-Ser]ₙ (SEQ ID NO: 8); [Gly-Gly-Gly-Gly-Ser]ₙ (SEQ ID NO: 9); [Gly-Gly-Gly-Gly-Ser-Gly-Gly-Gly-Gly]ₙ (SEQ ID NO: 10); [Gly-Gly-Gly-Gly-Ser-Gly-Gly-Gly-Gly-Ser-Gly-Gly-Gly-Gly]ₙ (SEQ ID NO: 11); [Gly-Gly-Gly-Gly-Ser Gly-Gly-Gly-Gly-Ser-Gly-Gly-Gly-Gly-Ser-Gly-Gly-Gly-Gly]ₙ (SEQ ID NO: 12); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 13); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 14); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 15); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

### Embodiment 54

The multivalent antibody analog according to any one of Embodiments 38 through 53, wherein one or more of the linker moieties independently comprises a sequence selected from the group consisting of: [Gly-Glu]ₙ (SEQ ID NO: 16); [Gly-Gly-Glu]ₙ (SEQ ID NO: 17); [Gly-Gly-Gly-Glu]ₙ (SEQ ID NO: 18); [Gly-Gly-Gly-Gly-Glu]ₙ (SEQ ID NO: 19); [Gly-Asp]ₙ (SEQ ID NO: 20); [Gly-Gly-Asp]ₙ (SEQ ID NO: 21); [Gly-Gly-Gly-Asp]ₙ (SEQ ID NO: 22); [Gly-Gly-Gly-Gly-Asp]ₙ (SEQ ID NO: 23); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

### Embodiment 55

The multivalent antibody analog according to any one of Embodiments 33 through 54, wherein the CH2 domain variant and the CH3 domain variant each independently comprises at least one different amino acid substitution such that a heterodimeric domain pair is generated such that heterodimerization of said variants is favored over homodimerization.

### Embodiment 56

The multivalent antibody analog according to any one of Embodiments 33 through 55, wherein either:

a) the CH2 domain variant and the CH3 domain variant each independently comprises a at least one protuberance in either the CH2 domain or the CH3 domain of the first polypeptide and at least one corresponding cavity in the CH2 domain or the CH3 domain of the second; or

b) the CH2 domain variant and the CH3 domain variant each independently comprises at least one cavity in either the CH2 domain or the CH3 domain of the first polypeptide and at least one corresponding protuberance in the CH2 domain or the CH3 domain of the second polypeptide.

### Embodiment 57

The multivalent antibody analog according to any one of Embodiments 33 through 56, wherein either:

a) the CH2 domain variant and the CH3 domain variant each independently comprises at least one substituted negatively-charged amino acid in either the CH2 domain or the CH3 domain of the first polypeptide and at least one corresponding positively-charged amino acid in either the CH2 domain or the CH3 domain of the second polypeptide; or

b) the CH2 domain variant and the CH3 domain variant each independently comprises at least one substituted positively-charged amino acid in either the CH2 domain or the CH3 domain of the first polypeptide and at least one corresponding substituted negatively-charged substituted amino acid in either the CH2 domain or the CH3 domain of the second polypeptide.

### Embodiment 58

The multivalent antibody analog according to any one of Embodiments 1 through 57, wherein at least one antigen binding site comprises at least one humanized variable heavy domain or at least one humanized variable light domain.

### Embodiment 59

The multivalent antibody analog according to any one of Embodiments 1 through 58, wherein at least one antigen binding site comprises at least one complimentary determining region CDR that is derived from a non-human antibody or antibody fragment.

### Embodiment 60

The multivalent antibody analog according to any one of Embodiments 4 through 33 and 40 through 59, wherein the length of each linker moiety is independently selected from 1 through 35 amino acids in length.

### Embodiment 61

The multivalent antibody analog according to any one of Embodiments 4 through 33 and 40 through 60, wherein the length of each linker moiety is independently selected from 5 through 35 amino acids in length.

### Embodiment 62

The multivalent antibody analog according to any one of Embodiments 4 through 33 and 40 through 61, wherein the length of each linker moiety is independently selected from 10 through 35 amino acids in length.

### Embodiment 63

The multivalent antibody analog according to any one of Embodiments 4 through 33 and 40 through 62, wherein the length of each linker moiety is independently selected from 14 through 35 amino acids in length.

### Embodiment 64

The multivalent antibody analog according to any one of Embodiments 4 through 33 and 40 through 63, wherein the length of each linker moiety is independently selected from 19 through 35 amino acids in length.

### Embodiment 65

The multivalent antibody analog according to any one of Embodiments 1 through 64, wherein at least one antigen binding site binds an epitope from a tumor associated antigen, a hormone receptor, a cytokine receptor, chemokine receptor, a growth factor receptor, an immune activating receptor, a hormone, a cytokine, a chemokine, a growth factor, a G protein-coupled receptor, or a transmembrane receptor.

### Embodiment 66

The multivalent antibody analog according to any one of Embodiments 1 through 65, wherein at least one antigen binding site binds a target associated with an autoimmune disorder, an inflammatory disorder, an oncological disorder, neuromuscular disorder, a neurodegenerative disorder, a metabolic disorder, or an infectious disease.

### Embodiment 67

The multivalent antibody analog according to any one of Embodiments 1 through 66, wherein the antibody analog binds at least two different targets.

### Embodiment 68

The multivalent antibody analog according to any one of Embodiments 1 through 67, wherein the antibody analog binds at least three different targets.

### Embodiment 69

The multivalent antibody analog according to any one of Embodiments 1 through 68, wherein the antibody analog binds at least four different targets.

### Embodiment 70

The multivalent antibody analog according to any one of Embodiments 1 through 69, wherein the antibody analog binds at least one target monovalently.

### Embodiment 71

The multivalent antibody analog according to any one of Embodiments 1 through 70, wherein the antibody analog binds at least two targets monovalently.

### Embodiment 72

The multivalent antibody analog according to any one of Embodiments 1 through 71, wherein the antibody analog binds at least three targets monovalently.

### Embodiment 73

The multivalent antibody analog according to any one of Embodiments 1 through 72, wherein the antibody analog binds at least four targets monovalently.

### Embodiment 74

The multivalent antibody analog according to any one of Embodiments 1 through 73, wherein the antibody analog wherein at least one of the antigen binding sites comprises or is derived from a non-human species.

### Embodiment 75

The multivalent antibody analog according to any one of Embodiments 1 through 74, wherein the antibody analog wherein at least one of the antigen binding sites comprises a humanized variable domain or a humanized CDR.

### Embodiment 76

The multivalent antibody analog according to any one of Embodiments 1 through 75, wherein the antibody analog is selected from the group consisting of the antibody analogs described in the Examples.

### Embodiment 77

The multivalent antibody analog according to any one of Embodiments 1 through 76, wherein:

a) the first polypeptide further comprises a CH1 domain or a variant thereof covalently attached to the CH2 domain or a variant thereof;

b) the first light chain of the second polypeptide comprises either a Vkappa domain or a Vlambda domain covalently attached to C-terminus of the VL domain and to the N-terminus of an Fc region of the heavy chain.

### Embodiment 78

A method of treating an autoimmune disorder, an inflammatory disorder, an oncological disorder, neuromuscular disorder, a neurodegenerative disorder, a metabolic disorder, or an infectious disease, the method comprising providing or administering a therapeutically effective amount of a multivalent antibody analog according to any one of Embodiments 1 through 77.

### Embodiment 79

A method of treating an autoimmune disorder, an inflammatory disorder, an oncological disorder, neuromuscular disorder, a neurodegenerative disorder, a metabolic disorder, or an infectious disease, the method comprising providing or administering a therapeutically effective amount of a multivalent antibody analog selected from the group consisting of the antibody analogs disclosed in the Examples.

### EXAMPLES

### Example 1

### Generation of Bispecific Antibody Analogs Comprising an N-Terminal Antibody Binding Region (Fab) x C-Terminal Antibody Variable Region (Fᵥ) that Binds to Target a Via the Fab and Target B Via the Fv

### Generation of a Exemplary Constructs Encoding Polypeptide 1: N-Terminal VH-CH1-Fc Region-C-Terminal VL Orientation

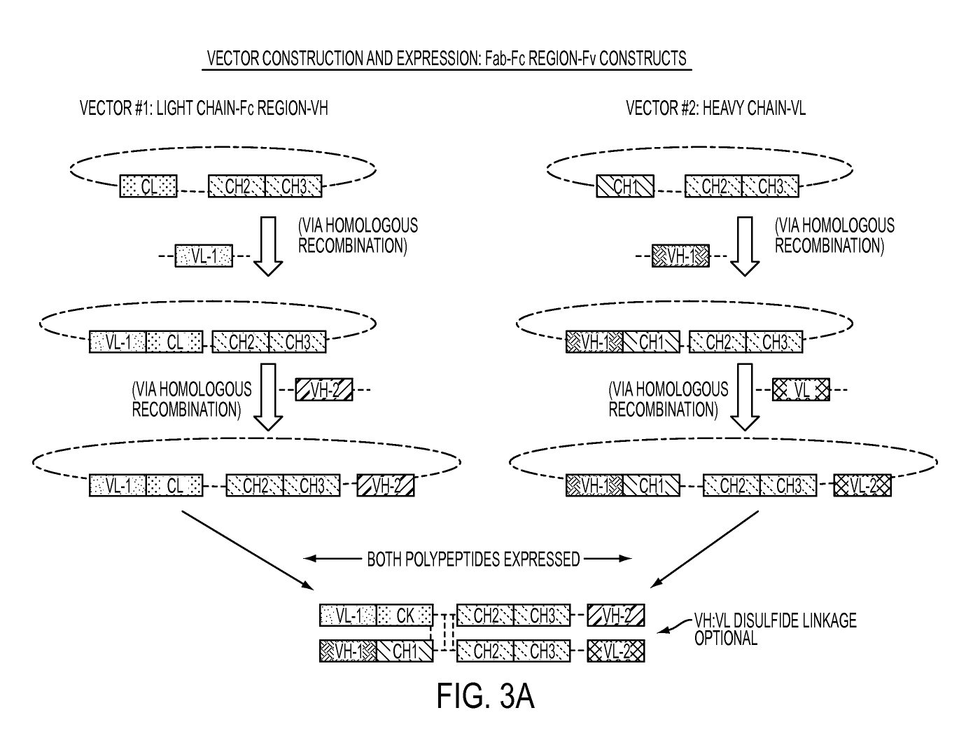

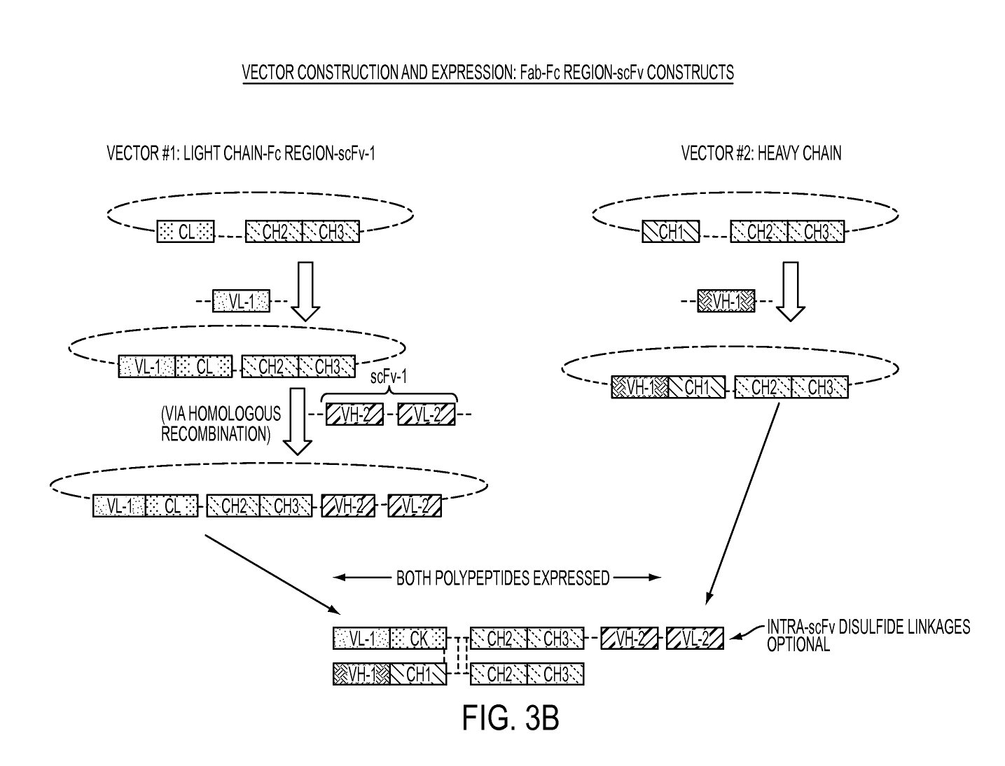

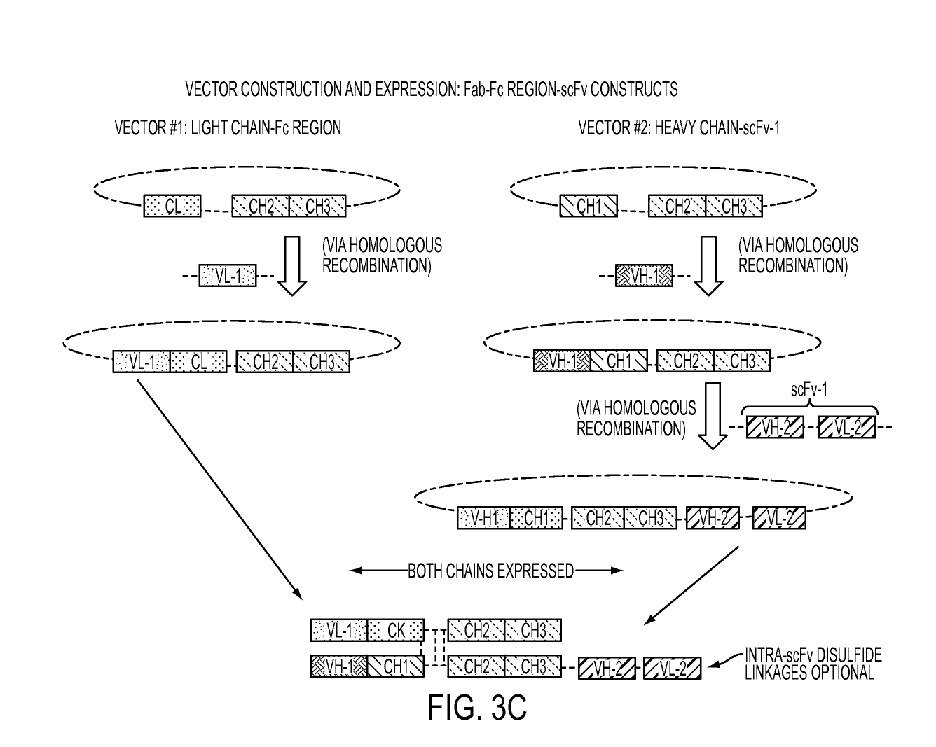

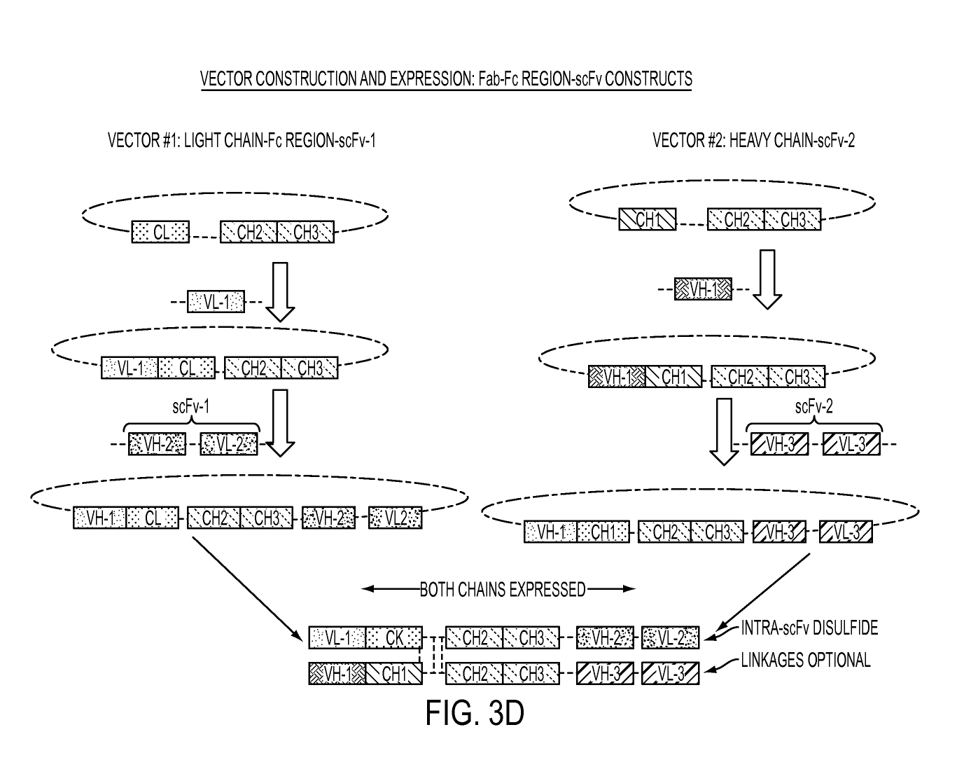

A vector harboring the coding sequence for CH1, hinge, CH2, and CH3 regions of human IgG (e.g., as represented in FIG. 3A, right side) contains a unique Sfil restriction site and allows for in-frame recombination/cloning N-terminal to the CH1 region, as well as a NotI restriction site that allows for in-frame recombination/cloning C-terminal to the CH3 region. A sample of this vector was first digested with the Sfil enzyme. VH-encoding nucleic acid of an IgG that binds Target A (“VH-1”) was amplified using primers that contain 5′ and 3′ flanking regions, respectively, that are complimentary to the Sfil restriction-digested ends of the linearized vector. Saccharomyces cerevesiae cells were then transformed with the amplified fragment and the Sfil-digested vector, allowed to recover for approximately one hour in media at 30 degrees Celsius, and then plated on appropriate plates. Colonies were then picked from the plates, grown up, and plasmid DNA extracted and the sequence confirmed to contain the VH-1, CH1, hinge, CH2, and CH3 regions as represented in FIG. 3A. A sample of the recovered, sequence-verified vector containing the VH-1, CH1, hinge, CH2, and CH3 regions was then digested with the NotI enzyme for transformation with each the VL-encoding nucleic acids described below.

In order to prepare VL-encoding nucleic acid that contains one of several different linkers, several different 5′ primers were prepared that were complimentary to an appropriate portion of the coding sequence for the VL region from an IgG that binds Target B (“VL-2”). Each of these primers also contained a sequence encoding one of the following linker sequences, immediately upstream and in-frame with the VL-coding sequence from the IgG that binds Target B (“VL-2):

Exemplary Linker 1.
(SEQ ID NO: 37)
Gly-Gly-Gly-Gly-Ser
Exemplary Linker 2.
(SEQ ID NO: 40)
Gly-Gly-Gly-Gly-Ser-Gly-Gly-Gly-Gly-Ser-Gly-Gly-
Gly-Gly
Exemplary Linker 3.
(SEQ ID NO: 41)
Gly-Gly-Gly-Gly-Ser-Gly-Gly-Gly-Gly-Ser-Gly-Gly-
Gly-Gly-Ser-Gly-Gly-Gly-Gly
Exemplary Linker 4.
(SEQ ID NO: 42)
Gly-Gly-Gly-Gly-Ser-Gly-Gly-Gly-Gly-Ser-Gly-Gly-
Gly-Gly-Ser-Gly-Gly-Gly-Gly-Ser-Gly-Gly-Gly-Gly
Exemplary Linker 5.
(SEQ ID NO: 43)
Gly-Gly-Gly-Gly-Ser-Gly-Gly-Gly-Gly-Ser-Gly-Gly-
Gly-Gly-Ser-Gly-Gly-Gly-Gly-Ser-Gly-Gly-Gly-Gly-
Ser-Gly-Gly-Gly-Gly
Exemplary Linker 6.
(SEQ ID NO: 44)
Gly-Gly-Gly-Gly-Ser-Gly-Gly-Gly-Gly-Ser-Gly-Gly-
Gly-Gly-Ser-Gly-Gly-Gly-Gly-Ser-Gly-Gly-Gly-Gly-
Ser-Gly-Gly-Gly-Gly-Ser-Gly-Gly-Gly-Gly

Each 5′ primer also contained nucleic acid that was complimentary to the NotI restriction-digested ends of the linearized vector at the 5′ end of the primer (i.e., upstream of the linker-encoding sequence). A 3′-primer was also prepared that contained, at its 3′ end, nucleic acid that encoded a six-histidine tag (SEQ ID NO: 38) (“His tag”), followed by a downstream sequence that was complimentary to the NotI restriction-digested ends of the linearized vector. The 3′ primer contained appropriate coding sequence upstream of the His tag that was complimentary to the coding sequence for the VL region from the IgG that binds Target B (“VL-2”).

The VL-encoding nucleic acid was then amplified using, in separate reactions, each 5′ primer (each one containing one of the linker-encoding sequences as described above) and the 3′ primer. Separate transformations were then performed in Saccharomyces cerevesiae cells with the each of VL-encoding nucleic acids described above (i.e., each nucleic acid encoding: the VL; and one of the linkers described above) and the Nod-digested vector. The transformed cells were then allowed to recover for approximately one hour in media at 30 degrees Celsius and then plated on appropriate plates. Colonies were then picked from the plates, grown up, and plasmid DNA extracted and the sequence confirmed to contain the VH-1, CH1, hinge, CH2, CH3, linker, and VL-2 regions as represented in FIG. 3A.

### Generation of a Exemplary Constructs Encoding Polypeptide 2: N-Terminal VL-CL-Fc Region-C-Terminal VH Orientation

A vector harboring the coding sequence for CL, hinge, CH2, and CH3 regions of human IgG (e.g., as represented in FIG. 3A, left side) contains a unique Sfil restriction site and allows for in-frame recombination/cloning N-terminal to the CL region, as well as a NotI restriction site that allows for in-frame recombination/cloning C-terminal to the CH3 region. A sample of this vector was first digested with the Sfil enzyme. VL-encoding nucleic acid of an IgG that binds Target A (“VL-1”) was amplified using primers that contain 5′ and 3′ flanking regions, respectively, that are complimentary to the Sfil restriction-digested ends of the linearized vector. Saccharomyces cerevesiae cells were then transformed with the amplified VL-encoding nucleic acid and the Sfil-digested vector, allowed to recover for approximately one hour in media at 30 degrees Celsius, and then plated on appropriate plates. Colonies were then picked from the plates, grown up, and plasmid DNA extracted and the sequence confirmed to contain the VL-1, CL, hinge, CH2, and CH3 regions as represented in FIG. 3A. A sample of the recovered, sequence-verified vector containing the VL-1, CL, hinge, CH2, and CH3 regions was then digested with the NotI enzyme for transformation with the VH-encoding nucleic acid described below.

In order to prepare VH-encoding nucleic acid that contains one of several different linkers, several different 5′ primers were prepared that were complimentary to an appropriate portion of the coding sequence for the VH region from an IgG that binds Target B (“VH-2”). Each of these primers also contained a sequence encoding one of the following linker sequences, immediately upstream and in-frame with the VH-coding sequence from the IgG that binds Target B (“VH-2):

Exemplary Linker 1.
(SEQ ID NO: 37)
Gly-Gly-Gly-Gly-Ser
Exemplary Linker 2.
(SEQ ID NO: 40)
Gly-Gly-Gly-Gly-Ser-Gly-Gly-Gly-Gly-Ser-Gly-Gly-
Gly-Gly
Exemplary Linker 3.
(SEQ ID NO: 41)
Gly-Gly-Gly-Gly-Ser-Gly-Gly-Gly-Gly-Ser-Gly-Gly-
Gly-Gly-Ser-Gly-Gly-Gly-Gly
Exemplary Linker 4.
(SEQ ID NO: 42)
Gly-Gly-Gly-Gly-Ser-Gly-Gly-Gly-Gly-Ser-Gly-Gly-
Gly-Gly-Ser-Gly-Gly-Gly-Gly-Ser-Gly-Gly-Gly-Gly
Exemplary Linker 5.
(SEQ ID NO: 43)
Gly-Gly-Gly-Gly-Ser-Gly-Gly-Gly-Gly-Ser-Gly-Gly-
Gly-Gly-Ser-Gly-Gly-Gly-Gly-Ser-Gly-Gly-Gly-Gly-
Ser-Gly-Gly-Gly-Gly
Exemplary Linker 6.
(SEQ ID NO: 44)
Gly-Gly-Gly-Gly-Ser-Gly-Gly-Gly-Gly-Ser-Gly-Gly-
Gly-Gly-Ser-Gly-Gly-Gly-Gly-Ser-Gly-Gly-Gly-Gly-
Ser-Gly-Gly-Gly-Gly-Ser-Gly-Gly-Gly-Gly.

Each 5′ primer also contained nucleic acid that was complimentary to the NotI restriction-digested ends of the linearized vector at the 5′ end of the primer (i.e., upstream of the linker-encoding sequence). A 3′-primer was also prepared that contained, at its 3′ end, nucleic acid that encoded a FLAG tag, followed by a downstream sequence that was complimentary to the NotI restriction-digested ends of the linearized vector. The 3′ primer also contained appropriate coding sequence upstream of the FLAG tag that was complimentary to the coding sequence for the VH region from the IgG that binds Target B (“VH-2”).

The VH-encoding nucleic acid was then amplified using, in separate reactions, each 5′ primer (each one containing one of the linker-encoding sequences as described above) and the 3′ primer. Separate transformations were then performed in Saccharomyces cerevesiae cells with the each of VH-encoding nucleic acids described above (i.e., each nucleic acid encoding: the VH; and one of the linkers described above) and the Nod-digested vector. The transformed cells were then allowed to recover for approximately one hour in media at 30 degrees Celsius and then plated on appropriate plates. Colonies were then picked from the plates, grown up, and plasmid DNA extracted and the sequence confirmed to contain the VH-1, CH1, hinge, CH2, CH3, linker, and VL-2 regions as represented in FIG. 3A.

### Expression of (Fab) x C-Terminal Antibody Variable Region (Fᵥ) Bispecific Antibody Analogs

Separate transformations were performed in Saccharomyces cerevesiae cells vectors encoding polypeptide 1 and one of polypeptide 2, prepared as described above, and the bispecific antibody analogs expressed thereby. A series of vectors encoding polypeptides 1 and 2 were also prepared as described above in which the N-terminal Fab portion of the bispecific analogs expressed thereby bind Target B, and the C-terminal Fv portions bind Target A.

### Purification of (Fab) x C-Terminal Antibody Variable Region (Fᵥ) Bispecific Antibody Analogs

A two-step purification scheme, provided below, was employed in order to purify each of the antibody analogs described above.

### Protein A Purification:

Protein A resin (MabSelect SuRe (GE Healthcare) was equilibrated in phosphate buffered saline, pH 7.4. Sample containing the bispecific antibody analog was applied to the column. The column was washed several times with wash buffer after addition of the sample containing the bispecific antibody analog to the column. The bispecific antibody analog was then eluted from the column by the addition of elution buffer (200 mM Acetic Acid, pH 2.0). Once eluted, the sample was neutralized with 2 M HEPES, pH 8.0.

### HisTag Purification:

HisTag resin (Ni Sepharose 6 Fast Flow, (GE Healthcare) was employed as the second purification step for each bispecific antibody analog. The HisTag resin was equilibrated in wash buffer (20 mM NaH₂PO₄, 500 mM NaCl, 20 mM Imidazole, pH 7.4). Sample containing the bispecific antibody analog was applied to the column. After addition of the sample to the column, the column was washed several times with wash buffer. The bispecific antibody analog was then eluted from the column by the addition of elution buffer (20 mM NaH₂PO₄, 500 mM NaCl, 500 mM Imidazole, pH 7.4).

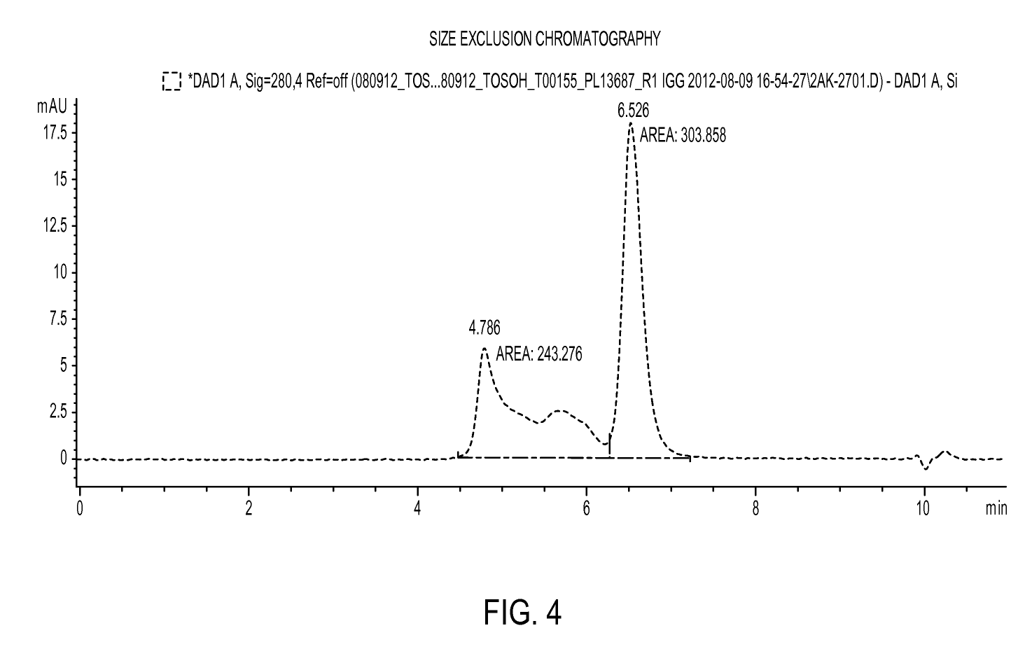

Expression and quality of each of the purified antibody analogs was assess by both reducing and non-reducing polyacrylamide gel electrophoresis (PAGE) and size exclusion chromatography (SEC) in accordance with methodologies known in the art. An Agilent 1100 HPLC was employed to monitor the column chromatography (TSKgel Super SW3000 column). The column was equilibrated with wash buffer (200 mM Sodium Phosphate, 250 mM Sodium Chloride pH 6.8) prior to use. Approximately 2-5 μg of protein sample was injected onto column and flow rate adjusted to 0.400 mL/min. Protein migration was monitored at wavelength 280 nm. Total assay time was approximately 11 min. The SEC profile of an exemplary bispecific antibody analog with the N-terminal antibody fragment (Fab) x C-terminal variable fragment (Fᵥ) is depicted in FIG. 4 in which the major peak, corresponding to the desired multivalent antibody analog, is evident.

### Binding and Affinity Measurements

This material was collected and used to carry out binding and affinity measurements using a Forte-Bio Octet Red 384 instrument generally in accordance with the manufacturer's instructions.

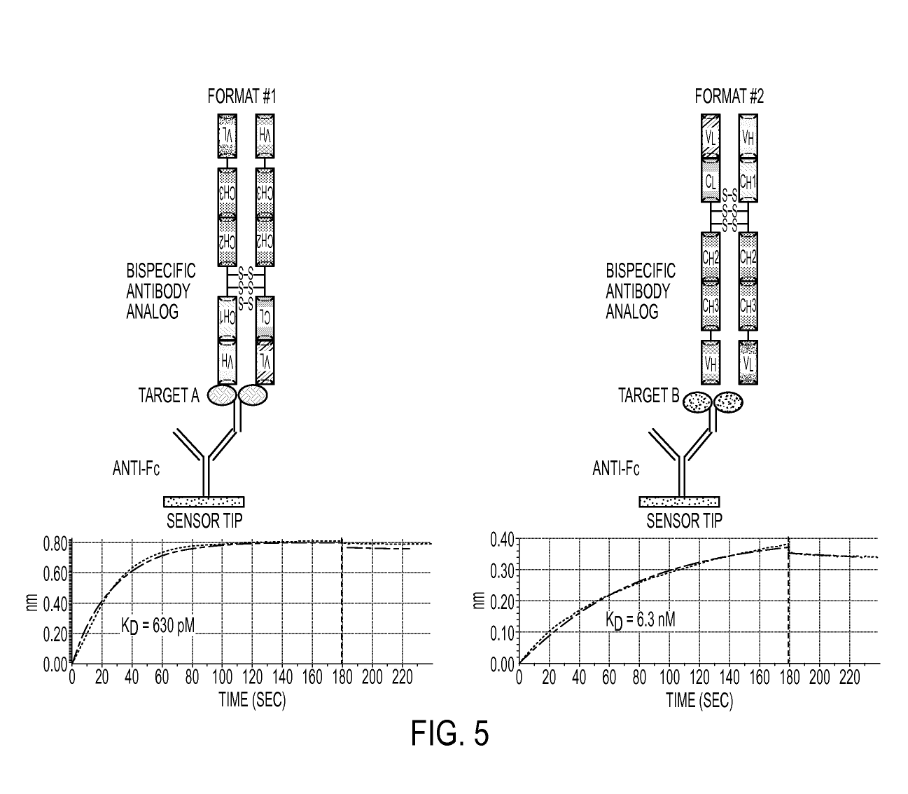

Monovalent binding and affinity measurements were first made using both Formats 1 and 2 as depicted in FIG. 5. For both formats, anti-human Fc antibody was first loaded onto the sensor tips. For Format 1, Target A was loaded onto the anti-Fc-loaded sensor tip to a density that resulted in a wavelength shift of approximately 0.8 nanometer (nm). The antigen-loaded tips were then equilibrated off-line in assay buffer for one hour. The tips were then monitored on-line in assay buffer for approximately 60 seconds for baseline establishment. The tips were then exposed to approximately 100 nM of the bispecific antibody analog for approximately 300 seconds in order to bind to Target A. The bispecific antibody analog-loaded tips were transferred to assay buffer for approximately 300 seconds in order to monitor dissociation of the bispecific antibody analog from Target A. Binding kinetics were then analyzed using a 1:1 binding model. Measurements using Format 2 were carried out in the same manner except that Target B was loaded onto the anti-human Fc antibody-loaded tips. The results obtained with an exemplary bispecific antibody analog analyzed using Format 1 and Format 2 are provided in the lower left and lower right panels, respectively, of FIG. 5. The results demonstrate that the bivalent antibody analog binds both Target A and Target B with high affinity (Kd=630 μM and 6.3 nM, respectively).

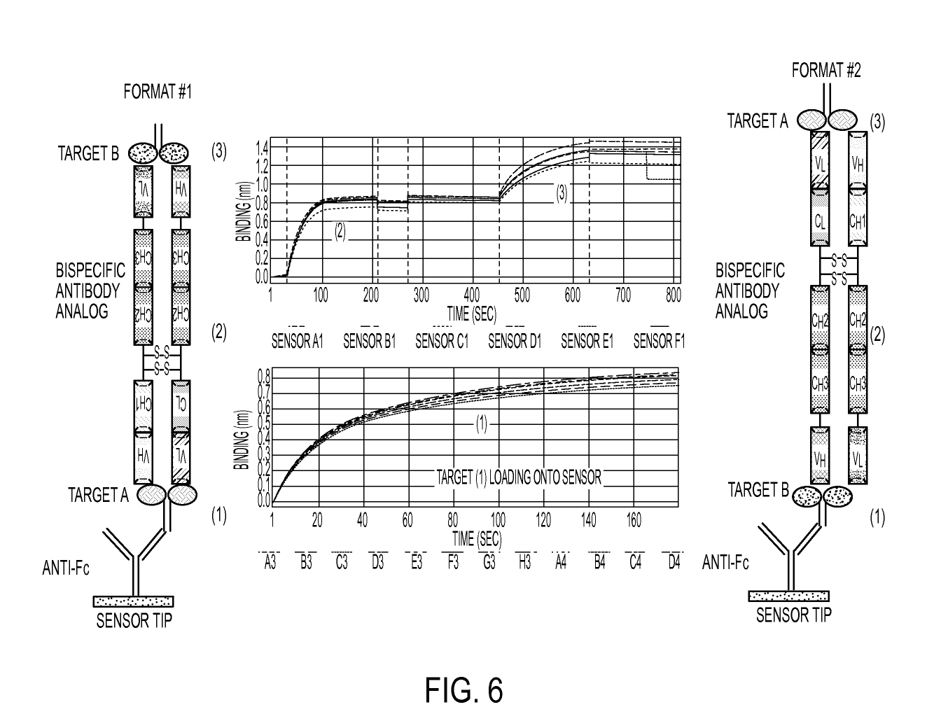

Simultaneous binding of five different exemplary antibody analogs to Targets A and B was also assessed using both Formats 1 and 2 as depicted in FIG. 6. For both formats, anti-human Fc antibody was loaded onto the sensor tips. For Format 1, Target A was loaded onto the anti-Fc-loaded sensor tip to a density that resulted in a wavelength shift of approximately 0.8 nanometer (nm). The antigen-loaded tips were then equilibrated off-line in assay buffer for one hour followed by application of a non-specific IgG saturate Fc binding sites that had not bound Target A. The tips were then monitored on-line in assay buffer for approximately 60 seconds for baseline establishment. The tips were then exposed to approximately 100 nM of the bispecific antibody analog for approximately 300 seconds in order to bind to Target A. The bispecific antibody analog-loaded tips were transferred to assay buffer for approximately 300 seconds in order to monitor dissociation of the bispecific antibody analog from Target A. The bispecific antibody analog-loaded tips were then exposed to a solution containing Target B for approximately 300 seconds to allow for binding of Target B to the bispecific antibody analog/Target A complex that was loaded onto the sensor tip. The Target B-loaded tip was then transferred into assay buffer for approximately 300 seconds in order to monitor dissociation of the Target B from the bispecific antibody analog-Target A complex. Measurements using Format 2 were carried out in the same manner except that Target B was loaded onto the anti-human Fc antibody-loaded tips.

The results obtained for multivalent analogs described above using Format 1 are provided in the upper middle and lower middle panels of FIG. 6. The results demonstrate that each bivalent antibody analog tested was able to bind both Target A and Target B simultaneously. Similar results were obtained when the experiments were performed according to Format 2 as represented in FIG. 6, right panel.

### Example 2

### Generation of Bispecific Antibody Analogs Comprising N-Terminal Antibody Binding Region (Fab) x C-Terminal Single-Chain Variable Region (scFv) that Binds to Target a Via the Fab and Target B Via the scFv

Generation of a Exemplary Constructs Encoding Polypeptide 1: N-Terminal VH-CH1-Fc Region-C-Terminal scFv Orientation

A mammalian expression vector suitable for expression in human embryonic kidney (HEK) cells and harboring the coding sequence for CH1, hinge, CH2, and CH3 regions of human IgG (e.g., as represented in FIG. 3C, right side) contains a unique Sfil restriction site and allows for in-frame recombination/cloning N-terminal to the CH1 region, as well as a NotI restriction site that allows for in-frame recombination/cloning C-terminal to the CH3 region. A sample of this vector was first digested with the Sfil enzyme. VH-encoding nucleic acid of an IgG that binds Target A (“VH-1”) was amplified using primers that contain 5′ and 3′ flanking regions, respectively, that are complimentary to the Sfil restriction-digested ends of the linearized vector. The amplified VH-encoding nucleic acid and the Sfil-digested vector were then ligated using T4 DNA ligase. E. coli cells were then transformed with the ligated vector via electroporation, allowed to recover for approximately one hour in SOC media at 37 degrees Celsius, and then plated on appropriate plates. Colonies were then picked from the plates, grown up, and plasmid DNA extracted and the sequence-confirmed to contain the VH-1, CH1, hinge, CH2, and CH3 regions as represented in FIG. 3C, right side. A sample of the recovered, sequence-verified vector containing the VH-1, CH1, hinge, CH2, and CH3 regions was then digested with the NotI enzyme in order to facilitate insertion of the scFv-encoding nucleic acids prepared as described immediately below.

Several different scFv-encoding nucleic acids were prepared, each of which contained nucleic acid encoding the same VH and VL regions which together form a binding site for Target B as well, as one of the following different linkers between the VH and VL regions:

Exemplary Linker 7.
(SEQ ID NO: 45)
Gly-Gly-Gly-Gly-Ser-Gly-Gly-Gly-Gly-Ser-Gly-Gly-
Gly-Gly-Ser-
Exemplary Linker 8.
(SEQ ID NO: 46)
Gly-Gly-Gly-Gly-Ser-Gly-Gly-Gly-Gly-Ser-Gly-Gly-
Gly-Gly-Ser-Gly-Gly-Gly-Gly-Ser
Exemplary Linker 9.
(SEQ ID NO: 47)
Gly-Gly-Gly-Gly-Ser-Gly-Gly-Gly-Gly-Ser-Gly-Gly-
Gly-Gly-Ser-Gly-Gly-Gly-Gly-Ser-Gly-Gly-Gly-Gly-
Ser
Exemplary Linker 10.
(SEQ ID NO: 48)
Gly-Gly-Gly-Gly-Ser-Gly-Gly-Gly-Gly-Ser-Gly-Gly-
Gly-Gly-Ser-Gly-Gly-Gly-Gly-Ser-Gly-Gly-Gly-Gly-
Ser-Gly-Gly-Gly-Gly-Ser.

Each of the scFv-encoding nucleic acids described above (including one of the linkers in between the VH and VL) also comprised one of the following different linkers N-terminal to the VH region of the scFv, which were incorporated into the scFv-encoding nucleic acid using 5′ primers as provided in the description of the preparation of the VH-2 and VL-2 encoding nucleic acid of Example:

Exemplary Linker 1.
(SEQ ID NO: 37)
Gly-Gly-Gly-Gly-Ser
Exemplary Linker 2.
(SEQ ID NO: 40)
Gly-Gly-Gly-Gly-Ser-Gly-Gly-Gly-Gly-Ser-Gly-Gly-
Gly-Gly
Exemplary Linker 3.
(SEQ ID NO: 41)
Gly-Gly-Gly-Gly-Ser-Gly-Gly-Gly-Gly-Ser-Gly-Gly-
Gly-Gly-Ser-Gly-Gly-Gly-Gly
Exemplary Linker 4.
(SEQ ID NO: 42)
Gly-Gly-Gly-Gly-Ser-Gly-Gly-Gly-Gly-Ser-Gly-Gly-
Gly-Gly-Ser-Gly-Gly-Gly-Gly-Ser-Gly-Gly-Gly-Gly
Exemplary Linker 5.
(SEQ ID NO: 43)
Gly-Gly-Gly-Gly-Ser-Gly-Gly-Gly-Gly-Ser-Gly-Gly-
Gly-Gly-Ser-Gly-Gly-Gly-Gly-Ser-Gly-Gly-Gly-Gly-
Ser-Gly-Gly-Gly-Gly
Exemplary Linker 6.
(SEQ ID NO: 44)
Gly-Gly-Gly-Gly-Ser-Gly-Gly-Gly-Gly-Ser-Gly-Gly-
Gly-Gly-Ser-Gly-Gly-Gly-Gly-Ser-Gly-Gly-Gly-Gly-
Ser-Gly-Gly-Gly-Gly-Ser-Gly-Gly-Gly-Gly.

Each 5′ primer also contained nucleic acid that was complimentary to the NotI restriction-digested ends of the linearized vector at the 5′ end of the primer (i.e., upstream of the linker-encoding sequence). A 3′-primer was also prepared that contained, at its 3′ end, nucleic acid that encoded a six-histidine tag (SEQ ID NO: 38) (“His tag”), followed by a downstream sequence that was complimentary to the NotI restriction-digested ends of the linearized vector. The 3′ primer also contained appropriate coding sequence upstream of the His tag that was complimentary to the coding sequence for the VL region from the IgG that binds Target B (“VL-2”).

The scFvs described above were then amplified using, in separate reactions, each 5′ primer (each one containing one of the linker-encoding sequences as described above) and the 3′ primer. The amplified scFv (comprising nucleic acid encoding a linker, VH-2, linker VL-2)-encoding nucleic acid and the Sfil-digested vector were then ligated using T4 DNA ligase. E. coli cells were then transformed with the ligated vector via electroporation, allowed to recover for approximately one hour in SOC media at 37 degrees Celsius, and then plated on appropriate plates. Colonies were then picked from the plates, grown up, and plasmid DNA extracted and the sequence-confirmed to contain the VH-1, CH1, hinge, CH2, CH3 and scFv (comprising nucleic acid encoding a linker, VH-2, linker VL-2) regions as represented in FIG. 3C, right side.

### Generation of a Exemplary Constructs Encoding Polypeptide 2: N-Terminal VL-CL-Fc Region Orientation

A vector harboring the coding sequence for CL, hinge, CH2, and CH3 regions of human IgG (e.g., as represented in FIG. 3C, left side) contains a unique Sfil restriction site and allows for in-frame recombination/cloning N-terminal to the CL region, as well as a NotI restriction site that allows for in-frame recombination/cloning C-terminal to the CH3 region. A sample of this vector was first digested with the Sfil enzyme. VL-encoding nucleic acid of an IgG that binds Target A (“VL-1”) was amplified using primers that contain 5′ and 3′ flanking regions, respectively, that are complimentary to the Sfil restriction-digested ends of the linearized vector. The amplified VH-encoding nucleic acid and the Sfil-digested vector were then ligated using T4 DNA ligase. E. coli cells were then transformed with the ligated vector via electroporation, allowed to recover for approximately one hour in SOC media at 37 degrees Celsius, and then plated on appropriate plates. Colonies were then picked from the plates, grown up, and plasmid DNA extracted and the sequence-confirmed to contain the VL-1, CL, hinge, CH2, and CH3 regions as represented in FIG. 3C, left side.

### Expression of the Bispecific Antibody Analogs

Human embryonic kidney (HEK) cells were transfected with vectors encoding polypeptide 1 and polypeptide 2, prepared as described above, and the bispecific antibody analogs expressed. A series of vectors encoding polypeptides 1 and 2 were also prepared as described above in which the N-terminal Fab portion of the bispecific analogs expressed thereby bind Target B, and the C-terminal scFv portions bind Target A.

### Purification of Bispecific Antibody Analogs

A three-step purification scheme, provided below, was employed in order to purify each of the antibody analogs described above.

### Protein A Purification:

Protein A resin (MabSelect SuRe (GE Healthcare) was employed as the first purification step for each bispecific antibody analog. The resin was first equilibrated in wash buffer (phosphate buffered saline, pH 7.4). Sample containing the bispecific antibody analog was applied to the column. The column was washed several times with wash buffer after addition of the sample containing the bispecific antibody analog to the column. The bispecific antibody analog was then eluted from the column by the addition of elution buffer (200 mM Acetic Acid, pH 2.0). Once eluted, the sample was neutralized with 2 M HEPES, pH 8.0.

### Ceramic Hydroxyapatite (CHT) Purification:

Bio-Rad Macro-Prep Ceramic Hydroxyapatite TYPE 140 μm resin was employed as the second purification step for each bispecific antibody analog. The resin was first equilibrated with 10 column volumes of wash buffer (20 mM NaH₂PO₄, pH7.0). Sample containing the bispecific antibody analog was applied to the column. After addition of the sample to the column, the column was washed with two column volumes of wash buffer. A linear gradient from 0-100% over 20 column volumes was used for to elute the sample, collecting 4 mL fractions.

### HisTag Purification:

HisTag resin (Ni Sepharose 6 Fast Flow, (GE Healthcare) was employed as the third purification step for each bispecific antibody analog. The resin was first equilibrated in wash buffer (20 mM NaH₂PO₄, 500 mM NaCl, 20 mM Imidazole, pH 7.4). Sample containing the bispecific antibody analog was applied to the column. After addition of the sample to the column, the column was washed several times with wash buffer. The bispecific antibody analog was then eluted from the column by the addition of elution buffer (20 mM NaH₂PO₄, 500 mM NaCl, 500 mM Imidazole, pH 7.4).

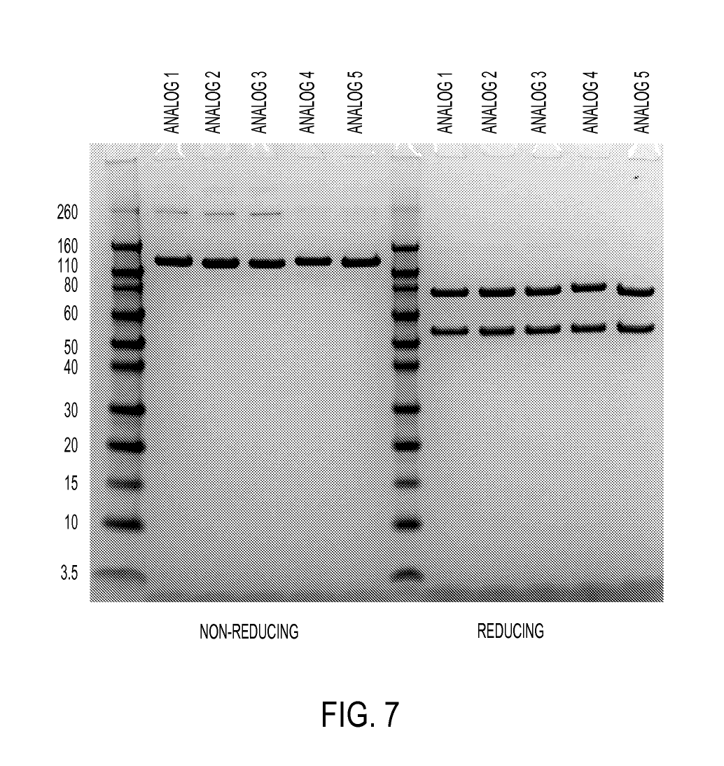

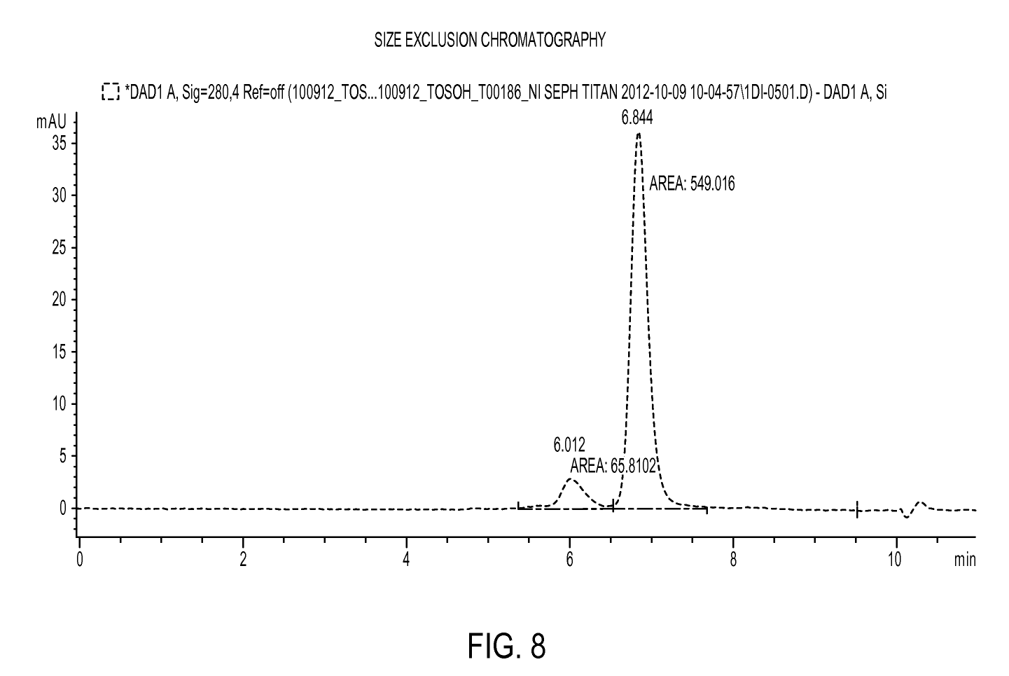

Expression of each of the antibody analogs was confirmed by both reducing and non-reducing polyacrylamide gel electrophoresis (PAGE) (see, e.g., FIG. 7) and size exclusion chromatography (SEC) in accordance with methodologies known in the art. Briefly, with regard to SEC, an Agilent 1100 HPLC was employed to monitor the column chromatography (TSKgel Super SW3000 column). The column was equilibrated with wash buffer (200 mM Sodium Phosphate, 250 mM Sodium Chloride pH 6.8) prior to use. Approximately 2-5 μg of protein sample was injected onto column and flow rate adjusted to 0.400 mL/min. Protein migration was monitored at wavelength 280 nm. Total assay time was approximately 11 min. The SEC profile of an exemplary bispecific antibody analog with the N-terminal antibody fragment (Fab) x C-terminal variable fragment (Fᵥ) is depicted in FIG. 8, in which the major peak, corresponding to the desired multivalent antibody analog, is evident.

### Binding and Affinity Measurements

This material was collected and used to carry out binding and affinity measurements using a Forte-Bio Octet Red 384 instrument generally in accordance with the manufacturer's instructions.

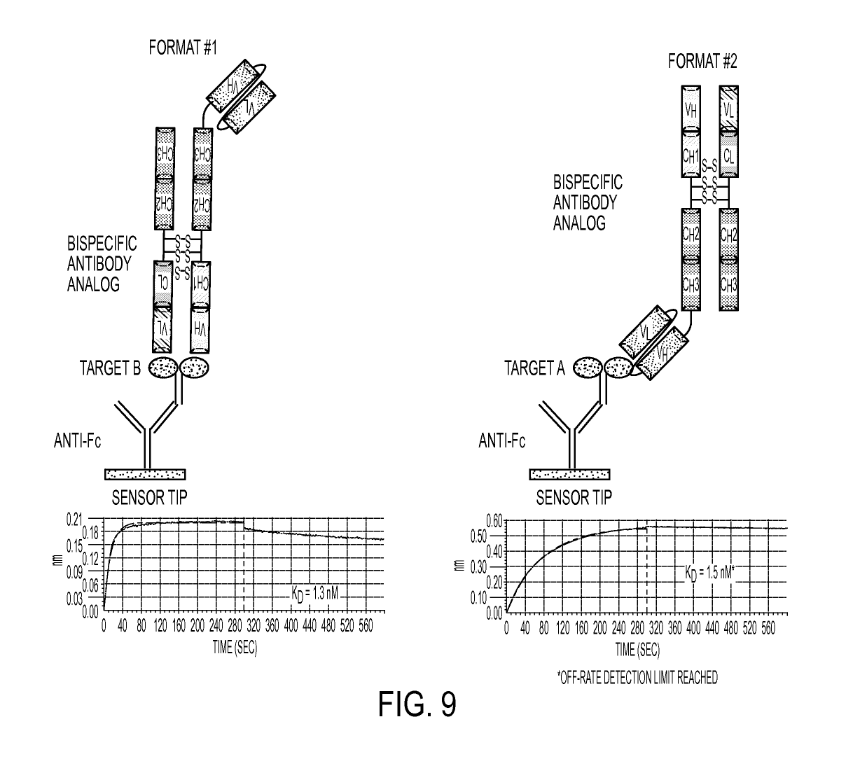

Monovalent binding and affinity measurements were first made using both Formats 1 and 2 as depicted in FIG. 9. For both formats, anti-human Fc antibody was first loaded onto the sensor tips. For Format 1, Target A was loaded onto the anti-Fc-loaded sensor tip to a density that resulted in a wavelength shift of approximately 0.8 nanometer (nm). The antigen-loaded tips were then equilibrated off-line in assay buffer for one hour. The tips were then monitored on-line in assay buffer for approximately 60 seconds for baseline establishment. The tips were then exposed to approximately 100 nM of the bispecific antibody analog for approximately 300 seconds in order to bind to Target A. The bispecific antibody analog-loaded tips were transferred to assay buffer for approximately 300 seconds in order to monitor dissociation of the bispecific antibody analog from Target A. Binding kinetics were then analyzed using a 1:1 binding model. Measurements using Format 2 were carried out in the same manner except that Target B was loaded onto the anti-human Fc antibody-loaded tips. The results obtained with an exemplary bispecific antibody analog analyzed using Format 1 and Format 2 are provided in the lower left and lower right panels, respectively, of FIG. 9. The results demonstrate that the bivalent antibody analog binds both Target A and Target B with high affinity (Kd=1.3 nM and 1.5 nM, respectively).

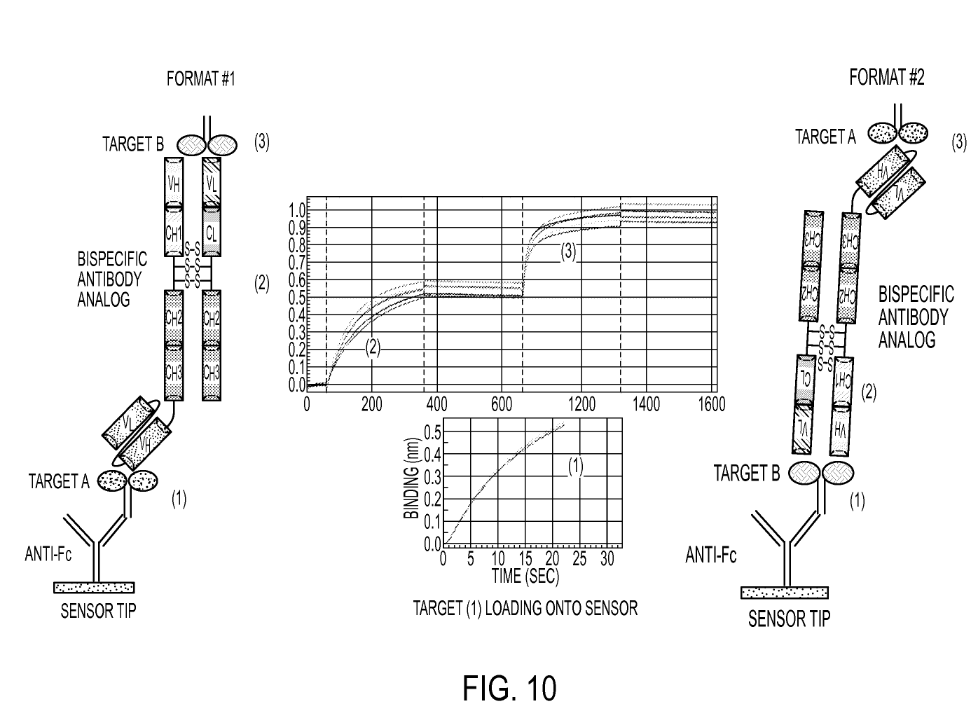

Simultaneous binding of five different exemplary antibody analogs to Targets A and B was also assessed using both Formats 1 and 2 as depicted in FIG. 10. For both formats, anti-human Fc antibody was loaded onto the sensor tips. For Format 1, Target A was loaded onto the anti-Fc-loaded sensor tip to a density that resulted in a wavelength shift of approximately 0.8 nanometer (nm). The antigen-loaded tips were then equilibrated off-line in assay buffer for one hour followed by application of a non-specific IgG saturate F_{c} binding sites that had not bound Target A. The tips were then monitored on-line in assay buffer for approximately 60 seconds for baseline establishment. The tips were then exposed to approximately 100 nM of the bispecific antibody analog for approximately 300 seconds in order to bind to Target A. The bispecific antibody analog-loaded tips were transferred to assay buffer for approximately 300 seconds in order to monitor dissociation of the bispecific antibody analog from Target A. The bispecific antibody analog-loaded tips were then exposed to a solution containing Target B for approximately 300 seconds to allow for binding of Target B to the bispecific antibody analog/Target A complex that was loaded onto the sensor tip. The Target B-loaded tip was then transferred into assay buffer for approximately 300 seconds in order to monitor dissociation of the Target B from the bispecific antibody analog-Target A complex. Measurements using Format 2 were carried out in the same manner except that Target B was loaded onto the anti-human Fc antibody-loaded tips.

The results obtained for multivalent analogs described above using Format 1 ARE provided in the upper middle and lower middle panels of FIG. 10. The results demonstrate that each bivalent antibody analog tested was able to bind both Target A and Target B simultaneously. Similar results were obtained when the experiments were performed according to Format 2 as represented in FIG. 10, right panel.

Whereas particular Embodiments of the invention have been described above for purposes of illustration, it will be appreciated by those skilled in the art that numerous variations of the details may be made without departing from the invention as described in the appended claims.

1. A multivalent antibody analog comprising a first polypeptide and a second polypeptide, wherein:
a) the first polypeptide comprises a first heavy chain comprising a variable heavy region, a CH2 domain or a variant thereof, and a CH3 domain or a variant thereof; wherein the C-terminus of the CH3 domain or variant thereof is covalently attached to a first variable light domain (V_{L}); and
b) the second polypeptide comprises a first light chain covalently attached to the N-terminus of an Fc region of a heavy chain, wherein the Fc region comprises a CH2 domain or a variant thereof and a CH3 domain or a variant thereof, and wherein the CH3 domain or variant thereof is covalently attached to a first variable heavy domain (V_{H});
wherein said first heavy chain and said first light chain form a first antigen binding site and said first V_{L} and said first V_{H} form a second antigen binding site.

2. The multivalent antibody analog according to claim 1, wherein the first polypeptide and the second polypeptide each further comprises a hinge region, and wherein said hinge regions each contain at least one thiol group that is capable of participating in an intermolecular disulfide bond such that the first and the second polypeptide are covalently linked as a result of formation of the disulfide bond.

3. (canceled)

4. The multivalent antibody analog according to claim 1 or claim 2, wherein the first VL is covalently attached to the CH3 domain, or variant thereof, of the first heavy chain via a linker moiety.

5. The multivalent antibody analog according to claim 1 or claim 2, wherein the first VH is covalently attached to the CH3 domain, or variant thereof, of the Fc region via a linker moiety.

6. The multivalent antibody analog according to claim 1 or claim 2, wherein the antibody analog further comprises a third antigen binding site.

7. The multivalent antibody analog according to claim 6, wherein the third antigen binding site is covalently attached via a linker moiety to either:
the first VL; or
the first VH.

8. The multivalent antibody analog according to claim 6, wherein the third antigen binding site comprises a single chain variable region (scFv), wherein said scFv comprises a second VL that is covalently attached to a second VH.

9. The multivalent antibody analog according to claim 8, wherein the second VL is covalently attached to the second VH via a linker moiety.

10. The multivalent antibody analog according to claim 9, wherein:
the second VL is attached to the first VL via a linker moiety;
the second VH is attached to the first VH via a linker moiety;
the second VL is attached to the first VH via a linker moiety; or
the second VH is attached to the first VL via a linker moiety.

11. The multivalent antibody analog according to claim 1, wherein the antibody analog further comprises a fourth antigen binding site.

12. The multivalent antibody analog according to claim 11, wherein the fourth antigen binding site is covalently attached either:
the first VL via a linker moiety; or
the first VH via a linker moiety.

13. The multivalent antibody analog according to claim 11, wherein the fourth antigen binding site comprises a second single chain variable region (scFv), wherein said second scFv comprises a third VL that is covalently attached to a third VH.

14. The multivalent antibody analog according to claim 13, wherein the third VL is covalently attached to the third VH via a linker moiety.

15. The multivalent antibody analog according to claim 14, wherein:
the third VL is attached to the first VL via a linker moiety;
the third VH is attached to the first VH via a linker moiety;
the third VL is attached to the first VH via a linker moiety; or
the third VH is attached to the first VL via a linker moiety.

16. The multivalent antibody analog according to claim 15, wherein one or more of the linker moieties independently comprises a peptide from 1 to 75 amino acids in length, inclusive.

17.-18. (canceled)

19. The multivalent antibody analog according to claim 15, wherein the one or more of the linker moieties independently comprises one or more amino acids selected from the group consisting of serine, glycine, alanine, proline, asparagine, glutamine, glutamate, aspartate, and lysine.

20.-24. (canceled)

25. The multivalent antibody analog according to claim 15, wherein one or more of the linker moieties independently comprises a combination of Gly and Ala.

26. The multivalent antibody analog according to claim 15, wherein one or more of the linker moieties independently comprises a combination of Gly and Ser.

27. The multivalent antibody analog according to claim 15, wherein one or more of the linker moieties independently comprises a combination of:
Gly and Glu; or
Gly and Asp.

28. The multivalent antibody analog according to claim 15, wherein one or more of the linker moieties independently comprises a combination of Gly and Lys.

29. The multivalent antibody analog according to claim 15, wherein one or more of the linker moieties independently comprises a sequence selected from group consisting of: [Gly-Ser]ₙ (SEQ ID NO: 6); [Gly-Gly-Ser]ₙ (SEQ ID NO: 7); [Gly-Gly-Gly-Ser]ₙ (SEQ ID NO: 8); [Gly-Gly-Gly-Gly-Ser]ₙ (SEQ ID NO: 9); [Gly-Gly-Gly-Gly-Ser-Gly-Gly-Gly-Gly]ₙ (SEQ ID NO: 10); [Gly-Gly-Gly-Gly-Ser-Gly-Gly-Gly-Gly-Ser-Gly-Gly-Gly-Gly]ₙ (SEQ ID NO: 11); [Gly-Gly-Gly-Gly-Ser Gly-Gly-Gly-Gly-Ser-Gly-Gly-Gly-Gly-Ser-Gly-Gly-Gly-Gly]ₙ (SEQ ID NO: 12); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 13); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 14); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 15); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

30. The multivalent antibody analog according to claim 15, wherein one or more of the linker moieties independently comprises a sequence selected from the group consisting of: [Gly-Glu]ₙ (SEQ ID NO: 16); [Gly-Gly-Glu]ₙ (SEQ ID NO: 17); [Gly-Gly-Gly-Glu]ₙ (SEQ ID NO: 18); [Gly-Gly-Gly-Gly-Glu]ₙ (SEQ ID NO: 19); [Gly-Asp]ₙ (SEQ ID NO: 20); [Gly-Gly-Asp]ₙ (SEQ ID NO: 21); [Gly-Gly-Gly-Asp]ₙ (SEQ ID NO: 22); [Gly-Gly-Gly-Gly-Asp]ₙ (SEQ ID NO: 23); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

31. The multivalent antibody analog according to claim 1 or claim 2, wherein the CH2 domain variant and the CH3 domain variant each independently comprises at least one different amino acid substitution such that a heterodimeric domain pair is generated such that heterodimerization of said variants is favored over homodimerization.

32.-33. (canceled)

34. A multivalent antibody analog comprising a first polypeptide and a second polypeptide, wherein:
a) the first polypeptide comprises a first heavy chain comprising a variable heavy region, a CH2 domain or a variant thereof, and a CH3 domain or a variant thereof;
b) the second polypeptide comprises a first light chain covalently attached to the N-terminus of an Fc region of a heavy chain, wherein the Fc region comprises a CH2 domain or a variant thereof and a CH3 domain or a variant thereof; and
c) either the first polypeptide or the second polypeptide further comprises a single chain variable region (scFv) comprising a first VL that is covalently attached to a first VH, wherein said scFv is covalently attached to the CH3 domain or variant thereof of said first polypeptide or said second polypeptide;
wherein said first heavy chain and said first light chain form a first antigen binding site and the first VL and the first VH form a second antigen binding site.

35. A multivalent antibody analog comprising a first polypeptide and a second polypeptide, wherein:
a) the first polypeptide comprises a first heavy chain comprising a variable heavy region, a CH2 domain or a variant thereof, and a CH3 domain or a variant thereof;
b) the second polypeptide comprises a first light chain covalently attached to the N-terminus of an Fc region of a heavy chain, wherein the Fc region comprises a CH2 domain or a variant thereof and a CH3 domain or a variant thereof; and
c) the first polypeptide further comprises a single chain variable region (scFv) comprising a first VL that is covalently attached to a first VH; and the second polypeptide further comprises a single chain variable region (scFv) comprising a second VL that is covalently attached to a second VH; wherein one scFv is covalently attached to the CH3 domain of variant thereof of said first polypeptide and the other scFv is covalently attached to the CH3 domain or variant thereof of said second polypeptide;
wherein said first heavy chain and said first light chain form a first antigen binding site, the first VL and the first VH form a second antigen binding site, and the second VL and the second VH form a third antigen binding site.

36. The multivalent antibody analog according to claim 34 or claim 35, wherein the first polypeptide and the second polypeptide each further comprises a hinge region, and wherein said hinge regions each contain at least one thiol group that is capable of participating in an intermolecular disulfide bond such that the first and the second polypeptides are covalently linked as a result of formation of the disulfide bond.

37. (canceled)

38. The multivalent antibody analog according to claim 34 or claim 35, wherein:
the first VL is attached to the CH3 domain, or variant thereof, of the first heavy chain via a linker moiety;
the first VL is attached to the CH3 domain, or variant thereof, of the Fc region via a linker moiety;
the first VH is attached to the CH3 domain, or variant thereof, of the first heavy chain via a linker moiety; or
the first VH is attached to the CH3 domain, or variant thereof, of the Fc region via a linker moiety.

39. The multivalent antibody analog according to claim 34 or claim 35, wherein:
the second VL is attached to the CH3 domain, or variant thereof, of the first heavy chain via a linker moiety;
the second VL is attached to the CH3 domain, or variant thereof, of the Fc region via a linker moiety;
the second VH is attached to the CH3 domain, or variant thereof, of the first heavy chain via a linker moiety; or
the second VH is attached to the CH3 domain, or variant thereof, of the Fc region via a linker moiety.

40. The multivalent antibody analog according to claim 38, wherein one or more of the linker moieties independently comprises a peptide from 1 to 75 amino acids in length, inclusive.

41.-42. (canceled)

43. The multivalent antibody analog according to claim 40, wherein the one or more of the linker moieties independently comprises one or more amino acids selected from the group consisting of serine, glycine, alanine, proline, asparagine, glutamine, glutamate, aspartate, and lysine.

44.-48. (canceled)

49. The multivalent antibody analog according to claim 40, wherein one or more of the linker moieties independently comprises a combination of Gly and Ala.

50. The multivalent antibody analog according to claim 40, wherein one or more of the linker moieties independently comprises a combination of Gly and Ser.

51. The multivalent antibody analog according to claim 40, wherein one or more of the linker moieties independently comprises a combination of:
Gly and Glu; or
Gly and Asp.

52. (canceled)

53. The multivalent antibody analog according to claim 40, wherein one or more of the linker moieties independently comprises a sequence selected from group consisting of: [Gly-Ser]ₙ (SEQ ID NO: 6); [Gly-Gly-Ser]ₙ (SEQ ID NO: 7); [Gly-Gly-Gly-Ser]ₙ (SEQ ID NO: 8); [Gly-Gly-Gly-Gly-Ser]ₙ (SEQ ID NO: 9); [Gly-Gly-Gly-Gly-Ser-Gly-Gly-Gly-Gly]ₙ (SEQ ID NO: 10); [Gly-Gly-Gly-Gly-Ser-Gly-Gly-Gly-Gly-Ser-Gly-Gly-Gly-Gly]ₙ (SEQ ID NO: 11); [Gly-Gly-Gly-Gly-Ser Gly-Gly-Gly-Gly-Ser-Gly-Gly-Gly-Gly-Ser-Gly-Gly-Gly-Gly]ₙ (SEQ ID NO: 12); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 13); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 14); [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ (SEQ ID NO: 15); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

54. The multivalent antibody analog according to claim 40, wherein one or more of the linker moieties independently comprises a sequence selected from the group consisting of: [Gly-Glu]ₙ (SEQ ID NO: 16); [Gly-Gly-Glu]ₙ (SEQ ID NO: 17); [Gly-Gly-Gly-Glu]ₙ (SEQ ID NO: 18); [Gly-Gly-Gly-Gly-Glu]ₙ (SEQ ID NO: 19); [Gly-Asp]n (SEQ ID NO: 20); [Gly-Gly-Asp]ₙ (SEQ ID NO: 21); [Gly-Gly-Gly-Asp]ₙ (SEQ ID NO: 22); [Gly-Gly-Gly-Gly-Asp]ₙ (SEQ ID NO: 23); and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

55. The multivalent antibody analog according to claim 34 or claim 35, wherein the CH2 domain variant and the CH3 domain variant each independently comprises at least one different amino acid substitution such that a heterodimeric domain pair is generated such that heterodimerization of said variants is favored over homodimerization.

56.-57. (canceled)

58. The multivalent antibody analog according to any one of claims 1, 2, 34 and 35, wherein at least one antigen binding site comprises at least one humanized variable heavy domain or at least one humanized variable light domain.

59. The multivalent antibody analog according to any one of claims 1, 2, 34 and 35, wherein at least one antigen binding site comprises at least one complimentary determining region CDR that is derived from a non-human antibody or antibody fragment.

60.-64. (canceled)

65. The multivalent antibody analog according to any one of claims 1, 2, 34 and 35, wherein at least one antigen binding site binds an epitope from a tumor associated antigen, a hormone receptor, a cytokine receptor, chemokine receptor, a growth factor receptor, an immune activating receptor, a hormone, a cytokine, a chemokine, a growth factor, a G protein-coupled receptor, or a transmembrane receptor.

66. The multivalent antibody analog according to any one of claims 1, 2, 34 and 35, wherein at least one antigen binding site binds a target associated with an autoimmune disorder, an inflammatory disorder, an oncological disorder, neuromuscular disorder, a neurodegenerative disorder, a metabolic disorder, or an infectious disease.

67. The multivalent antibody analog according to any one of claims 1, 2, 34 and 35, wherein the antibody analog binds at least two different targets.

68.-69. (canceled)

70. The multivalent antibody analog according to any one of claims 1, 2, 34 and 35, wherein the antibody analog binds at least one target monovalently.

71.-76. (canceled)

77. The multivalent antibody analog according to any one of claims 1, 2, 34 and 35, wherein:
a) the first polypeptide further comprises a CH1 domain or a variant thereof covalently attached to the CH2 domain or a variant thereof;
b) the first light chain of the second polypeptide comprises either a Vkappa domain or a Vlambda domain covalently attached to C-terminus of the VL domain and to the N-terminus of an Fc region of the heavy chain.

78. A method of treating an autoimmune disorder, an inflammatory disorder, an oncological disorder, neuromuscular disorder, a neurodegenerative disorder, a metabolic disorder, or an infectious disease, the method comprising providing or administering a therapeutically effective amount of a multivalent antibody analog according to any one of claims 1, 2, 34 and 35.

79. (canceled)

80. The multivalent antibody analog according to claim 2, wherein the antibody analog further comprises a fourth antigen binding site.

81. The multivalent antibody analog according to claim 39, wherein one or more of the linker moieties independently comprises a peptide from 1 to 75 amino acids in length, inclusive.

82. The multivalent antibody analog according to claim 81, wherein the one or more of the linker moieties independently comprises one or more amino acids selected from the group consisting of serine, glycine, alanine, proline, asparagine, glutamine, glutamate, aspartate, and lysine.

83. The multivalent antibody analog according to claim 81, wherein one or more of the linker moieties independently comprises a combination of Gly and Ala.

84. The multivalent antibody analog according to claim 81, wherein one or more of the linker moieties independently comprises a combination of Gly and Ser.

85. The multivalent antibody analog according to claim 81, wherein one or more of the linker moieties independently comprises a combination of:
Gly and Glu; or
Gly and Asp.

86. The multivalent antibody analog according to claim 81, wherein one or more of the linker moieties independently comprises a sequence selected from group consisting of: [Gly-Ser]ₙ; [Gly-Gly-Ser]ₙ; [Gly-Gly-Gly-Ser]ₙ; [Gly-Gly-Gly-Gly-Ser]ₙ; [Gly-Gly-Gly-Gly-Ser-Gly-Gly-Gly-Gly]ₙ; [Gly-Gly-Gly-Gly-Ser-Gly-Gly-Gly-Gly-Ser-Gly-Gly-Gly-Gly]ₙ; [Gly-Gly-Gly-Gly-Ser Gly-Gly-Gly-Gly-Ser-Gly-Gly-Gly-Gly-Ser-Gly-Gly-Gly-Gly]ₙ; [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ; [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ; [Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly]ₙ; and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.

87. The multivalent antibody analog according to claim 81, wherein one or more of the linker moieties independently comprises a sequence selected from the group consisting of: [Gly-Glu]ₙ; [Gly-Gly-Glu]ₙ; [Gly-Gly-Gly-Glu]ₙ; [Gly-Gly-Gly-Gly-Glu]ₙ; [Gly-Asp]ₙ; [Gly-Gly-Asp]ₙ; [Gly-Gly-Gly-Asp]ₙ; [Gly-Gly-Gly-Gly-Asp]ₙ; and combinations thereof; where n is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, and 75.


SEQUENCE LISTING (48 sequences)

SEQ ID NO: 1 - protein, 4 aa, Artificial Sequence
  Description of Artificial Sequence Synthetic peptide
GGGG

SEQ ID NO: 2 - protein, 5 aa, Artificial Sequence
  Description of Artificial Sequence Synthetic peptide
GGGGG

SEQ ID NO: 3 - protein, 8 aa, Artificial Sequence
  Description of Artificial Sequence Synthetic peptide
GGGKGGGG

SEQ ID NO: 4 - protein, 8 aa, Artificial Sequence
  Description of Artificial Sequence Synthetic peptide
GGGNGSGG

SEQ ID NO: 5 - protein, 8 aa, Artificial Sequence
  Description of Artificial Sequence Synthetic peptide
GGGCGGGG

SEQ ID NO: 6 - protein, 150 aa, Artificial Sequence
  Description of Artificial Sequence Synthetic polypeptide
GSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGSGS

SEQ ID NO: 7 - protein, 225 aa, Artificial Sequence
  Description of Artificial Sequence Synthetic polypeptide
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 8 - protein, 300 aa, Artificial Sequence
  Description of Artificial Sequence Synthetic polypeptide
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 9 - protein, 375 aa, Artificial Sequence
  Description of Artificial Sequence Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 10 - protein, 675 aa, Artificial Sequence
  Description of Artificial Sequence Synthetic polypeptide
GGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGGGGGGSGGGG

SEQ ID NO: 11 - protein, 1050 aa, Artificial Sequence
  Description of Artificial Sequence Synthetic polypeptide
GGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGGGGGGSGGGGSGGGG

SEQ ID NO: 12 - protein, 1425 aa, Artificial Sequence
  Description of Artificial Sequence Synthetic polypeptide
GGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGGGGGGSGGGGSGGGGSGGGG

SEQ ID NO: 13 - protein, 1800 aa, Artificial Sequence
  Description of Artificial Sequence Synthetic polypeptide
GGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGGGGGGSGGGGSGGGGSGGGGSGGGG

SEQ ID NO: 14 - protein, 2175 aa, Artificial Sequence
  Description of Artificial Sequence Synthetic polypeptide
GGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGGGGGGSGGGGSGGGGSGGGGSGGGGSGGGG

SEQ ID NO: 15 - protein, 2550 aa, Artificial Sequence
  Description of Artificial Sequence Synthetic polypeptide
GGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGGGGGGSGGGGSGGGGSGGGGSGGGGSGGGGSGGGG

SEQ ID NO: 16 - protein, 150 aa, Artificial Sequence
  Description of Artificial Sequence Synthetic polypeptide
GEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGEGE

SEQ ID NO: 17 - protein, 225 aa, Artificial Sequence
  Description of Artificial Sequence Synthetic polypeptide
GGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGEGGE

SEQ ID NO: 18 - protein, 300 aa, Artificial Sequence
  Description of Artificial Sequence Synthetic polypeptide
GGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGEGGGE

SEQ ID NO: 19 - protein, 375 aa, Artificial Sequence
  Description of Artificial Sequence Synthetic polypeptide
GGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGEGGGGE

SEQ ID NO: 20 - protein, 150 aa, Artificial Sequence
  Description of Artificial Sequence Synthetic polypeptide
GDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGDGD

SEQ ID NO: 21 - protein, 225 aa, Artificial Sequence
  Description of Artificial Sequence Synthetic polypeptide
GGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGDGGD

SEQ ID NO: 22 - protein, 300 aa, Artificial Sequence
  Description of Artificial Sequence Synthetic polypeptide
GGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGDGGGD

SEQ ID NO: 23 - protein, 375 aa, Artificial Sequence
  Description of Artificial Sequence Synthetic polypeptide
GGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGDGGGGD

SEQ ID NO: 24 - protein, 5 aa, Artificial Sequence
  Description of Artificial Sequence Synthetic peptide
GPNGG

SEQ ID NO: 25 - protein, 150 aa, Artificial Sequence
  Description of Artificial Sequence Synthetic polypeptide
GKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGKGK

SEQ ID NO: 26 - protein, 225 aa, Artificial Sequence
  Description of Artificial Sequence Synthetic polypeptide
GGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGKGGK

SEQ ID NO: 27 - protein, 300 aa, Artificial Sequence
  Description of Artificial Sequence Synthetic polypeptide
GGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGKGGGK

SEQ ID NO: 28 - protein, 375 aa, Artificial Sequence
  Description of Artificial Sequence Synthetic polypeptide
GGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGKGGGGK

SEQ ID NO: 29 - protein, 150 aa, Artificial Sequence
  Description of Artificial Sequence Synthetic polypeptide
GRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGR

SEQ ID NO: 30 - protein, 225 aa, Artificial Sequence
  Description of Artificial Sequence Synthetic polypeptide
GGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGRGGR

SEQ ID NO: 31 - protein, 300 aa, Artificial Sequence
  Description of Artificial Sequence Synthetic polypeptide
GGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGRGGGR

SEQ ID NO: 32 - protein, 375 aa, Artificial Sequence
  Description of Artificial Sequence Synthetic polypeptide
GGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGRGGGGR

SEQ ID NO: 33 - protein, 375 aa, Artificial Sequence
  Description of Artificial Sequence Synthetic polypeptide
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 34 - protein, 15 aa, Artificial Sequence
  Description of Artificial Sequence Synthetic peptide
EAAAKEAAAKEAAAK

SEQ ID NO: 35 - protein, 20 aa, Artificial Sequence
  Description of Artificial Sequence Synthetic peptide
EAAAKEAAAKEAAAKEAAAK

SEQ ID NO: 36 - protein, 25 aa, Artificial Sequence
  Description of Artificial Sequence Synthetic peptide
EAAAKEAAAKEAAAKEAAAKEAAAK

SEQ ID NO: 37 - protein, 5 aa, Artificial Sequence
  Description of Artificial Sequence Synthetic peptide
GGGGS

SEQ ID NO: 38 - protein, 6 aa, Artificial Sequence
  Description of Artificial Sequence Synthetic peptide
HHHHHH

SEQ ID NO: 39 - protein, 10 aa, Artificial Sequence
  Description of Artificial Sequence Synthetic peptide
HHHHHHHHHH

SEQ ID NO: 40 - protein, 14 aa, Artificial Sequence
  Description of Artificial Sequence Synthetic peptide
GGGGSGGGGSGGGG

SEQ ID NO: 41 - protein, 19 aa, Artificial Sequence
  Description of Artificial Sequence Synthetic peptide
GGGGSGGGGSGGGGSGGGG

SEQ ID NO: 42 - protein, 24 aa, Artificial Sequence
  Description of Artificial Sequence Synthetic peptide
GGGGSGGGGSGGGGSGGGGSGGGG

SEQ ID NO: 43 - protein, 29 aa, Artificial Sequence
  Description of Artificial Sequence Synthetic peptide
GGGGSGGGGSGGGGSGGGGSGGGGSGGGG

SEQ ID NO: 44 - protein, 34 aa, Artificial Sequence
  Description of Artificial Sequence Synthetic polypeptide
GGGGSGGGGSGGGGSGGGGSGGGGSGGGGSGGGG

SEQ ID NO: 45 - protein, 15 aa, Artificial Sequence
  Description of Artificial Sequence Synthetic peptide
GGGGSGGGGSGGGGS

SEQ ID NO: 46 - protein, 20 aa, Artificial Sequence
  Description of Artificial Sequence Synthetic peptide
GGGGSGGGGSGGGGSGGGGS

SEQ ID NO: 47 - protein, 25 aa, Artificial Sequence
  Description of Artificial Sequence Synthetic peptide
GGGGSGGGGSGGGGSGGGGSGGGGS

SEQ ID NO: 48 - protein, 30 aa, Artificial Sequence
  Description of Artificial Sequence Synthetic polypeptide
GGGGSGGGGSGGGGSGGGGSGGGGSGGGGS